SEQUENCE LISTING (118 sequences)

SEQ ID NO: 1 - protein, 194 aa, Homo sapiens
MTENSTSAPAAKPKRAKASKKSTDHPKYSDMIVAAIQAEKNRAGSSRQSIQKYIKSHYKVGENADSQIKLSIKRLVTTGVLKQTKGVGASGSFRLAKSDEPKKSVAFKKTKKEIKKVATPKKASKPKKAASKAPTKKPKATPVKKAKKKLAATPKKAKKPKTVKAKPVKASKPKKAKPVKPKAKSSAKRAGKKK

SEQ ID NO: 2 - protein, 194 aa, synthetic construct; identical to SEQ ID NO: 1
  REGION 1..194: note = Human H1.0 Protein Dimethylated at K180
  MOD_RES 180: note = dimethyllysine

SEQ ID NO: 3 - protein, 18 aa, synthetic construct
  REGION 1..18: note = synthetic construct
  REGION 1..18: note = dimethylated H1.0K180me2 peptide
  MOD_RES 16: note = dimethyllysine
AKPVKASKPKKAKPVKPK

SEQ ID NO: 4 - protein, 17 aa, synthetic construct
  REGION 1..17: note = dimethylated H1.0K180me2 peptide
  MOD_RES 15: note = dimethyllysine
KPVKASKPKKAKPVKPK

SEQ ID NO: 5 - protein, 16 aa, synthetic construct
  REGION 1..16: note = dimethylated H1.0K180me2 peptide
  MOD_RES 14: note = dimethyllysine
PVKASKPKKAKPVKPK

SEQ ID NO: 6 - protein, 15 aa, synthetic construct
  REGION 1..15: note = dimethylated H1.0K180me2 peptide
  MOD_RES 13: note = dimethyllysine
VKASKPKKAKPVKPK

SEQ ID NO: 7 - protein, 14 aa, synthetic construct
  REGION 1..14: note = dimethylated H1.0K180me2 peptide
  MOD_RES 12: note = dimethyllysine
KASKPKKAKPVKPK

SEQ ID NO: 8 - protein, 13 aa, synthetic construct
  REGION 1..13: note = dimethylated H1.0K180me2 peptide
  MOD_RES 11: note = dimethyllysine
ASKPKKAKPVKPK

SEQ ID NO: 9 - protein, 12 aa, synthetic construct
  REGION 1..12: note = dimethylated H1.0K180me2 peptide
  MOD_RES 10: note = dimethyllysine
SKPKKAKPVKPK

SEQ ID NO: 10 - protein, 11 aa, synthetic construct
  REGION 1..11: note = dimethylated H1.0K180me2 peptide
  MOD_RES 9: note = dimethyllysine
KPKKAKPVKPK

SEQ ID NO: 11 - protein, 10 aa, synthetic construct
  REGION 1..10: note = dimethylated H1.0K180me2 peptide
  MOD_RES 8: note = dimethyllysine
PKKAKPVKPK

SEQ ID NO: 12 - protein, 9 aa, synthetic construct
  REGION 1..9: note = dimethylated H1.0K180me2 peptide
  MOD_RES 7: note = dimethyllysine
KKAKPVKPK

SEQ ID NO: 13 - protein, 8 aa, synthetic construct
  REGION 1..8: note = dimethylated H1.0K180me2 peptide
  MOD_RES 6: note = dimethyllysine
KAKPVKPK

SEQ ID NO: 14 - protein, 17 aa, synthetic construct
  REGION 1..17: note = dimethylated H1.0K180me2 peptide
  MOD_RES 16: note = dimethyllysine
AKPVKASKPKKAKPVKP

SEQ ID NO: 15 - protein, 16 aa, synthetic construct
  REGION 1..16: note = dimethylated H1.0K180me2 peptide
  MOD_RES 15: note = dimethyllysine
KPVKASKPKKAKPVKP

SEQ ID NO: 16 - protein, 15 aa, synthetic construct
  REGION 1..15: note = dimethylated H1.0K180me2 peptide
  MOD_RES 14: note = dimethyllysine
PVKASKPKKAKPVKP

SEQ ID NO: 17 - protein, 14 aa, synthetic construct
  REGION 1..14: note = dimethylated H1.0K180me2 peptide
  MOD_RES 13: note = dimethyllysine
VKASKPKKAKPVKP

SEQ ID NO: 18 - protein, 13 aa, synthetic construct
  REGION 1..13: note = dimethylated H1.0K180me2 peptide
  MOD_RES 12: note = dimethyllysine
KASKPKKAKPVKP

SEQ ID NO: 19 - protein, 12 aa, synthetic construct
  REGION 1..12: note = dimethylated H1.0K180me2 peptide
  MOD_RES 11: note = dimethyllysine
ASKPKKAKPVKP

SEQ ID NO: 20 - protein, 11 aa, synthetic construct
  REGION 1..11: note = dimethylated H1.0K180me2 peptide
  MOD_RES 10: note = dimethyllysine
SKPKKAKPVKP

SEQ ID NO: 21 - protein, 10 aa, synthetic construct
  REGION 1..10: note = dimethylated H1.0K180me2 peptide
  MOD_RES 9: note = dimethyllysine
KPKKAKPVKP

SEQ ID NO: 22 - protein, 9 aa, synthetic construct
  REGION 1..9: note = dimethylated H1.0K180me2 peptide
  MOD_RES 8: note = dimethyllysine
PKKAKPVKP

SEQ ID NO: 23 - protein, 8 aa, synthetic construct
  REGION 1..8: note = dimethylated H1.0K180me2 peptide
  MOD_RES 7: note = dimethyllysine
KKAKPVKP

SEQ ID NO: 24 - protein, 7 aa, synthetic construct
  REGION 1..7: note = dimethylated H1.0K180me2 peptide
  MOD_RES 6: note = dimethyllysine
KAKPVKP

SEQ ID NO: 25 - protein, 16 aa, synthetic construct
  REGION 1..16: note = dimethylated H1.0K180me2 peptide
  MOD_RES 16: note = dimethyllysine
AKPVKASKPKKAKPVK

SEQ ID NO: 26 - protein, 15 aa, synthetic construct
  REGION 1..15: note = dimethylated H1.0K180me2 peptide
  MOD_RES 15: note = dimethyllysine
KPVKASKPKKAKPVK

SEQ ID NO: 27 - protein, 14 aa, synthetic construct
  REGION 1..14: note = dimethylated H1.0K180me2 peptide
  MOD_RES 14: note = dimethyllysine
PVKASKPKKAKPVK

SEQ ID NO: 28 - protein, 13 aa, synthetic construct
  REGION 1..13: note = dimethylated H1.0K180me2 peptide
  MOD_RES 13: note = dimethyllysine
VKASKPKKAKPVK

SEQ ID NO: 29 - protein, 12 aa, synthetic construct
  REGION 1..12: note = dimethylated H1.0K180me2 peptide
  MOD_RES 12: note = dimethyllysine
KASKPKKAKPVK

SEQ ID NO: 30 - protein, 11 aa, synthetic construct
  REGION 1..11: note = dimethylated H1.0K180me2 peptide
  MOD_RES 11: note = dimethyllysine
ASKPKKAKPVK

SEQ ID NO: 31 - protein, 10 aa, synthetic construct
  REGION 1..10: note = dimethylated H1.0K180me2 peptide
  MOD_RES 10: note = dimethyllysine
SKPKKAKPVK

SEQ ID NO: 32 - protein, 9 aa, synthetic construct
  REGION 1..9: note = dimethylated H1.0K180me2 peptide
  MOD_RES 9: note = dimethyllysine
KPKKAKPVK

SEQ ID NO: 33 - protein, 8 aa, synthetic construct
  REGION 1..8: note = dimethylated H1.0K180me2 peptide
  MOD_RES 8: note = dimethyllysine
PKKAKPVK

SEQ ID NO: 34 - protein, 7 aa, synthetic construct
  REGION 1..7: note = dimethylated H1.0K180me2 peptide
  MOD_RES 7: note = dimethyllysine
KKAKPVK

SEQ ID NO: 35 - protein, 6 aa, synthetic construct
  REGION 1..6: note = dimethylated H1.0K180me2 peptide
  MOD_RES 6: note = dimethyllysine
KAKPVK

SEQ ID NO: 36 - protein, 19 aa, synthetic construct
  REGION 1..19: note = H1.0K180me2 peptide
CAKPVKASKPKKAKPVKPK

SEQ ID NO: 37 - protein, 20 aa, synthetic construct
  REGION 1..20: note = H1.0K180me2 peptide
CAKPVKASKPKKAKPVKPKC

SEQ ID NO: 38 - protein, 19 aa, synthetic construct
  REGION 1..19: note = H1.0K180me2 peptide
AKPVKASKPKKAKPVKPKC

SEQ ID NO: 39 - protein, 19 aa, synthetic construct
  REGION 1..19: note = H1.0K180me2 peptide
  MOD_RES 17: note = dimethyllysine
CAKPVKASKPKKAKPVKPK

SEQ ID NO: 40 - protein, 20 aa, synthetic construct
  REGION 1..20: note = H1.0K180me2 peptide
  MOD_RES 17: note = dimethyllysine
CAKPVKASKPKKAKPVKPKC

SEQ ID NO: 41 - protein, 19 aa, synthetic construct
  REGION 1..19: note = H1.0K180me2 peptide
  MOD_RES 16: note = dimethyllysine
AKPVKASKPKKAKPVKPKC

SEQ ID NO: 42 - protein, 18 aa, Homo sapiens
AKPVKASKPKKAKPVKPK

SEQ ID NO: 43 - protein, 17 aa, Homo sapiens
KPVKASKPKKAKPVKPK

SEQ ID NO: 44 - protein, 16 aa, Homo sapiens
PVKASKPKKAKPVKPK

SEQ ID NO: 45 - protein, 15 aa, Homo sapiens
VKASKPKKAKPVKPK

SEQ ID NO: 46 - protein, 14 aa, Homo sapiens
KASKPKKAKPVKPK

SEQ ID NO: 47 - protein, 13 aa, Homo sapiens
ASKPKKAKPVKPK

SEQ ID NO: 48 - protein, 12 aa, Homo sapiens
SKPKKAKPVKPK

SEQ ID NO: 49 - protein, 11 aa, Homo sapiens
KPKKAKPVKPK

SEQ ID NO: 50 - protein, 10 aa, Homo sapiens
PKKAKPVKPK

SEQ ID NO: 51 - protein, 9 aa, Homo sapiens
KKAKPVKPK

SEQ ID NO: 52 - protein, 8 aa, Homo sapiens
KAKPVKPK

SEQ ID NO: 53 - protein, 17 aa, Homo sapiens
AKPVKASKPKKAKPVKP

SEQ ID NO: 54 - protein, 16 aa, Homo sapiens
KPVKASKPKKAKPVKP

SEQ ID NO: 55 - protein, 15 aa, Homo sapiens
PVKASKPKKAKPVKP

SEQ ID NO: 56 - protein, 14 aa, Homo sapiens
VKASKPKKAKPVKP

SEQ ID NO: 57 - protein, 13 aa, Homo sapiens
KASKPKKAKPVKP

SEQ ID NO: 58 - protein, 12 aa, Homo sapiens
ASKPKKAKPVKP

SEQ ID NO: 59 - protein, 11 aa, Homo sapiens
SKPKKAKPVKP

SEQ ID NO: 60 - protein, 10 aa, Homo sapiens
KPKKAKPVKP

SEQ ID NO: 61 - protein, 9 aa, Homo sapiens
PKKAKPVKP

SEQ ID NO: 62 - protein, 8 aa, Homo sapiens
KKAKPVKP

SEQ ID NO: 63 - protein, 7 aa, Homo sapiens
KAKPVKP

SEQ ID NO: 64 - protein, 16 aa, Homo sapiens
AKPVKASKPKKAKPVK

SEQ ID NO: 65 - protein, 15 aa, Homo sapiens
KPVKASKPKKAKPVK

SEQ ID NO: 66 - protein, 14 aa, Homo sapiens
PVKASKPKKAKPVK

SEQ ID NO: 67 - protein, 13 aa, Homo sapiens
VKASKPKKAKPVK

SEQ ID NO: 68 - protein, 12 aa, Homo sapiens
KASKPKKAKPVK

SEQ ID NO: 69 - protein, 11 aa, Homo sapiens
ASKPKKAKPVK

SEQ ID NO: 70 - protein, 10 aa, Homo sapiens
SKPKKAKPVK

SEQ ID NO: 71 - protein, 9 aa, Homo sapiens
KPKKAKPVK

SEQ ID NO: 72 - protein, 8 aa, Homo sapiens
PKKAKPVK

SEQ ID NO: 73 - protein, 7 aa, Homo sapiens
KKAKPVK

SEQ ID NO: 74 - protein, 6 aa, Homo sapiens
KAKPVK

SEQ ID NO: 75 - protein, 18 aa, synthetic construct
  REGION 1..18: note = Biotin conjugated H1.0 peptide
  SITE 1: note = Ala is biotinylated
AKPVKASKPKKAKPVKPK

SEQ ID NO: 76 - protein, 18 aa, synthetic construct
  REGION 1..18: note = Biotin conjugated H1.0 dimethylated peptide
  SITE 1: note = Ala is biotinylated
  MOD_RES 16: note = dimethyllysine
AKPVKASKPKKAKPVKPK

SEQ ID NO: 77 - protein, 19 aa, synthetic construct
  REGION 1..19: note = KLH conjugated H1.0K180me2 peptide
  SITE 1: note = Cys is conjugated to Keyhole Limpet Hemocyanin
  MOD_RES 17: note = dimethyllysine
CAKPVKASKPKKAKPVKPK

SEQ ID NO: 78 - protein, 19 aa, synthetic construct
  REGION 1..19: note = Biotin conjugated H1.0K180me2 peptide
  SITE 1: note = Cys is biotinylated
  MOD_RES 17: note = dimethyllysine
CAKPVKASKPKKAKPVKPK

SEQ ID NO: 79 - protein, 19 aa, synthetic construct
  REGION 1..19: note = bovine serum albumin (BSA) conjugated H1.0 dimethylated peptide
  SITE 1: note = Cys is conjugated to bovine serum albumin (BSA)
  MOD_RES 17: note = dimethyllysine
CAKPVKASKPKKAKPVKPK

SEQ ID NO: 80 - DNA, 20 bp, synthetic construct
  misc_feature 1..20: note = GLP-Reverse primer
  source 1..20: mol_type = other DNA
gggatctttactggctgcat

SEQ ID NO: 81 - DNA, 20 bp, synthetic construct
  misc_feature 1..20: note = Beta-actin-Forward primer
  source 1..20: mol_type = other DNA
ctcttccagccttccttcct

SEQ ID NO: 82 - DNA, 20 bp, synthetic construct
  misc_feature 1..20: note = Beta-actin-Reverse primer
  source 1..20: mol_type = other DNA
agcactgtgttggcgtacag

SEQ ID NO: 83 - DNA, 20 bp, synthetic construct
  misc_feature 1..20: note = H1.0-Forward primer
  source 1..20: mol_type = other DNA
ctcaagcagaccaaaggggt

SEQ ID NO: 84 - DNA, 20 bp, synthetic construct
  misc_feature 1..20: note = H1.0-Reverse primer
  source 1..20: mol_type = other DNA
ggcgtggctaccttcttgat

SEQ ID NO: 85 - DNA, 20 bp, synthetic construct
  misc_feature 1..20: note = H1.1-Forward primer
  source 1..20: mol_type = other DNA
aggcaacgggtgcatctaaa

SEQ ID NO: 86 - DNA, 20 bp, synthetic construct
  misc_feature 1..20: note = H1.1-Reverse primer
  source 1..20: mol_type = other DNA
gatttccttgttgccgcagg

SEQ ID NO: 87 - DNA, 20 bp, synthetic construct
  misc_feature 1..20: note = H1.2-Forward primer
  source 1..20: mol_type = other DNA
caaagaaggccaaggttgcg

SEQ ID NO: 88 - DNA, 21 bp, synthetic construct
  misc_feature 1..21: note = H1.2-Reverse primer
  source 1..21: mol_type = other DNA
cgccttcttaggcttgacaac

SEQ ID NO: 89 - DNA, 20 bp, synthetic construct
  misc_feature 1..20: note = H1.3-Forward primer
  source 1..20: mol_type = other DNA
agtggccaagagtgcgaaaa

SEQ ID NO: 90 - DNA, 20 bp, synthetic construct
  misc_feature 1..20: note = H1.3-Reverse primer
  source 1..20: mol_type = other DNA
cttcggcttccccgacttag

SEQ ID NO: 91 - DNA, 21 bp, synthetic construct
  misc_feature 1..21: note = H1.4-Forward primer
  source 1..21: mol_type = other DNA
acgcttgccttcaacatgtcc

SEQ ID NO: 92 - DNA, 20 bp, synthetic construct
  misc_feature 1..20: note = H1.4-Reverse primer
  source 1..20: mol_type = other DNA
agtaatgagctcggacaccg

SEQ ID NO: 93 - DNA, 20 bp, synthetic construct
  misc_feature 1..20: note = H1.5-Forward primer
  source 1..20: mol_type = other DNA
ccggctaagaagaaggcaac

SEQ ID NO: 94 - DNA, 20 bp, synthetic construct
  misc_feature 1..20: note = H1.5-Reverse primer
  source 1..20: mol_type = other DNA
gctccttagaagcagccaca

SEQ ID NO: 95 - DNA, 20 bp, synthetic construct
  misc_feature 1..20: note = G9A-Forward primer
  source 1..20: mol_type = other DNA
tgctgaggctgatgtgagag

SEQ ID NO: 96 - DNA, 20 bp, synthetic construct
  misc_feature 1..20: note = G9A-Reverse primer
  source 1..20: mol_type = other DNA
ggtcacacaggtggttgatg

SEQ ID NO: 97 - protein, 21 aa, synthetic construct
  REGION 1..21: note = H10K180 peptide
ASKPKKAKPVKPKAKSSAKRA

SEQ ID NO: 98 - protein, 21 aa, synthetic construct
  REGION 1..21: note = H10K177 peptide
PVKASKPKKAKPVKPKAKSSA

SEQ ID NO: 99 - protein, 21 aa, synthetic construct
  REGION 1..21: note = H10K175 peptide
AKPVKASKPKKAKPVKPKAKS

SEQ ID NO: 100 - protein, 21 aa, synthetic construct
  REGION 1..21: note = H10K174 peptide
KAKPVKASKPKKAKPVKPKAK

SEQ ID NO: 101 - protein, 21 aa, synthetic construct
  REGION 1..21: note = H10K172 peptide
TVKAKPVKASKPKKAKPVKPK

SEQ ID NO: 102 - protein, 14 aa, synthetic construct
  REGION 1..14: note = H3K4 peptide
ARTKQTARKSTGGK

SEQ ID NO: 103 - protein, 19 aa, synthetic construct
  REGION 1..19: note = H3K9 peptide
ARTKQTARKSTGGKAPRKQ

SEQ ID NO: 104 - protein, 21 aa, synthetic construct
  REGION 1..21: note = H3K27 peptide
RKQLATKVARKSAPATGGVKK

SEQ ID NO: 105 - protein, 21 aa, synthetic construct
  REGION 1..21: note = H3K36 peptide
RKSAPATGGVKKPHRYRPGTV

SEQ ID NO: 106 - protein, 21 aa, synthetic construct
  REGION 1..21: note = H3K79 peptide
RLMREIAQDFKTDLRFQSSAV

SEQ ID NO: 107 - DNA, 20 bp, synthetic construct
  misc_feature 1..20: note = GLP-Forward primer
  source 1..20: mol_type = other DNA
aggggagtgctgacacagag

SEQ ID NO: 108 - protein, 215 aa, synthetic construct
  REGION 1..215: note = H1.1 protein
MSETVPPAPAASAAPEKPLAGKKAKKPAKAAAASKKKPAGPSVSELIVQAASSSKERGGVSLAALKKALAAAGYDVEKNNSRIKLGIKSLVSKGTLVQTKGTGASGSFKLNKKASSVETKPGASKVATKTKATGASKKLKKATGASKKSVKTPKKAKKPAATRKSSKNPKKPKTVKPKKVAKSPAKAKAVKPKAAKARVTKPKTAKPKKAAPKKK

SEQ ID NO: 109 - protein, 213 aa, synthetic construct [sequence omitted]

SEQ ID NO: 110 - protein, 221 aa, synthetic construct
  REGION 1..221: note = H1.3 protein
MSETAPLAPTIPAPAEKTPVKKKAKKAGATAGKRKASGPPVSELITKAVAASKERSGVSLAALKKALAAAGYDVEKNNSRIKLGLKSLVSKGTLVQTKGTGASGSFKLNKKAASGEGKPKAKKAGAAKPRKPAGAAKKPKKVAGAATPKKSIKKTPKKVKKPATAAGTKKVAKSAKKVKTPQPKKAAKSPAKAKAPKPKAAKPKSGKPKVTKAKKAAPKKK

SEQ ID NO: 111 - protein, 219 aa, synthetic construct
  REGION 1..219: note = H1.4 protein
MSETAPAAPAAPAPAEKTPVKKKARKSAGAAKRKASGPPVSELITKAVAASKERSGVSLAALKKALAAAGYDVEKNNSRIKLGLKSLVSKGTLVQTKGTGASGSFKLNKKAASGEAKPKAKKAGAAKAKKPAGAAKKPKKATGAATPKKSAKKTPKKAKKPAAAAGAKKAKSPKKAKAAKPKKAPKSPAKAKAVKPKAAKPKTAKPKAAKPKKAAAKKK

SEQ ID NO: 112 - protein, 226 aa, synthetic construct
  REGION 1..226: note = H1.5 protein
MSETAPAETATPAPVEKSPAKKKATKKAAGAGAAKRKATGPPVSELITKAVAASKERNGLSLAALKKALAAGGYDVEKNNSRIKLGLKSLVSKGTLVQTKGTGASGSFKLNKKAASGEAKPKAKKAGAAKAKKPAGATPKKAKKAAGAKKAVKKTPKKAKKPAAAGVKKVAKSPKKAKAAAKPKKATKSPAKPKAVKPKAAKPKAAKPKAAKPKAAKAKKAAAKKK

SEQ ID NO: 113 - protein, 213 aa, synthetic construct
  REGION 1..213: note = H1.X protein
MSVELEEALPVTTAEGMAKKVTKAGGSAALSPSKKRKNSKKKNQPGKYSQLVVETIRRLGERNGSSLAKIYTEAKKVPWFDQQNGRTYLKYSIKALVQNDTLLQVKGTGANGSFKLNRKKLEGGGERRGAPAAATAPAPTAHKAKKAAPGAAGSRRADKKPARGQKPEQRSHKKGAGAKKDKGGKAKKTAAAGGKKVKKAAKPSVPKVPKGRK

SEQ ID NO: 114 - protein, 18 aa, synthetic construct
  REGION 1..18: note = dimethylated K180me2 peptide
  MOD_RES 2: note = methyllysine
  MOD_RES 16: note = dimethyllysine
AKPVKASKPKKAKPVKPK

SEQ ID NO: 115 - protein, 18 aa, synthetic construct
  REGION 1..18: note = dimethylated K180me2 peptide
  MOD_RES 10..11: note = trimethyllysine
  MOD_RES 13: note = methyllysine
  MOD_RES 16: note = dimethyllysine
AKPVKASKPKKAKPVKPK

SEQ ID NO: 116 - protein, 18 aa, synthetic construct
  REGION 1..18: note = dimethylated K180me2 peptide
  MOD_RES 10: note = methyllysine
  MOD_RES 16: note = trimethyllysine
AKPVKASKPKKAKPVKPK

SEQ ID NO: 117 - protein, 194 aa, synthetic construct; identical to SEQ ID NO: 1
  REGION 1..194: note = methylated H1.0 protein
  MOD_RES 121: note = dimethyllysine
  MOD_RES 169: note = dimethyllysine
  MOD_RES 172: note = methyllysine
  MOD_RES 175: note = dimethyllysine
  MOD_RES 180: note = dimethyllysine

SEQ ID NO: 118 - protein, 194 aa, synthetic construct; identical to SEQ ID NO: 1
  REGION 1..194: note = methylated H1.0 protein
  MOD_RES 111: note = dimethyllysine
  MOD_RES 112: note = trimethyllysine
  MOD_RES 115: note = dimethyllysine
  MOD_RES 116: note = trimethyllysine
  MOD_RES 121: note = trimethyllysine
  MOD_RES 122: note = dimethyllysine
  MOD_RES 125: note = methyllysine
  MOD_RES 127: note = dimethyllysine
  MOD_RES 128: note = trimethyllysine
  MOD_RES 137: note = trimethyllysine
  MOD_RES 139: note = dimethyllysine
  MOD_RES 144: note = methyllysine
  MOD_RES 145: note = trimethyllysine
  MOD_RES 147: note = dimethyllysine
  MOD_RES 148: note = trimethyllysine
  MOD_RES 149: note = methyllysine
  MOD_RES 155..156: note = trimethyllysine
  MOD_RES 158: note = dimethyllysine
  MOD_RES 159: note = methyllysine
  MOD_RES 161: note = trimethyllysine
  MOD_RES 164: note = trimethyllysine
  MOD_RES 166: note = dimethyllysine
  MOD_RES 169: note = trimethyllysine
  MOD_RES 172: note = methyllysine
  MOD_RES 174..175: note = trimethyllysine
  MOD_RES 177: note = dimethyllysine
  MOD_RES 180: note = methyllysine
  MOD_RES 182: note = methyllysine
  MOD_RES 184: note = trimethyllysine
  MOD_RES 188: note = dimethyllysine
  MOD_RES 192: note = dimethyllysine

(19) United States
(12) Patent Application Publication
(10) Pub. No.: US 2024/0255509 A1
(43) Pub. Date: Aug. 1, 2024

(54) COMPOSITIONS AND METHODS RELATED TO K180 DIMETHYLATED H1.0 PROTEIN

(71) Applicant: Aelan Cell Technologies, Inc., San Francisco, CA (US)

(72) Inventors: Victoria LUNYAK, San Anselmo, CA (US); James Robert TOLLERVEY, San Rafael, CA (US)

(21) Appl. No.: 18/506,086

(22) Filed: Nov. 9, 2023

Related U.S. Application Data
(62) Division of application No. 16/164,676, filed on Oct. 18, 2018, now abandoned.
Application No. 16/164,676 is a continuation of application No. PCT/US2017/028686, filed on Apr. 20, 2017.
(60) Provisional application No. 62/325,392, filed on Apr. 20, 2016.
(60) Provisional application No. 62/325,408, filed on Apr. 20, 2016.
(60) Provisional application No. 62/325,362, filed on Apr. 20, 2016.
(60) Provisional application No. 62/355,265, filed on Jun. 27, 2016.
(60) Provisional application No. 62/355,277, filed on Jun. 27, 2016.

(51) Int. Cl. G01N 33/573; C07K 14/47; C07K 16/18; C12N 9/10; C12P 21/02; C12P 21/06; G01N 33/543; G01N 33/68

(52) U.S. Cl. CPC G01N 33/573; C07K 14/47; C07K 16/18; C12N 9/1007; C12P 21/02; C12P 21/06; C12Y 201/01043; G01N 33/54306; G01N 33/6896; C12Y 201/01; G01N 2800/2821; G01N 2800/50

(57) ABSTRACT

Provided herein are H1.0K180me2 antibodies, H1.0K180me2 proteins, and H1.0K180me2 peptides and methods of use for diagnostics and therapeutics. These H1.0K180me2 antibodies, H1.0K180me2 proteins, and H1.0K180me2 peptides may be used in the treatment of methylated H1.0-related diseases or conditions in an individual. These H1.0K180me2 antibodies, H1.0K180me2 proteins, and H1.0K180me2 peptides may also be used for the detection and quantification of a histone H1.0 protein or fragment thereof, comprising a dimethylated lysine at lysine residue 180 (H1.0K180me2); such compositions and methods are useful for detecting replicative senescence, DNA damage, genotoxic stress, radiation exposure, Alzheimer's disease, are useful for monitoring therapeutic regimens, patient stratification, drug screening, and may serve as a marker of biological aging in a system.

### CROSS-REFERENCE TO RELATED APPLICATIONS

This application is a division of U.S. patent application Ser. No. 16/164,676, filed Oct. 18, 2018, which is a continuation of International Patent Application No. PCT/US2017/028686, filed Apr. 20, 2017, which claims priority to U.S. Provisional Application Ser. No. 62/325,392, filed Apr. 20, 2016, U.S. Provisional Application Ser. No. 62/325,362, filed Apr. 20, 2016, U.S. Provisional Application Ser. No. 62/325,408, filed on Apr. 20, 2016, U.S. Provisional Application Ser. No. 62/355,265, filed Jun. 27, 2016, and U.S. Provisional Application Ser. No. 62/355,277, filed Jun. 27, 2016, each of which are incorporated herein by reference in their entirety.

### DESCRIPTION OF THE XML FILE SUBMITTED ELECTRONICALLY

The contents of the XML file submitted electronically herewith are incorporated herein by reference in their entirety: A computer readable format copy of the Sequence Listing (file name: ALNC_004_03US_SeqList_ST26.xml, date of creation: Nov. 7, 2023, file size: 171,669 bytes).

### BACKGROUND

Cellular chromatin is a dynamic polymer, capable of many configurations, and prone to remodeling and restructuring as it receives physiologically relevant input. Histone proteins are the main protein components of chromatin and double-stranded DNA is wound around histone proteins. Changes in histone proteins can differentially alter access of the transcriptional machinery to some genes while leaving access to other genes intact. Differential chromatin condensation achieved by histone posttranslational modifications (PTMs) underlies packaging of chromatin (Lunyak and Rosenfeld 92008) Hum. Mol. Genet., 17: R28-36; Jenuwein and Allis (2001) Science, 293: 1074-1080). Histone PTMs, for example methylation, can act as an epigenetic code and play critical roles in many aspects of the cellular responses tightly linked to development, injury, disease and aging.

Five major families of histones exist: H1, H2A, H2B, H3 and H4. Histones H2A, H2B, H3 and H4 are known as the core histones, while histones H1 and H5 are known as the linker histones. The large number of H1.0-binding proteins identified by multiple studies in recent years point to an important role for protein-protein interactions of H1.0 and suggests a new paradigm for H1.0 structure and function that extends beyond its effects on chromatin architecture.

There is a need to detect H1.0 methylation in various cellular contexts; and sensitive assays are needed to differentiate among the various types of cellular contexts, as they relate to the development of disease, response to injury, and response to therapeutic regimens. There is also a need to therapeutically address methylated H1.0-related diseases and conditions. Provided herein are methods and compositions for this purpose.

### BRIEF SUMMARY

Provided herein are H1.0K180me2 antibodies, H1.0K180me2 proteins, and H1.0K180me2 peptides and methods of use for diagnostics and therapeutics. These H1.0K180me2 antibodies, H1.0K180me2 proteins, and H1.0K180me2 peptides may be used in the treatment of methylated H1.0-related diseases or conditions in an individual. These H1.0K180me2 antibodies, H1.0K180me2 proteins, and H1.0K180me2 peptides may also be used for the detection and quantification of a histone H1.0 protein or fragment thereof, comprising a dimethylated lysine at lysine residue 180 (H1.0K180me2); such compositions and methods are useful for detecting replicative senescence, DNA damage, genotoxic stress, radiation exposure, Alzheimer's disease, are useful for monitoring therapeutic regimens, patient stratification, drug screening, and may serve as a marker of biological aging in a system.

In one aspect, provided herein is an antibody that specifically binds a dimethylated antigen, wherein the dimethylated antigen comprises dimethylated lysine residue, wherein the lysine residue corresponds to K180 of a human histone H1.0, and wherein the dimethylated lysine residue is required for binding (H1.0K180me2 antibodies). In some embodiments, the dimethylated antigen does not comprise any other lysine residues that are methylated. In some embodiments, the antibody does not bind, or only minimally binds, if antigen comprises dimethylated lysine residues at lysine residues corresponding to K166, K172, K174, K175, and/or K177 of a human histone H1.0 protein. In some embodiments, the antibody does not bind, or only minimally binds, antigen comprises monomethylated lysine residues at lysine residues corresponding to K166, K172, K174, K175, K177, and/or K180 of a human histone H1.0 protein. In some embodiments, the antibody does not bind, or only minimally binds, if the antigen comprises trimethylated lysine residues at lysine residues corresponding to K166, K172, K174, K175, K177, and/or K180 of a human histone H1.0 protein. In some embodiments, the antibody is at least 2-fold more specific for the dimethylated antigen, than a monomethylated antigen, wherein the monomethylated antigen comprises a monomethylated lysine residue, and wherein the lysine residue corresponds to K180 of a human histone H1.0 protein. In some embodiments, the antibody is a monoclonal antibody. In some embodiments, the antibody is a polyclonal antibody. In some embodiments, the antibody is labeled. In some embodiments, the antibody is biotinylated. In some embodiments, the antibody is attached to solid surface. In some embodiments, the antibody is attached a bead, column, resin, or a microplate. In some embodiments, the antibody is a cell penetrating antibody. In some embodiments, the antibody is an IgG1 antibody. In some embodiments, the antibody is a human antibody. In some embodiments, the antibody is a humanized antibody. In some embodiments, the antibody is conjugated to at least one therapeutic agent selected from the group consisting of a radionuclide, a cytotoxin, a chemotherapeutic agent, a drug, a pro-drug, a toxin, an enzyme, an immunomodulator, a pro-apoptotic agent, a cytokine, a hormone, an oligonucleotide, an antisense molecule, a siRNA, and a second antibody. In some embodiments, the antibody is capable of clearing H1.0K180me2 in a sample. In some embodiments, the antibody is capable of clearing cells comprising H1.0K180me2. In some embodiments, the antibody is capable of clearing senescent cells.

In another aspect provided herein is a method of treating a methylated H1.0-related disease or condition in an individual comprising administering to the individual a therapeutically effective amount of any one of the H1.0K180me2 antibodies provided herein. In some embodiments, the methylated H1.0-related disease or condition is selected from the group consisting of Alzheimer's disease, radiation exposure, exposure to a genotoxic stressor, a disease or condition comprising the accumulation of senescent cells, and a disease or condition accompanied by elevated levels of H1.0K180me2 proteins or peptides. In another related aspect, provided herein is a method of clearing H1.0K180me2 in an individual comprising administering to the individual a therapeutically effective amount of an antibody of any one of the H1.0K180me2 antibodies provided herein, for example for treating an individual who is suffering from a disease or condition selected from the group consisting of Alzheimer's disease, radiation exposure, exposure to a genotoxin, exposure to a DNA damaging agent, and a condition comprising the accumulation of senescent cells. In a related aspect, provided herein are pharmaceutical compositions, kits, and other articles of manufacture comprising any one of the H1.0K180me2 antibodies described herein.

In another aspect, provided herein is a histone H1.0 peptide comprising a dimethylated lysine residue, or a histone H1.0 protein comprising a dimethylated lysine residue, wherein the dimethylated lysine residue corresponds to K180 of a human histone H1.0 (H1.0K180me2 peptide and H1.0K180me2 protein). In some embodiments, the peptide comprises the sequence selected from the group consisting of SEQ ID NOS:3-35. In some embodiments, the peptide comprises the sequence selected from the group consisting of SEQ ID NOS:3-5. In some embodiments, the protein comprises the sequence of SEQ ID NO:2. In some embodiments, the protein comprises the sequence of SEQ ID NO:3. In some embodiments, the peptide or protein does not comprise any other lysine residues that are methylated. In some embodiments, the peptide or protein is labeled. In some embodiments, the peptide or protein is biotinylated. In some embodiments, the peptide or protein is attached to solid surface. In some embodiments, the peptide or protein is attached a bead, column, resin, or a microplate. In some embodiments, the peptide or protein is conjugated to at least one therapeutic agent selected from the group consisting of a radionuclide, a cytotoxin, a chemotherapeutic agent, a drug, a pro-drug, a toxin, an enzyme, an immunomodulator, a pro-apoptotic agent, a cytokine, a hormone, an oligonucleotide, an antisense molecule, a siRNA, and a second antibody. In some embodiments, the peptide or protein is capable of clearing or blocking H1.0K180me2 autoantibodies. In some embodiments, the peptide or protein is capable of clearing or blocking H1.0K180me2 IgG autoantibodies. In some embodiments, the peptide or protein is capable of clearing or blocking H1.0K180me2 IgM autoantibodies.

In another aspect, provided herein is a method of treating a methylated H1.0-related disease or condition in an individual comprising administering to the individual a therapeutically effective amount of any one of the H1.0K180me2 proteins or H1.0K180me2 peptides described herein. In some embodiments, the methylated H1.0-related disease or condition is selected from the group consisting of Alzheimer's disease, radiation exposure, exposure to a genotoxic stressor, a disease or condition comprising the accumulation of senescent cells, and a disease or condition accompanied by elevated levels of H1.0K180me2 autoantibodies. In a related aspect, provided herein is a method of clearing, blocking, or neutralizing H1.0K180me2 autoantibodies in an individual comprising administering to the individual a therapeutically effective amount of any one of the H1.0K180me2 proteins or H1.0K180me2 peptides described herein. In some embodiments, the individual suffers from a disease or condition selected from the group consisting of Alzheimer's disease, radiation exposure, exposure to a genotoxic stressor, a disease or condition comprising the accumulation of senescent cells, or a disease or condition accompanied by elevated levels of H1.0K180me2 autoantibodies. In a related aspect, provided herein are pharmaceutical compositions, kits, and other articles of manufacture comprising any one of the H1.0K180me2 peptides or proteins described herein.

In another aspect, provided herein is a method of determining whether an individual has, or is at risk of developing, Alzheimer's disease, comprising: (a) contacting a biological sample from the individual with any H1.0K180me2 antibody provided herein; and (b) determining the concentration of the H1.0K180me2 antigen in the sample that binds the antibody, wherein a decrease in the concentration relative to a control indicates that the individual has, or is at risk of developing, Alzheimer's disease. In one embodiment, the individual has, or is at risk of developing, Alzheimer's disease when the serum concentration of the histone H1.0 protein is less than or below 5.61 nmol/ml. In some embodiments, the individual has, or is at risk of developing, Alzheimer's disease when the PLR (LR+) is 3.6 or greater. In some embodiments, the individual does not have a risk of developing, Alzheimer's disease when the NLR (LR−) is 0.26 or lower. In some embodiments, the method further comprises treating the individual with an Alzheimer's disease drug or regimen if it is determined that the individual has, or is at risk of developing, Alzheimer's disease. Accordingly, provided herein is a method of treating an individual with an Alzheimer's disease drug or regimen when the serum concentration of the histone H1.0 protein is less than or below 5.61 nmol/ml and/or when the measure PLR (LR+) is 3.6, as determined by the above method.

In another aspect, provided herein is a method of determining whether an individual has, or is at risk of developing, Alzheimer's disease, comprising: (a) contacting a biological sample from the individual with an H1.0K180me2 protein or H1.0K180me2 peptide provided herein; and (b) determining the concentration of autoantibodies in the sample that bind the protein or peptide, wherein an increase in the concentration relative to a control indicates that the individual has, or is at risk of developing, Alzheimer's disease. In some embodiments, the method comprises determining the concentration of IgM autoantibodies in the sample that bind the protein or peptide. In some embodiments, the method comprises determining the concentration of IgG autoantibodies in the sample that bind the protein or peptide. In some embodiments, the individual has, or is at risk of developing, Alzheimer's disease when the serum concentration of the IgG autoantibodies is greater than or equal to 9.69 ug/ml, normalized to total IgG levels. In some embodiments, the individual has, or is at risk of developing, Alzheimer's disease when the serum concentration of the IgG autoantibodies is greater than or equal to 8.23 ug/ml, normalized to serum volume. In some embodiments, the individual has, or is at risk of developing, Alzheimer's disease when the serum concentration of the IgM autoantibodies is greater than or equal to 409 fMol/ml, normalized to serum volume. In some embodiments, the individual has, or is at risk of developing, Alzheimer's disease when the ratio of the concentration of IgM autoantibodies to total IgM concentration is greater than 26.6×10⁻⁶. In some embodiments, the individual has, or is at risk of developing, Alzheimer's disease when the PLR (LR+) is 2.4 or greater for IgG autoantibodies or the PLR (LR+) is 3.5 or greater for IgM autoantibodies. In some embodiments, the method comprises determining the concentration of IgM autoantibodies in the sample that bind the protein or peptide, and comparing that measurement to the total IgM concentration in the sample. In some embodiments, the method comprises treating the individual with an Alzheimer's disease drug or regimen if it is determined that the individual has, or is at risk of developing, Alzheimer's disease.

In another aspect, provided herein is a method of determining whether an individual diagnosed with Alzheimer's disease and receiving treatment for the Alzheimer's disease, will benefit from the treatment or will continue to benefit from the treatment, the method comprising: (a) contacting a biological sample from the individual with any H1.0K180me2 antibody provided herein; (b) determining the concentration of the H1.0K180me2 antigen in the sample that binds the antibody; and (c) determining that the individual will benefit from the treatment or will continue to benefit from the treatment if there is an increase in the concentration, relative to a control. In some embodiments, the method further comprises treating the individual with an Alzheimer's disease drug or regimen if it is determined that the individual will benefit or continue to benefit from the treatment.

In another aspect, provided herein is a method of determining whether an individual diagnosed with Alzheimer's disease and receiving treatment for the Alzheimer's disease, will benefit from the treatment or will continue to from the treatment, the method comprising: (a) contacting a biological sample from the individual with an H1.0K180me2 protein or peptide provided herein; (b) determining the concentration of autoantibodies in the sample that bind the protein or peptide; and (c) determining that the individual will benefit from the treatment or will continue to benefit from the treatment if there is an decrease in the concentration relative to a control. In some embodiments, the method comprises determining the concentration of IgM autoantibodies in the sample that bind the protein or peptide. In some embodiments, the method comprises determining the concentration of IgG autoantibodies in the sample that bind the protein or peptide.

In another aspect, provided herein is a method of determining whether an individual diagnosed with Alzheimer's disease will benefit from a candidate treatment, wherein the individual has not yet started the treatment, the method comprising: (a) administering to the individual a candidate treatment; (b) contacting a biological sample from the individual after administration of the candidate treatment with any H1.0K180me2 antibody provided herein; (c) determining the concentration and/or subcellular localization of the H1.0K180me2 antigen in the sample that binds the antibody; and (d) determining that the individual will benefit from the candidate treatment if there is an increase in the concentration or an decrease in cytoplasmic subcellular localization, relative to a control.

In another aspect, provided herein is a method of determining whether an individual diagnosed with Alzheimer's disease will benefit from a candidate treatment, wherein the individual has not yet started the treatment, the method comprising: (a) contacting a biological sample from the individual with a candidate treatment; (b) contacting the biological sample with any H1.0K180me2 antibody provided herein; (c) determining the concentration and/or subcellular localization of the H1.0K180me2 antigen in the sample that binds the antibody; and (d) determining that the individual will benefit from the candidate treatment if there is an increase in the concentration relative to a control or an decrease in cytoplasmic subcellular localization, relative to a control.

In another aspect, provided herein is a method of determining whether an individual diagnosed with Alzheimer's disease will benefit from a candidate treatment, wherein the individual has not yet started the treatment, the method comprising: (a) administering to the individual a candidate treatment; (b) contacting a biological sample from the individual with an H1.0K180me2 protein or H1.0K180me2 peptide provided herein; (c) determining the concentration of autoantibodies in the sample that bind the protein or peptide; and (d) determining that the individual will benefit from the candidate treatment if there is a decrease in the concentration, relative to a control.

As it pertains to methods related to Alzheimer's disease methods, in some embodiments, the change (increase, decrease, or no change) that is measured is relative to a threshold established by a Receiver Operating Characteristic curve analysis for optimal specificity and sensitivity. In some embodiments, the biological sample is selected from the group consisting of whole blood, plasma, serum, saliva, urine, feces, synovial fluid, cerebrospinal fluid, bronchial lavage, ascites fluid, bone marrow aspirate, pleural effusion, tissue, cells, a biopsy, interstitial fluid, and lymphatic fluid. In some embodiments, the individual is 60 years of age or older. In some embodiments utilizing an antibody, the antibody is labeled. In some embodiments, the H1.0K180me2 antibody is attached to a bead, column, resin, or a microplate. In some embodiments, the H1.0K180me2 protein or H1.0K180me2 peptide is labeled. In some embodiments, the H1.0K180me2 protein or H1.0K180me2 peptide is biotinylated. In some embodiments, the H1.0K180me2 protein or H1.0K180me2 peptide is attached to a bead, resin, column, or a microplate. In some embodiments, the H1.0K180me2 protein comprises the sequence of SEQ ID NO:2. In some embodiments, the H1.0K180me2 peptide comprises any one of the sequences of SEQ ID NOS:3-35. In some embodiments, the H1.0K180me2 peptide comprises the sequence of SEQ ID NO:3. In some embodiments, the concentration of autoantibodies specific to the H1.0K180me2 antigen is determined. In some embodiments, the concentration of H1.0K180me2 or H1.0K180me2 autoantibodies is determined via an ELISA. In some embodiments, the concentration of IgG autoantibodies is determined. In some embodiments, the concentration of IgM autoantibodies is determined. In some embodiments, the concentration of IgG and IgM autoantibodies is determined.

In the various aspects related to determining whether an individual diagnosed with Alzheimer's disease will benefit from a candidate treatment, and determining whether an individual diagnosed with Alzheimer's disease and receiving treatment for the Alzheimer's disease, or will benefit from the treatment or will continue to from the treatment, the treatment is selected from the group consisting of APP synthesis Inhibitors, beta-secretase inhibitors, gamma-secretase inhibitors and modulators, Aß aggregation inhibitors, Aß immunotherapy, Cholesterol-lowering drugs, Anti-tau drugs, cholinesterase inhibitors, N-methyl D-aspartate (NMDA) antagonists, atypical antipsychotics, blockers of protein S-nitrosylation, glucagon-like peptide-1 receptor agonists, rapamycin, rapalogues, endocannabinoids, cannabinoids, neuroprotectors, molecules controlling calcium influx, antioxidants, anti-inflammatory drugs, drugs controlling control of glutamate homeostasis, autophagy inducers, hormones, hormonal regulators, statins, insulin, insulin carriers, multifunctional nanocarriers, vitamins, nutritional supplements, small RNA molecules, peptides, and ultrasound therapy.

In another aspect, provided herein is a method of determining whether an individual has been exposed to a DNA damaging agent, comprising: (a) contacting a biological sample from the individual with any H1.0K180me2 antibody provided herein; and (b) determining the concentration of an H1.0K180me2 antigen in the sample that binds the antibody, wherein an increase in the concentration relative to a control indicates that the individual has been exposed to a DNA damaging agent. In some embodiments, the increase is above a threshold established by a Receiver Operating Characteristic curve analysis for optimal specificity and sensitivity. In some embodiments, the DNA damaging agent is radiation. In some embodiments, the biological sample is selected from the group consisting of whole blood, plasma, serum, saliva, urine, synovial fluid, cerebrospinal fluid, bronchial lavage, ascites fluid, bone marrow aspirate, pleural effusion, tissue, cells, a biopsy, interstitial fluid, and lymphatic fluid. In some embodiments, the antibody is labeled. In some embodiments, the antibody is attached to a bead, resin, column, or a microplate. In some embodiments, the concentration is determined via an ELISA. In some embodiments the antibody is at least 2-fold more specific for the dimethylated K180 antigen, than a monomethylated antigen, wherein the monomethylated antigen comprises a monomethylated lysine residue, and wherein the lysine residue corresponds to K180 of a human histone H1.0 protein. In some embodiments the antibody is at least 2-fold more specific for the dimethylated antigen, than a trimethylated antigen, wherein the trimethylated antigen comprises a trimethylated lysine residue, and wherein the lysine residue corresponds to K180 of a human histone H1.0 protein. In some embodiments the antibody is an antigen binding fragment.

In another aspect, provided herein is a method of determining whether an individual has been exposed to a DNA damaging agent, comprising: (a) contacting a biological sample from the individual with any H1.0K180me2 protein or H1.0K180me2 peptide provided herein; and (b) determining the concentration of autoantibodies in the sample that bind the protein or peptide, wherein an increase in the concentration relative to a control indicates that the individual has been exposed to a DNA damaging agent. In some embodiments, the increase is above a threshold established by a Receiver Operating Characteristic curve analysis for optimal specificity and sensitivity. In some embodiments, the DNA damaging agent is radiation. In some embodiments, the biological sample is selected from the group consisting of whole blood, plasma, serum, saliva, urine, synovial fluid, cerebrospinal fluid, bronchial lavage, ascites fluid, bone marrow aspirate, pleural effusion, tissue, cells, a biopsy, interstitial fluid, and lymphatic fluid. In some embodiments, the protein or peptide comprises a label. In some embodiments, the protein or peptide is attached to a bead, resin, column, or a microplate. In some embodiments, the concentration is determined via an ELISA. In some embodiments, the peptide comprises any one of the sequences of SEQ ID NOS:3-35. In some embodiments, the protein comprises the sequence of SEQ ID NO:2. In some embodiments, the peptide comprises the sequence of SEQ ID NO:3.

In another aspect, provided herein is a method of determining whether an individual receiving treatment with a rapalogue is responsive to such treatment, comprising: (a) contacting a biological sample from the individual with any H1.0K180me2 antibody provided herein; (b) determining the concentration of an H1.0K180me2 antigen in the sample that binds the antibody; and (c) determining whether the individual is responsive to treatment, wherein a decrease in the concentration relative to a control indicates that the individual is responsive. In some embodiments, the decrease is below a threshold established by a Receiver Operating Characteristic curve analysis for optimal specificity and sensitivity. In some embodiments, the rapalogue is selected from the group consisting of Rapamycin, Sirolimus, Rapamune, Everolimus, RA 001, Afinitor, Zortress, Temsirolimus, CCI-779, Torisel, Ridaforolimus, AP23573, MK-8669, Deforolimus, Zotarolimus, ABT-578, AZD8055, AZD2014, OSI-027, MLN0128, WYE-132, Torin1, PI-103, P7170, PF-04691502, PF-05212384, PKI-587, GNE477, PKI-180, WJD008, XL765, SAR245409, NVP-BEZ235, BGT226, SF1126, GSK2126458, Ku-0063794, WYE-354, NVP-BEZ235, PF-05212384, XL765, Torin 2, WYE-125132, and OSI-027. In some embodiments, the biological sample is selected from the group consisting of whole blood, plasma, serum, saliva, urine, synovial fluid, cerebrospinal fluid, bronchial lavage, ascites fluid, bone marrow aspirate, pleural effusion, tissue, cells, a biopsy, interstitial fluid, and lymphatic fluid. In some embodiments, the antibody is labeled. In some embodiments, the antibody is attached to a bead, resin, column, or a microplate. In some embodiments, the concentration is determined via an ELISA. In some embodiments, the antibody is an antigen binding fragment. In some embodiments the antibody is at least 2-fold more specific for the dimethylated K180 antigen, than a monomethylated antigen, wherein the monomethylated antigen comprises a monomethylated lysine residue, and wherein the lysine residue corresponds to K180 of a human histone H1.0 protein. In some embodiments the antibody is at least 2-fold more specific for the dimethylated antigen, than a trimethylated antigen, wherein the trimethylated antigen comprises a trimethylated lysine residue, and wherein the lysine residue corresponds to K180 of a human histone H1.0 protein.

In another aspect, provided herein is a method of determining whether an individual receiving treatment with a rapalogue is responsive to such treatment, comprising: (a) contacting a biological sample from the individual with an H1.0K180me2 peptide or H1.0K180me2 protein; (b) determining the concentration of autoantibodies in the sample that bind the protein or peptide; and (c) determining whether the individual is responsive to treatment, wherein a change in the concentration of autoantibodies relative to a control indicates that the individual is responsive. In some embodiments, the change is with respect to a threshold established by a Receiver Operating Characteristic curve analysis for optimal specificity and sensitivity. In some embodiments, the rapalogue is selected from the group consisting of Rapamycin, Sirolimus, Rapamune, Everolimus, RA 001, Afinitor, Zortress, Temsirolimus, CCI-779, Torisel, Ridaforolimus, AP23573, MK-8669, Deforolimus, Zotarolimus, ABT-578, AZD8055, AZD2014, OSI-027, MLN0128, WYE-132, Torin1, PI-103, P7170, PF-04691502, PF-05212384, PKI-587, GNE477, PKI-180, WJD008, XL765, SAR245409, NVP-BEZ235, BGT226, SF1126, GSK2126458, Ku-0063794, WYE-354, NVP-BEZ235, PF-05212384, XL765, Torin 2, WYE-125132, and OSI-027. In some embodiments, the biological sample is selected from the group consisting of whole blood, plasma, serum, saliva, urine, synovial fluid, cerebrospinal fluid, bronchial lavage, ascites fluid, bone marrow aspirate, pleural effusion, tissue, cells, a biopsy, interstitial fluid, and lymphatic fluid. In some embodiments, the protein or peptide comprises a label. In some embodiments, the protein or peptide is attached to a bead, resin, column, or a microplate. In some embodiments, the concentration is determined via an ELISA. In some embodiments, the protein comprises the sequence of SEQ ID NO:2. In some embodiments, the peptide comprises any one of the sequences of SEQ ID NOS:3-35. In some embodiments, the peptide comprises the sequence of SEQ ID NO:3.

In another aspect, provided herein is a diagnostic kit comprising an H1.0K180me2 peptide or H1.0K180me2 protein. In some embodiments, the protein or peptide comprises the sequence selected from the group consisting of SEQ ID NOS: 3-35. In some embodiments, the protein or peptide comprises a label. In some embodiments, the protein or peptide is biotinylated. In some embodiments, the protein or peptide is attached to solid surface. In some embodiments, the protein or peptide is attached a bead, resin, column, or a microplate. In some embodiments, the protein or peptide is provided for the detection of H1.0K180me2 autoantibodies in a sample. In some embodiments, the protein or peptide is provided as a reference standard. In some embodiments, the kit further comprises an H1.0K180me2 antibody. In some embodiments, the kit is used for the detection of Alzheimer's disease, radiation exposure, exposure to a genotoxin, or exposure to a DNA damaging agent. In some embodiments, the kit is used for drug screening. In some embodiments, the kit is used for patient stratification. In some embodiments, the kit is used for treatment selection.

In another aspect, provided herein is a transdermal patch for measuring a concentration of a hypodermal target molecule, comprising: (a) a substrate comprising either (1) an H1.0K180me2 antibody; or (2) an H1.0K180me2 protein or H1.0K180me2 peptide; and (b) a plurality of microneedles. In some embodiments, the substrate comprises an H1.0K180me2 antibody. In some embodiments, the antibody is labeled. In some embodiments, the antibody is an antigen binding fragment. In some embodiments, the substrate comprises an H1.0K180me2 protein or H1.0K180me2 peptide. In some embodiments, the peptide is labeled. In some embodiments, the peptide is biotinylated. In some embodiments, the protein comprises the sequence of SEQ ID NO:2. In some embodiments, the peptide comprises any one of the sequences of SEQ ID NOS:3-35. In some embodiments, the peptide comprises the sequence of SEQ ID NO:3. In some embodiments, the patch is a transdermal microneedle array patch. In some embodiments, the substrate is elastically stretchable.

In another aspect, provided herein is a portable unit for determining whether an individual has been exposed to radiation or a DNA-damaging agent, comprising: (a) a sample collection unit; (b) a reader; (c) an assay module comprising either (1) an H1.0K180me2 antibody; or (2) an H1.0K180me2 protein or H1.0K180me2 peptide; and (d) a plurality of microneedles. In some embodiments, the substrate comprises an H1.0K180me2 antibody. In some embodiments, the antibody is labeled. In some embodiments, the antibody is an antigen binding fragment. In some embodiments, the substrate comprises an H1.0K180me2 protein or H1.0K180me2 peptide. In some embodiments, the protein or peptide comprises a label. In some embodiments, the protein or peptide is biotinylated. In some embodiments, the protein comprises the sequence of SEQ ID NO:2. In some embodiments, the peptide comprises any one of the sequences of SEQ ID NOS:3-35. In some embodiments, the peptide comprises the sequence of SEQ ID NO:3. In some embodiments, the peptide comprises the sequence of SEQ ID NO:2.

In another aspect, provided herein is a test strip suitable for a lateral flow assay of an analyte, comprising a sample receiving zone, wherein the sample receiving zone comprises either (1) an H1.0K180me2 antibody; or (2) an H1.0K180me2 protein or H1.0K180me2 peptide. In some embodiments, the receiving zone comprises an H1.0K180me2 antibody. In some embodiments, the antibody is labeled. In some embodiments, the antibody is an antigen binding fragment. In some embodiments, the receiving zone comprises an H1.0K180me2 protein or H1.0K180me2 peptide. In some embodiments, the protein or peptide comprises a label. In some embodiments, the protein or peptide is biotinylated. In some embodiments, the protein comprises the sequence of SEQ ID NO:2. In some embodiments, the peptide comprises any one of the sequences of SEQ ID NOS:3-35. In some embodiments, the peptide comprises the sequence of SEQ ID NO:3. In some embodiments, the peptide comprises the sequence of SEQ ID NO:2.

In another aspect, provided herein is an in vitro method for dimethylating a histone H1.0 peptide or a histone H1.0 protein comprising contacting the protein or peptide with a methyltransferase enzyme and a methyl donor under conditions that produce a specifically dimethylated protein or peptide, wherein the protein or peptide is specifically dimethylated at a lysine residue corresponding to K180 of a human histone H1.0 protein, and wherein the methyltransferase enzyme is G9A methyltransferase or GLP methyltransferase. In some embodiments, the peptide comprises a sequence selected from the group consisting of SEQ ID NOS:42-74. In some embodiments, the protein comprises the sequence of SEQ ID NO: 1. In some embodiments, the protein or peptide is contacted with a G9A methyltransferase. In some embodiments, the protein or peptide is contacted with a GLP methyltransferase. In some embodiments, the methyl donor is S-Adenosyl-L-Methionine. In some embodiments, the contacting is performed in a methylation buffer. In some embodiments, greater than 50%, greater than 60%, greater than 70%, greater than 80%, greater than 90%, greater than 95%, or even greater than 99% of the product is dimethylated at a lysine residue corresponding to K180 of the human histone H1.0 protein. In a related aspect, provided herein is an antibody that specifically binds the dimethylated protein or peptide produced by the in vitro method provided.

In another aspect, provided herein is a histone H1.0 peptide or a histone H1.0 protein specifically dimethylated at a lysine residue corresponding to K180 of a human histone H1.0 protein produced by a method comprising contacting an H1.0 protein or an H1.0 peptide with a methyltransferase enzyme and methyl donor under conditions that allow for the specific dimethylation, wherein the methyltransferase enzyme is G9A methyltransferase or GLP methyltransferase. In some embodiments, the H1.0 peptide comprises a sequence selected from the group consisting of SEQ ID NOS:42-74. In some embodiments, the H1.0 protein comprises the sequence of SEQ ID NO:1. In some embodiments, the specifically dimethylated H1.0 protein produced comprises the sequence of SEQ ID NO:2. In some embodiments, the specifically dimethylated H1.0 peptide produced comprises the sequence of SEQ ID NOS:3-35. In a related aspect, provided herein is an antibody that specifically binds the dimethylated protein or peptide produced by the in vitro method provided.

In another aspect, provided herein is a kit for the in vitro methylation of an H1.0 protein or H1.0 peptide comprising: (a) an H1.0 protein or an H1.0 peptide; (b) a G9A methyltransferase or GLP methyltransferase enzyme; and a methyl donor. In some embodiments, the kit comprises an H1.0 protein, wherein the H1.0 protein comprises the sequence of SEQ ID NO:1. In some embodiment, the kit comprises an H1.0 peptide. In some embodiments, the H1.0 peptide comprises a sequence selected from the group consisting of SEQ ID NOS:42-74. In some embodiments, the kit comprises a G9A methyltransferase enzyme. In some embodiments, the kit comprises a GLP methyltransferase enzyme. In some embodiments, the methyl donor is S-Adenosyl-L-Methionine. In some embodiments, the kit further comprises a methylation buffer.

In another aspect, provided herein is a complex comprising a histone H1.0 peptide and a methyltransferase enzyme, wherein the complex is in vitro. In some embodiments, the H1.0 peptide comprises a sequence selected from the group consisting of SEQ ID NOs:42-74. In some embodiments, the methyltransferase enzyme is G9A methyltransferase enzyme. In some embodiments, the methyltransferase enzyme is GLP methyltransferase enzyme.

### BRIEF DESCRIPTION OF THE DRAWINGS

FIGS. 1A-1B and show the identification of a histone H1.0 protein comprising a dimethylated lysine at residue 180 (H1.0K180me2) as a post-translational modification upon cellular senescence of hADSCs, using discovery-based mass spectrometry analysis. FIG. 1A shows the mass spectrometry discovery pipeline. FIG. 1B shows the full mass spectra for unmodified (SEQ ID NO: 42) and dimethylated (SEQ ID NO: 3) peptides.

FIGS. 2A-2B show the expression level of six distinct variants of histone H1 (FIG. 2A) and RNA-seq reads (FIG. 2B) from H1.0 in SR (self-renewing) and REP-SEN (replicatively senescent) hADSCs.

FIGS. 3A-D show the specificity of the H1.0K180me2 antibody provided herein. FIG. 3A shows the amino acids flanking histone H1.0K180 (H10K180 (SEQ ID NO: 97); H10K177 (SEQ ID NO: 98); H10K175 (SEQ ID NO: 99); H10K174 (SEQ ID NO: 100); H10K172 (SEQ ID NO: 101); H3K4 (SEQ ID NO: 102); H3K9 (SEQ ID NO: 103); H3K27 (SEQ ID NO: 104); H3K35 (SEQ ID NO: 105) and H3K79 (SEQ ID NO: 106)) as compared with different methylation sites of other lysine residues in the H1.0K180me2.0 protein and the H3.0 protein.

FIG. 3B and FIG. 3C show the slot blot assay results, and demonstrate the specificity of the antibody against H1.0K180me2. FIG. 3D demonstrates the specificity of the antibody against H1.0K180me2. FIG. 3E shows a ClustalW2 alignment of histone H1 variant protein sequences (H1.0 (SEQ ID NO: 1); H1.1 (SEQ ID NO: 108); H1.2 (SEQ ID NO: 109); H1.3 (SEQ ID NO: 110); H1.4 (SEQ ID NO: 111); H1.5 (SEQ ID NO: 112) and H1.X (SEQ ID NO: 113)), depicting the embedment of H1.0K180 in a unique sequence of amino acids not present in other H1 variants.

FIG. 4 shows the use of an indirect ELISA using labeled H1.0K180me2 peptides for the detection of H1.0K180me2 autoantibodies in samples of bodily fluids.

FIG. 5A shows the use of a sandwich ELISA for the direct detection of the H1.0K180me2 antigen, using antibodies against an H1.0K180me2 epitope in samples of bodily fluids. FIG. 5B shows the standard curve for H1.0K180me2 antigen sandwich ELISA test.

FIG. 6 shows that H1.0 mRNA expression increases upon acute DNA damage and genotoxic stress-induced senescence.

FIG. 7A shows the dimethylation of H1.0K180 (H1.0K180me2) on chromatin immediately after DNA damage, by western blot analysis. FIG. 7B shows secretion of H1.0K180me2 from after DNA damage.

FIGS. 8A-8B show that the dimethylation of H1.0K180 occurs upstream of PARP-1 activity. FIG. 8A shows western blot analysis using an H1.0K180me2 antibody, revealing that H1.0K180 methylation occurs upstream of PARP-1 functional activity in the DNA damage repair pathway. FIG. 8B validates inhibition of PARP activity by PARP-1 inhibitor AG14361 to support that H1.0K180 methylation occurs upstream of PARP-1 functional activity in the DNA damage repair pathway.

FIGS. 9A and 9B show LC-MS/MS analysis (FIG. 9A) and slot blot analysis (FIG. 9B) on hADSCs of SR, acute DNA damage, and genotoxic stress induced senescence, revealing that dimethylated H1.0K180 (H1.0K180me2) is released from chromatin upon genotoxic stress induced senescence (FIG. 9A) and is secreted out of the cells into the extracellular matrix/cellular media (FIG. 9B).

FIGS. 10A-10C show the effect of ionizing radiation on H1.0K180me2 levels. FIG. 10A shows that exposure to ionizing radiation induces increased levels of circulating H1.0K180me2 in mouse serum. FIG. 10A and FIG. 10B show a slot blot analysis of the presence (FIG. 10A) and quantification (FIG. 10B) of H1.0K180me2, using antibodies specific for the H1.0K180me2 epitope in the serum. FIG. 10C shows a western blot analysis of presence of H1.0K180me2 epitope levels, using antibodies specific for the H1.0K180me2 epitope, in mouse serum after X-ray irradiation (7 Gy).

FIGS. 11A-11C show age-related accumulation of circulating H1.0K180me2 in the brain tissue and blood serum. Shown are western blot analyses of H1.0K180me2 in mouse (FIG. 11A) and human (FIG. 11B) brain tissue, revealing that its abundance increases with organismal age. FIG. 11C shows H1.0K180me2 levels in human serum samples normalized by total IgG serum levels.

FIGS. 12A-12C demonstrate the utility of measurements of serum H1.0K180me2 as biomarkers of Alzheimer's disease. FIG. 12A shows that serum H1.0K180me2 is decreased in individuals with Alzheimer's disease, when compared to age-matched controls (normalized by serum volume). FIG. 12B shows that serum H1.0K180me2 is decreased in individuals with Alzheimer's disease, when compared to age-matched controls (normalized by total serum IgG levels). FIG. 12C shows that serum H1.0K180me2 is decreased in individuals with Alzheimer's disease, when compared to age-matched controls (normalized by total serum protein concentration).

FIG. 13A shows that human IgG autoantibodies generated to H1.0K180me2 can be detected in different human biofluids (plasma, urine, saliva) using the indirect ELISA method.

FIG. 13B demonstrates the utility of measurements of IgG autoantibodies to H1.0K180me2 as biomarkers of Alzheimer's disease.

FIG. 14A shows a schematic representation of measuring IgM antibodies against H1.0K180me2, under test and normalization conditions.

FIG. 14B shows the results of measurements of total IgMs and a standard curve of anti-IgM molar concentration versus the optical density (O.D.) at 450 nm. The IgM concentration is inferred from the curve. Box plots show total IgM concentrations, demonstrating that total IgM levels do not discriminate between individuals with and without Alzheimer's disease (AD).

FIG. 14C is a schematic representation of the use of an indirect ELISA assay for the measurements and quantification of H1.0K180me2 IgM autoantibodies.

FIG. 14D shows a standard curve of anti-IgG molar concentration versus the optical density (O.D.) at 450 nm. The IgM concentration can be inferred from the curve.

FIG. 14E demonstrates the utility of measurements of IgM autoantibodies directed to H1.0K180me2 as biomarkers of Alzheimer's disease (figures show raw non-normalized data).

FIG. 15A demonstrates the utility of measurements of IgM autoantibodies to H1.0K180me2 as biomarkers of Alzheimer's disease (figures show data normalized to total IgM levels).

FIG. 15B demonstrates the stability and reproducibility of diagnostic characteristics of serological indirect ELISA for the measurements and quantification of H1.0K180me2 IgM autoantibodies between different operators and different laboratory settings.

FIG. 16A demonstrates that correlation between total IgM and H1.0K180me2 IgM autoantibodies is not significant in both Alzheimer's disease and neurological controls patients (based on the R2 value).

FIG. 16B demonstrates that total unmodified H1.0 protein levels (left graph) and total H1.0 IgM autoantibody levels (right graph) do not discriminate between individuals with and without Alzheimer's disease.

FIG. 17 demonstrates that measuring H1.0K180me2 IgG and H1.0K180me2 IgM autoantibodies, and correlating the two measurements, can stratify patients with Alzheimer's disease into distinct sub-populations.

FIG. 18 shows a western blot analysis of H1.0K180me2 in SR hADSCs after bleomycin treatment, revealing that everolimus, a derivative of rapamycin, can act to block H1.0K180me2 appearance upon DNA damage.

FIG. 19 shows a western blot analysis of H1.0K180me2 in SR hADSCs after bleomycin or temozolomide treatment, revealing that temozolomide, a chemotherapy drug that can trigger base excision repair pathways, also leads to methylation of H1.0K180.

FIG. 20 shows the effect of effect of mTOR and PI3K inhibitors on H1.0K180me2 dynamics.

FIGS. 21A-21B show the results of the in vitro G9A methyltransferase methylation assay. The G9A methyltransferase is capable of methylating an H1.0 peptide (FIG. 21A) and is capable of methylating full length H1.0 protein (FIG. 21B).

FIG. 22A shows that in the presence of unmethylated H1.0 peptide, G9A methyltransferase specifically and abundantly dimethylates H1.0K180 (99.9% of all peptides).

FIG. 22B is a tabular representation of efficiency of methylation of G9 and GLP methyltransferase on the unmodified (SEQ ID NO: 42) and dimethylated (SEQ ID NO: 3) peptides.

FIG. 23 shows that in the presence of dimethylated H1.0 peptide, there is minimal further methylation that takes place (only 2.64% of peptides are further methylated).

FIG. 24 shows that in the presence of recombinant, full-length H1.0, G9A methyltransferase methylates C-terminal lysine residues, including H1.0K180me2 (SEQ ID NO: 117).

FIG. 25 shows that in the presence of unmethylated H1.0 peptide, GLP methyltransferase specifically dimethylates H1.0K180 (96.6% of all peptides) to produce H1.0K180me2.

FIG. 26 shows that in the presence of a K180 dimethylated H1.0 peptide, GLP methyltransferase only further methylates H1.0K180 and H1.0K174, and only with a very low efficiency (1.02% of peptides are further methylated).

FIG. 27 shows the methylation of recombinant full-length H1.0 protein with GLP methyltransferase (SEQ ID NO: 118).

FIGS. 28A-28B show the siRNA knockdown of G9A in human adipocyte derived stem cells (hADSCs) led to a reduction (FIG. 28B) in H1.0K180me2 levels upon bleomycin treatment (FIG. 28A).

### DETAILED DESCRIPTION

Provided herein are antibodies that specifically bind a dimethylated antigen, wherein the dimethylated antigen is a histone H1.0 peptide or a histone H1.0 protein comprising a dimethylated lysine residue, wherein the lysine residue corresponds to K180 of a human histone H1.0 protein, and wherein the dimethylated lysine residue is required for binding (H1.0K180me2 antibodies).

Also provided herein are histone H1.0 peptides comprising a dimethylated lysine residue or histone H1.0 proteins comprising a dimethylated lysine residue, wherein the dimethylated lysine residue corresponds to K180 of a human histone H1.0 protein (H1.0K180me2 peptides and H1.0K180me2 proteins).

These H1.0K180me2 antibodies, H1.0K180me2 peptides, and H1.0K180me2 proteins are described herein for therapeutic and diagnostic uses. These H1.0K180me2 antibodies, H1.0K180me2 proteins, and H1.0K180me2 peptides may be used in the treatment of methylated H1.0-related diseases or conditions in an individual. These H1.0K180me2 antibodies, H1.0K180me2 proteins, and H1.0K180me2 peptides may also be used for detecting replicative senescence, DNA damage, genotoxic stress, radiation exposure, Alzheimer's disease, for monitoring therapeutic regimens, patient stratification, drug screening, and may serve as a marker of biological aging in a system.

These and related compositions and methods are described herein.

### I. Dimethylated Proteins and Peptides

### A. Dimethylated H1.0K180me2 Proteins and Peptides

The terms “peptide,” “protein,” and “polypeptide” as used herein to refer to a polymer of amino acids, and unless otherwise specified, include atypical amino acids that function in a similar manner to naturally occurring amino acids.

Provided herein are histone H1.0 proteins and histone H1.0 peptides, comprising a dimethylated lysine residue, wherein the lysine residue corresponds to K180 of a human histone H1.0 protein. A histone H1.0 protein comprising a dimethylated lysine at a residue corresponding to K180 of human H1.0 protein is referred to herein as an “H1.0K180me2 protein.” A histone H1.0 peptide comprising a dimethylated lysine at a residue corresponding to K180 of human H1.0 protein is referred to herein as an “H1.0K180me2 peptide.” They can simply also be referred to collectively as “H1.0K180me2.”

Residue positions in the H1.0 protein discussed herein are identified with respect to a reference amino acid sequence. The human H1.0 histone protein used to identify residue positions is NCBI Reference Sequence: NP_005309.1 and can be accessed at http://www.ncbi.nlm.nih.gov/protein/NP_005309.1. In this case, a reference to “H1.0 residue Lysine 180” or “H1.0K180” or “K180” identifies a residue that, in human histone H1.0, is the 180^{th} amino acid from the N-terminus, wherein the methionine is the first residue. The 180^{th} residue is a lysine (K) in human H1.0. Those of skill in the art appreciate that the K180 residue can have a different position in H1 proteins from different species or in different isoforms.

Use of the term “K180” herein refers to the residue corresponding to K180 of the human H1.0 protein. Similarly, the term “K172”, refers to the residue corresponding to K172 of the human H1.0 protein, and the like.

Table 1 provides the 194-amino acid sequence of the full length human H1.0 protein (NCBI Reference Sequence: NP_005309.1), numbered sequentially from 1-194. The K180 residue is shown as K*.

TABLE 1
Full Length Sequence of Human H1.0 Protein

| | |
|---|---|
| 1 | MTENSTSAPAAKPKRAKASK KSTDHPKYSD MIVAAIQAEK |
| | NRAGSSRQSI QKYIKSHYKV |
| 61 | GENADSQIKL SIKRLVTTGV LKQTKGVGAS GSFRLAKSDE |
| | PKKSVAFKKT KKEIKKVATP |
| 121 | KKASKPKKAA SKAPTKKPKA TPVKKAKKKL AATPKKAKKP |
| | KTVKAKPVKA SKPKKAKPVK* |
| 181 | PKAKSSAKRA GKKK (SEQ ID NO: 1) |

Table 2 provides the 194-amino acid sequence of the full length human H1.0 protein dimethylated at K180, numbered sequentially from 1-194. The K180me2 residue is shown as K(me2).

TABLE 2
Full Length Sequence of Human H1.0
Protein Dimethylated at K180

| | |
|---|---|
| 1 | MTENSTSAPAAKPKRAKASK KSTDHPKYSD MIVAAIQAEK |
| | NRAGSSRQSI QKYIKSHYKV |
| 61 | GENADSQIKL SIKRLVTTGV LKQTKGVGAS GSFRLAKSDE |
| | PKKSVAFKKT KKEIKKVATP |
| 121 | KKASKPKKAA SKAPTKKPKA TPVKKAKKKL AATPKKAKKP |
| | KTVKAKPVKA SKPKKAKPVK(me2) |
| 181 | PKAKSSAKRA GKKK (SEQ ID NO: 2) |

In certain embodiments, the dimethylated H1.0K180me2 peptides (comprising the K180me2 epitope recognized by the H1.0K180me2 antibodies provided herein) comprises the amino acid sequence selected from the sequences presented in Table 3A.

TABLE 3A
Exemplary H1.0K180me2 Peptides

| | |
|---|---|
| AKPVKASKPKKAKPVK(me2)PK | (SEQ ID NO: 3) |
| KPVKASKPKKAKPVK(me2)PK | (SEQ ID NO: 4) |
| PVKASKPKKAKPVK(me2)PK | (SEQ ID NO: 5) |
| VKASKPKKAKPVK(me2)PK | (SEQ ID NO: 6) |
| KASKPKKAKPVK(me2)PK | (SEQ ID NO: 7) |
| ASKPKKAKPVK(me2)PK | (SEQ ID NO: 8) |
| SKPKKAKPVK(me2)PK | (SEQ ID NO: 9) |
| KPKKAKPVK(me2)PK | (SEQ ID NO: 10) |
| PKKAKPVK(me2)PK | (SEQ ID NO: 11) |
| KKAKPVK(me2)PK | (SEQ ID NO: 12) |
| KAKPVK(me2)PK | (SEQ ID NO: 13) |
| AKPVKASKPKKAKPVK(me2)P | (SEQ ID NO: 14) |
| KPVKASKPKKAKPVK(me2)P | (SEQ ID NO: 15) |
| PVKASKPKKAKPVK(me2)P | (SEQ ID NO: 16) |
| VKASKPKKAKPVK(me2)P | (SEQ ID NO: 17) |
| KASKPKKAKPVK(me2)P | (SEQ ID NO: 18) |
| ASKPKKAKPVK(me2)P | (SEQ ID NO: 19) |
| SKPKKAKPVK(me2)P | (SEQ ID NO: 20) |
| KPKKAKPVK(me2)P | (SEQ ID NO: 21) |
| PKKAKPVK(me2)P | (SEQ ID NO: 22) |
| KKAKPVK(me2)P | (SEQ ID NO: 23) |
| KAKPVK(me2)P | (SEQ ID NO: 24) |
| AKPVKASKPKKAKPVK(me2) | (SEQ ID NO: 25) |
| KPVKASKPKKAKPVK(me2) | (SEQ ID NO: 26) |
| PVKASKPKKAKPVK(me2) | (SEQ ID NO: 27) |
| VKASKPKKAKPVK(me2) | (SEQ ID NO: 28) |
| KASKPKKAKPVK(me2) | (SEQ ID NO: 29) |
| ASKPKKAKPVK(me2) | (SEQ ID NO: 30) |
| SKPKKAKPVK(me2) | (SEQ ID NO: 31) |
| KPKKAKPVK(me2) | (SEQ ID NO: 32) |
| PKKAKPVK(me2) | (SEQ ID NO: 33) |
| KKAKPVK(me2) | (SEQ ID NO: 34) |
| KAKPVK(me2) | (SEQ ID NO: 35) |

The H1.0K180me2 proteins and peptides herein may be further conjugated for a variety of purposes including, but not limited to, for use in therapeutics, detection, diagnostics, visualization, quantification, sorting, and for use in biological assays related to their therapeutic use.

In some embodiments, H1.0K180me2 proteins and peptides comprise a label (e.g. conjugated to label), for example a detectable label, a spin label, a colorimetric label, a radioactive label, an enzymatic label, a fluorescent label, or a magnetic label. In an exemplary embodiment, the H1.0K180me2 proteins/peptides are biotinylated. In some embodiments, H1.0K180me2 proteins/peptides are conjugated or attached to a solid surface, for example a bead (e.g. a magnetic, glass or plastic bead), column, resin, or a microplate. In some embodiments, H1.0K180me2 proteins/peptides are coated onto the microplate. In some embodiments, H1.0K180me2 proteins/peptides are conjugated to or comprise an effector molecule including, but not limited to, a radionuclide, a cytotoxin, a chemotherapeutic agent, a drug, a pro-drug, a toxin, an enzyme, an immunomodulator, a pro-apoptotic agent, a cytokine, a hormone, an oligonucleotide, an antisense molecule, a siRNA, and a second antibody.

In some embodiments, the amino acid sequence further comprises an additional terminal residue, for example, for the purpose of conjugation. In some embodiments, the amino acid sequence further comprises a terminal C residue. In such embodiments, the H1.0K180me2 peptide may comprise the amino acid sequence CAKPVKASKPKKAKPVKPK (SEQ ID NO:36), CAKPVKASKPKKAKPVKPKC (SEQ ID NO:37), AKPVKASKPKKAKPVKPKC (SEQ ID NO:38), CAKPVKASKPKKAKPVK^{(me2)}PK (SEQ ID NO:39), CAKPVKASKPKKAKPVK^{(me2)}PKC(SEQ ID NO:40), or AKPVKASKPKKAKPVK^{(me2)}PKC (SEQ ID NO:41). In some embodiments, the H1.0K180me2 peptide comprises a fragment of any one of the H1.0K180me2 peptides provided herein. In some embodiments, the H1.0K180me2 peptide is conjugated to KLH (keyhole limpet hemocyanin), OVA (ovalbumin), BC (bacterial cellulose) or BSA (bovine serum albumin).

The H1.0K180me2 proteins and peptides provided herein are useful both in the detection of pathophysiologies, and for inclusion as a reference standard in methods incorporating a quantitative assessment of H1.0K180me2 presence.

In vitro methylation of protein or peptides traditionally poses challenges of specificity (e.g. specificity of the substrate to be methylated; and control of the same). Described herein are methods that allow for the selective dimethylation of the K180 residue of the H1.0 protein (or peptide fragment thereof), using G9A methyltransferase or GLP methyltransferase.

Provided herein are compositions and methods related to specifically dimethylating a histone H1.0 protein at lysine residue 180 (K180) (H1.0 with a K180me2) and specifically dimethylating a peptide fragment of the histone H1.0 protein at a lysine corresponding to K180.

### B. Production of H1.0K180 Me2 Peptides

The H1.0K180me2 peptides are useful both in the indirect detection of pathophysiologies, and for inclusion as a reference standard for the quantification of the data in methods including, but not limited to (1) molecular diagnostics of at least DNA damage, genotoxic stress (e.g. associated with environmental exposure), radiation exposure, chemotherapy and immunotherapy with an antibody bearing a DNA-damaging payload, radiation therapy, and Alzheimer's disease, (2) monitoring therapeutic regimens and patient stratification, (3) drug screening and (4) therapeutic use.

The H1.0K180me2 peptides provided herein range from 5-193 amino acids (aa) long. In various embodiments, the length of the H1.0K180me2 peptide is 5aa, 6aa, 7aa, 8aa, 9aa, 10aa, 11aa, 12aa, 13aa, 14aa, 15aa, 16aa, 17aa, 18aa, 19aa, 20aa, 21aa, 22aa, 23aa, 24aa, 25aa, 26aa, 27aa, 28aa, 29aa, or 30aa. In certain exemplary embodiments, the length of the H1.0K180me2 peptide is 15aa, 16aa, 17aa, 18aa, 19aa, or 20aa.

Listed in Table 3A are exemplary H1.0K180me2 peptides that can be synthetically produced using the methods and compositions described herein. In some embodiments, the H1.0K180me2 peptides of the invention comprise one of the sequences selected from those presented in Table 3A. In some embodiments, the H1.0K180me2 peptide consists one of the sequences selected from those presented in Table 3A. In an exemplary embodiment, the H1.0K180me2 peptide comprises the sequence of SEQ ID NO:3, SEQ ID NO:4, or SEQ ID NO: 5. In an exemplary embodiment, the H1.0K180me2 peptide consists of the sequence of SEQ ID NO:3, SEQ ID NO:4, or SEQ ID NO:5. The peptides provided in Table 3A can further comprise a label, e.g. biotin.

Provided herein are methods for the in vitro production of H1.0K180me2 peptides (synthetically produced H1.0K180me2 peptides). Generally the method comprises contacting a substrate peptide with a G9A methyltransferase enzyme or a G9A-like protein (GLP) methyltransferase enzyme and a methyl donor under conditions that allow for the specific dimethylation of a lysine residue corresponding to K180 of the histone H1.0 protein (in vitro methylation). In various embodiments, the substrate H1.0 peptide comprises a sequence selected from the group of sequences presented in Table 3B. In related embodiments, the peptide is a sequence selected from the group of sequences presented in Table 3B.

The peptides used for methylation may be synthetically produced or produced using methods familiar to those with skill in the art.

TABLE 3B
H1.0 Peptides for In vitro Methylation

| | |
|---|---|
| AKPVKASKPKKAKPVKPK | (SEQ ID NO: 42) |
| KPVKASKPKKAKPVKPK | (SEQ ID NO: 43) |
| PVKASKPKKAKPVKPK | (SEQ ID NO: 44) |
| VKASKPKKAKPVKPK | (SEQ ID NO: 45) |
| KASKPKKAKPVKPK | (SEQ ID NO: 46) |
| ASKPKKAKPVKPK | (SEQ ID NO: 47) |
| SKPKKAKPVKPK | (SEQ ID NO: 48) |
| KPKKAKPVKPK | (SEQ ID NO: 49) |
| PKKAKPVKPK | (SEQ ID NO: 50) |
| KKAKPVKPK | (SEQ ID NO: 51) |
| KAKPVKPK | (SEQ ID NO: 52) |
| AKPVKASKPKKAKPVKP | (SEQ ID NO: 53) |
| KPVKASKPKKAKPVKP | (SEQ ID NO: 54) |
| PVKASKPKKAKPVKP | (SEQ ID NO: 55) |
| VKASKPKKAKPVKP | (SEQ ID NO: 56) |
| KASKPKKAKPVKP | (SEQ ID NO: 57) |
| ASKPKKAKPVKP | (SEQ ID NO: 58) |
| SKPKKAKPVKP | (SEQ ID NO: 59) |
| KPKKAKPVKP | (SEQ ID NO: 60) |
| PKKAKPVKP | (SEQ ID NO: 61) |
| KKAKPVKP | (SEQ ID NO: 62) |
| KAKPVKP | (SEQ ID NO: 63) |
| AKPVKASKPKKAKPVK | (SEQ ID NO: 64) |
| KPVKASKPKKAKPVK | (SEQ ID NO: 65) |
| PVKASKPKKAKPVK | (SEQ ID NO: 66) |
| VKASKPKKAKPVK | (SEQ ID NO: 67) |
| KASKPKKAKPVK | (SEQ ID NO: 68) |
| ASKPKKAKPVK | (SEQ ID NO: 69) |
| SKPKKAKPVK | (SEQ ID NO: 70) |
| KPKKAKPVK | (SEQ ID NO: 71) |
| PKKAKPVK | (SEQ ID NO: 72) |
| KKAKPVK | (SEQ ID NO: 73) |
| KAKPVK | (SEQ ID NO: 74) |

Generally, the method for dimethylating a histone H1.0 peptide comprises contacting the peptide with a methyltransferase enzyme and a methyl donor under conditions that produce a specifically dimethylated peptide, wherein the peptide is specifically dimethylated at a lysine residue corresponding to K180 of a human histone H1.0 protein, and wherein the methyltransferase enzyme is G9A methyltransferase or GLP methyltransferase.

In embodiments of the methods for in vitro production of H1.0K180me2 peptide, the G9A methyltransferase may be recombinant G9A methyltransferase, a purified G9A mammalian methyltransferase, a human G9A methyltransferase, a mouse G9A methyltransferase, or the like. The G9A methyltransferase includes enzymatically active orthologs, chimeras and artificially or naturally produced isoforms containing enzymatic domains.

In embodiments of the methods for in vitro production of H1.0K180me2 peptide, the GLP methyltransferase may be recombinant GLP methyltransferase, a purified GLP mammalian methyltransferase, a human GLP methyltransferase, a mouse GLP methyltransferase, or the like. The GLP methyltransferase includes enzymatically active orthologs, chimeras and artificially or naturally produced isoforms containing enzymatic domains.

In embodiments of the methods for the in vitro production of H1.0K180me2 peptide, the methyl donor may be S-Adenosyl-L-Methionine (SAM).

In embodiments of the methods for in vitro production of H1.0K180me2 peptide, the contacting may be done in a methylation buffer.

In embodiments of the methods for the in vitro production of H1.0K180me2 peptide, the peptide may comprise a label (e.g. biotin) prior to the methylation.

In embodiments of the methods for the in vitro production of H1.0K180me2 peptide, the peptide may be conjugated with a label (e.g. biotin) after the methylation.

In embodiments of the methods for the in vitro methylation of the H1.0K180me2 peptide substrate, greater than 50%, 60%, 65%, 70%, 75%, 80%, 85%, 90%, 95%, 99%, or even 99.9% of the product comprises K180me2. Likewise, in embodiments of the methods for the in vitro production of H1.0K180me2 peptide, in various embodiments, less than 50%, 40%, 30%, 25%, 20%, 15%, 10%, 5%, 4%, 3%, 2%, 1%, 0.5%, or less than 0.35% of the product also contains other methylated residues (e.g. K172me1, K172me2, K172me3, K174me1, K174me2, K174me3, K175me1, K175me2, K175me3, K177me1, K177me2, K177me3, K166me1, K166me2, K166me3, K180me1, and/or K180me3). In exemplary embodiments, less than 0.35% of the product having the K180me2 also comprises K174me3, K175me3, and K177me1; less than 1.25% of the product having the K180me2 also comprises K166me1; and less than 1.04% of the product having the K180me2 comprises K174me1 and K180me3.

In some embodiments an H1.0K180me2 peptide binds an antibody that is specific for an H1.0K180me2 antigen (an H1.0K180me2 antibody). Provided herein, is an antibody that specifically binds a dimethylated antigen, wherein the dimethylated antigen is a histone H1.0 peptide comprising a dimethylated lysine residue, wherein the lysine residue corresponds to K180 of a human histone H1.0 protein.

In certain embodiments, an H1.0K180me2 peptide binds an H1.0K180me2 antibody with a dissociation constant (Kd) of about 0.0001 nM to about 1 μM. For example, Kd of the peptide may be about 1 μM, about 100 nM, about 50 nM, about 10 nM, about 5 nM, about 1 nM, about 0.5 nM, about 0.1 nM, about 0.05 nM, about 0.01 nM, about 0.005 nM, about 0.001 nM, about 0.0005 nM, or even about 0.0001 nM.

In some embodiments, an H1.0K180me2 peptide is specific for the anti-human H1.0K180me2 antibody. In some embodiments, an H1.0K180me2 peptide is cross reactive with an H1.0K180me2 antibody from other species.

In some embodiments, an H1.0K180me2 peptide is selective for the H1.0K180me2 antibody and exhibits little or no binding to H1.0K180me1 or H1.0K180me3 antibodies.

In some embodiments, the binding preference of an H1.0K180me2 peptide (e.g., affinity) for the H1.0K180me2 antibody is generally at least about 2-fold, about 5-fold, or at least about 10-, 20-, 50-, 10²-, 10³-, 10⁴, 10⁵, or 10⁶-fold over a non-specific target antibody (e.g. a randomly generated antibody).

The H1.0K180me2 peptides can be equally specific/selective for antibodies carrying a payload (e.g. including but not limited to a radionuclide, a cytotoxin, a chemotherapeutic agent, a drug, a pro-drug, a toxin, an enzyme, an immunomodulator, a pro-apoptotic agent, a cytokine, a hormone, an antagonist, an agonist or a receptor decoy).

The H1.0K180me2 peptides provided herein may be further conjugated for a variety of purposes including, but not limited to, for use in detection, diagnostics, visualization, quantification, sorting, therapeutics, and for use in biological assays.

In some embodiments, an H1.0 peptide or the H1.0K180me2 peptide is comprises a label (e.g. conjugated either before or after the methylation), for example a detectable label, a spin label, a colorimetric label, a radioactive label, an enzymatic label, a fluorescent label, a magnetic label, or the like.

In some embodiments, provided herein is a complex comprising a histone H1.0 peptide and a methyltransferase enzyme, wherein the complex is in vitro (e.g. in a test tube, tube, reaction chamber, reaction vessel, etc.). In some embodiments, the H1.0 peptide comprises a sequence selected from the group consisting of SEQ ID NOs:42-74. In some embodiments, the methyltransferase enzyme is G9A methyltransferase enzyme. In some embodiments, the methyltransferase enzyme is GLP methyltransferase enzyme.

### C. Production of H1.0K180 Me2 Proteins

The full length H1.0K180me2 proteins described herein are useful both in the detection of pathophysiologies, and for inclusion as a reference standard in methods including, but not limited to (1) molecular diagnostics of at least DNA damage, genotoxic stress (e.g. associated with environmental exposure), radiation exposure, chemotherapy and immunotherapy with an antibody bearing a DNA-damaging payload, radiation therapy, and Alzheimer's disease, (2) monitoring therapeutic regimens and patient stratification, (3) drug screening, and (4) used as therapeutics.

In some embodiments, provided herein are in vitro methods for dimethylating a histone H1.0 protein comprising contacting the protein with a methyltransferase enzyme and a methyl donor under conditions that produce a specifically dimethylated protein, wherein the protein is specifically dimethylated at a lysine residue corresponding to K180 of a human histone H1.0 protein, and wherein the methyltransferase enzyme is a G9A methyltransferase or GLP methyltransferase.

In some embodiments, provided herein are methods for the in vitro production of an H1.0K180me2 protein. In one embodiment, the method comprises contacting a full length H1.0 protein with a G9A methyltransferase enzyme and a methyl donor under conditions that allow for the specific dimethylation the K180 residue. The G9A methyltransferase includes enzymatically active orthologs, chimeras and artificially or naturally produced isoforms containing enzymatic domains.

In some embodiments, provided herein are methods for the in vitro production of an H1.0K180me2 protein. In one embodiment, the method comprises contacting a full length H1.0 protein with a GLP methyltransferase enzyme and a methyl donor under conditions that allow for the specific dimethylation the K180 residue. The GLP methyltransferase includes enzymatically active orthologs, chimeras and artificially or naturally produced isoforms containing enzymatic domains.

The full length H1.0 unmethylated protein substrate used for the in vitro methylation may be isolated, synthetically produced, or recombinantly produced, using methods familiar to those with skill in the art. Provided herein are nucleic acids encoding the H1.0K180me2 protein. Also provided herein are vectors comprising any of the nucleic acids encoding for the H1.0K180me2 proteins provided herein.

In some embodiments of the methods for the in vitro methylation or in vitro production of an H1.0K180me2 protein, the G9A methyltransferase may be a recombinant G9A methyltransferase, a purified G9A mammalian methyltransferase, a human G9A methyltransferase, a mouse G9A methyltransferase, or the like.

In some embodiments of the methods for the in vitro methylation or in vitro production of H1.0K180me2 protein, the GLP methyltransferase may be a recombinant GLP methyltransferase, a purified GLP mammalian methyltransferase, a human GLP methyltransferase, or a mouse GLP methyltransferase.

In some embodiments of the methods for the in vitro production of H1.0K180me2 protein, the protein comprises may comprise label (e.g. biotin) prior to the methylation.

In some embodiments of the methods for the in vitro production of H1.0K180me2 protein, the protein may be conjugated with a label (e.g. biotin) after the methylation.

In some embodiments of the methods for the in vitro methylation or in vitro production of H1.0K180me2 protein, the methyl donor is S-Adenosyl-L-Methionine.

In some embodiments of the methods for the in vitro production of H1.0K180me2 protein, the contacting is done in a methylation buffer.

In embodiments of the methods for the in vitro methylation of the H1.0K180me2 protein substrate, greater than 50%, 60%, 65%, 70%, 75%, 80%, 85%, 90%, 95%, 96%, 97%, 98%, 99%, or even 99.9% of the product may comprise K180me2. Likewise, in some embodiments of the methods for the in vitro production of H1.0K180me2 protein, less than 50%, 40%, 30%, 25%, 20%, 15%, 10%, 5%, 4%, 3%, 2%, 1%, 0.5%, or less than 0.35% of the product also contains other methylated residues (e.g. K172me1, K172me2, K172me3, K174me1, K174me2, K174me3, K175me1, K175me2, K175me3, K177me1, K177me2, K177me3, K166me1, K166me2, K166me3, K180me1, and/or K180me3). In exemplary embodiments, less than 0.35% of the product having the K180me2 also comprises K174me3, K175me3, and K177me1; less than 1.25% of the product having the K180me2 also comprises K166me1; and/or less than 1.04% of the product having the K180me2 comprises K174me1 and K180me3.

Provided herein is an antibody that specifically binds a dimethylated antigen, wherein the dimethylated antigen is found in a histone H1.0 protein comprising a dimethylated lysine residue, wherein the lysine residue corresponds to K180 of a human histone H1.0 protein.

In some embodiments the H1.0K180me2 protein is selective for an antibody that is specific for H1.0K180me2 (H1.0K180me2 antibody). In some embodiments, the peptide binds an antibody that is non-specific for H1.0K180me2.

In certain embodiments, the H1.0K180me2 proteins bind an H1.0K180me2 antibody with a dissociation constant (Kd) of about 0.0001 nM to about 1 μM. For example, the Kd may be about 1 μM, about 100 nM, about 50 nM, about 10 nM, about 5 nM, about 1 nM, about 0.5 nM, about 0.1 nM, about 0.05 nM, about 0.01 nM, about 0.005 nM, about 0.001 nM, about 0.0005 nM, or even about 0.0001 nM.

In some embodiments, the H1.0K180me2 protein is specific for the anti-human H1.0K180me2 antibody. In some embodiments, an H1.0K180me2 protein is cross reactive with an H1.0K180me2 antibody from other species.

In some embodiments, the H1.0K180me2 protein is selective for the H1.0K180me2 antibody and exhibits little or no binding to H1.0K180me1 or H1.0K180me3 antibodies.

In some embodiments, the binding preference of the H1.0K180me2 protein (e.g., affinity) for the H1.0K180me2 antibody is generally at least about 2-fold, about 5-fold, or at least about 10-, 20-, 50-, 10²-, 10³-, 10⁴, 10⁵, or 10⁶-fold over a non-specific target antibody (e.g. a randomly generated antibody).

The H1.0K180me2 proteins provided herein may be further conjugated for a variety of purposes including, but not limited to, for use in detection, diagnostics, visualization, quantification, sorting, therapeutics, and for use in biological assays.

In some embodiments, the H1.0 protein (H1.0 substrate) or the H1.0K180me2 proteins are conjugated to a label (either before or after methylation), for example a detectable label, a spin label, a colorimetric label, a radioactive label, an enzymatic label, a fluorescent label, a magnetic label, or the like.

II. Antibodies that Bind to Dimethylated Histone H1.0 Proteins and Peptides

### A. H1.0K180 Me2 Antibodies

Provided herein are antibodies that specifically bind a dimethylated antigen, wherein the dimethylated antigen is a histone H1.0 peptide or histone H1.0 protein comprising a dimethylated lysine residue, wherein the lysine residue corresponds to K180 of a human histone H1.0 protein. This dimethylated antigen is the H1.0K180me2 antigen, also referred to as the H1.0K180me2 epitope. These antibodies specifically bind the H1.0K180me2 epitope on H1.0K180me2 proteins and H1.0K180me2 peptides. These antibodies specifically bind the dimethylated antigen (specifically bind to an H1.0K180me2 epitope) and require the presence of the dimethyl group at K180 for binding. The terms “anti-H1.0K180me2” or “H1.0K180me2 antibody” or “anti-H1.0K180me2 antibody” interchangeably refer to these antibodies.

The term “antibody” as used herein throughout is in the broadest sense and includes, but is not limited to, a monoclonal antibody, polyclonal antibody, human antibody, humanized antibody, non-human antibody, chimeric antibody, bispecific antibody, multi-specific antibody, polyfunctional antigen-binding fragment (e.g Fab fragment, a Fab′2 fragment, a CDR or a ScFv), antibody-drug conjugates, and other antibody fragments that retain specificity for an H1.0K180me2 antigen. In some embodiments, the antibody is a single chain antibody that retains the specificity for an H1.0K180me2 antigen.

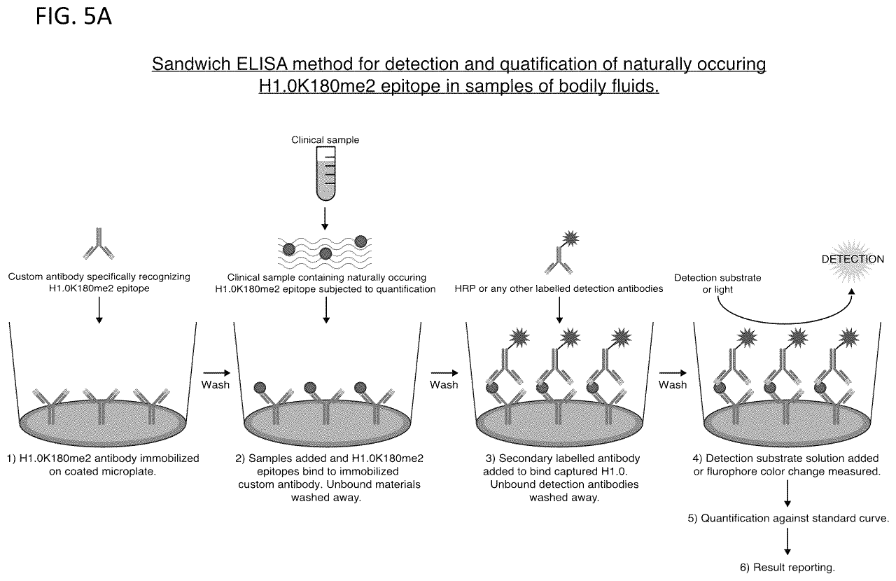

Schematic binding of the H1.0K180me2 antibody is shown in FIG. 5A.

In some embodiments, the H1.0K180me2 antibodies provided herein are diagnostic antibodies.

In some embodiments, the H1.0K180me2 antibodies provided herein are therapeutic antibodies.

In some embodiments, the H1.0K180me2 antibody exists in a high titer.

In some embodiments, the H1.0K180me2 antibody is an affinity purified antibody.

The H1.0K180me2 antibodies provided herein may be further conjugated for a variety of purposes including, but not limited to, for use in detection, diagnostics, visualization, quantification, sorting, therapeutics, and for use in biological assays.

In some embodiments, the H1.0K180me2 antibodies comprise a label (e.g. are conjugated to a label), for example a detectable label, a spin label, a colorimetric label, a radioactive label, an enzymatic label, a fluorescent label, or a magnetic label.

In some embodiments, an antibody is conjugated or attached to a solid surface, for example a bead (e.g. a magnetic, glass or plastic bead), column, resin or a microplate. In some embodiments, an antibody is coated onto the microplate.

In some embodiments, an antibody is conjugated to or comprise an effector molecule including, but not limited to, a radionuclide, a cytotoxin, a chemotherapeutic agent, a drug, a pro-drug, a toxin, an enzyme, an immunomodulator, a pro-apoptotic agent, a cytokine, a hormone, an oligonucleotide, an antisense molecule, a siRNA, and a second antibody.

It is appreciated that H1.0K180me2 biomarker may be chromatin bound, or may be released from the chromatin into the nucleus, cytoplasm, or the extracellular space. The antibodies provided herein can bind extracellular H1.0K180me2 and/or intracellular H1.0K180me2. If intracellular, the H1.0K180me2 antigen may be further bound to chromatin, or released in the nucleus, released from the nucleus into the cytoplasmic space, or further localized in a cytoplasmic sub-structure.

In some embodiments, the antibody (e.g. a therapeutic antibody) is a neutralizing antibody, and the antibody neutralizes one or more biological activities of H1.0K180me2. For example, the antibody may bind extracellular H1.0K180me2 and neutralize any binding or signaling activity it may possess.

The antibodies provided herein may be of any immunoglobulin type such as IgG, IgA, IgE, IgD, or IgM. In some embodiments, the antibody is of the IgG subtype and may be an IgG1 antibody, an IgG2 antibody, an IgG3 antibody, or an IgG4 antibody.

Provided herein are antibodies specific for H1.0K180me2 from any species. In some embodiments, the H1.0K180me2 antibody is specific for human H1.0K180me2. In some embodiments, the H1.0K180me2 antibody is cross reactive with H1.0K180me2 from other species.

The antibodies provided herein bind H1.0K180me2 with specificity. In some embodiments, these antibodies bind H1.0K180me2 with specificity and selectivity.

The antibodies provided herein specifically bind a dimethylated antigen, wherein the dimethylated H1.0 antigen comprises a dimethylated lysine residue, wherein the lysine residue corresponds to K180 of a human histone H1.0 protein (H1.0K180me2 antigen)—in some embodiments, the dimethylated antigen does not comprise any other lysine residues that are methylated.

In some embodiments, the H1.0K180me2 antibody does not bind, or only minimally binds if antigen comprises a dimethylated lysine residue, wherein the lysine residue corresponds to K166, K172, K174, K175, and/or K177 of a human histone H1.0 protein.

In some embodiments, the H1.0K180me2 antibody does not bind, or only minimally binds, an antigen that comprises one or more of the following residues: K172me1, K172me2, K172me3, K174me1, K174me2, K174me3, K175me1, K175me2, K175me3, K177me1, K177me2, K177me3, K166me1, K166me2, K166me3, K180me1, and/or K180me3.

In some embodiments, the H1.0K180me2 antibody does not bind, or only minimally binds, an antigen that comprises a dimethylated K180 residue, but also comprises one or more of the following residues: K172me1, K172me2, K172me3, K174me1, K174me2, K174me3, K175me1, K175me2, K175me3, K177me1, K177me2, K177me3, K166me1, K166me2, K166me3, K180me1, and/or K180me3.

In some embodiments, the H1.0K180me2 antibody does not bind, or only minimally binds if the antigen comprises a monomethylated lysine residue at lysine residues corresponding to K166, K172, K174, K175, K177, and/or K180 of a human histone H1.0 protein.

In some embodiments, the H1.0K180me2 antibody does not bind, or only minimally binds if the antigen comprises a trimethylated lysine residue at lysine residues corresponding to K166, K172, K174, K175, K177, and/or K180 of a human histone H1.0 protein.

In some embodiments, the H1.0K180me2 antibody is selective for dimethylation at residue K180, and exhibits little or no binding of an H1.0K180me1 epitope, or an H1.0K180me3 epitope.

The H1.0K180me2 antibody binds the H1.0K180me2 epitope in any medium.

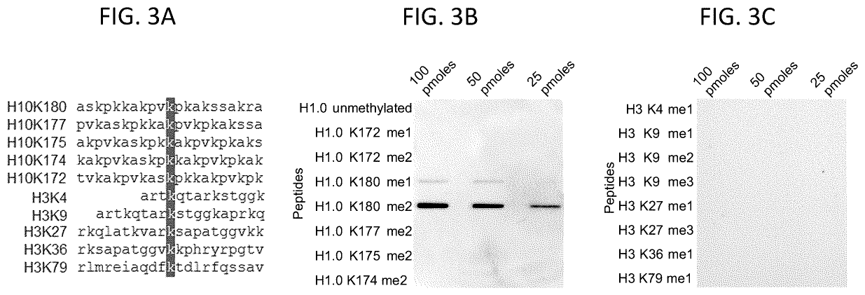

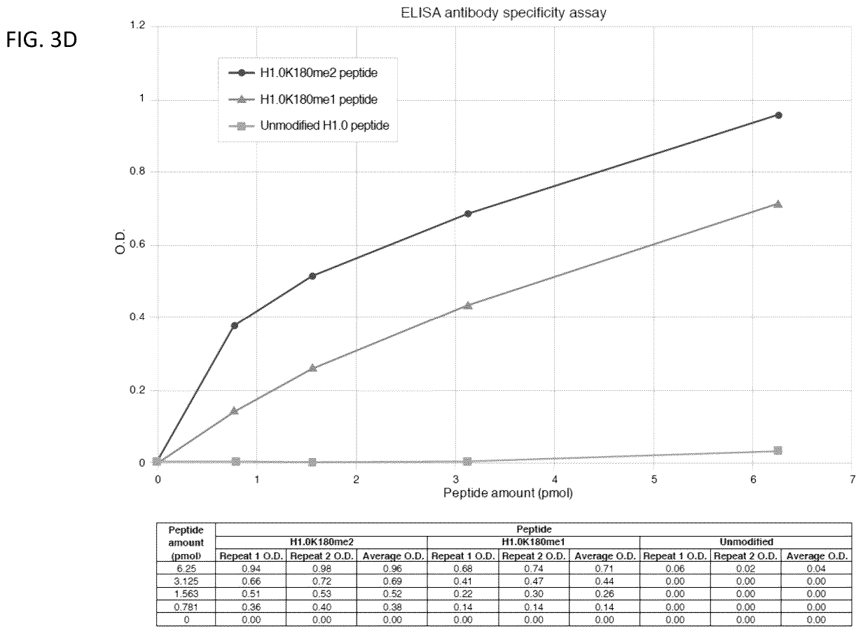

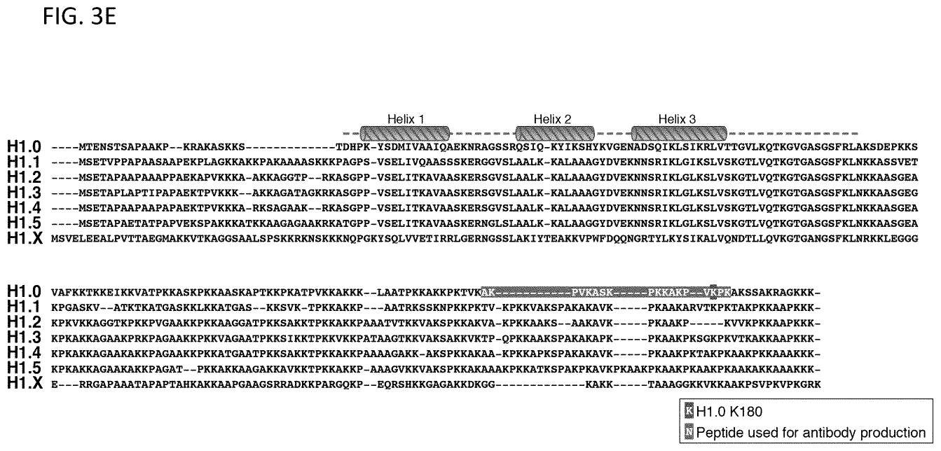

In some embodiments, the H1.0K180me2 antibody displays at least 1.5-fold, 2-fold, 2.5-fold, 2.7-fold, 5-fold, or even 10-fold more specificity (binding preference, affinity) for the dimethylated antigen at K180 (H1.0K180me2 antigen), than a monomethylated antigen at K180 (H1.0K180me1 antigen). (FIG. 3D) In some embodiments the specificity for an H1.0K180me2 antigen is generally at least about 2-fold, about 5-fold, or at least about 10-, 20-, 50-, 10²-, 10³-, 10⁴, 10⁵, or 10⁶-fold over a non-specific target molecule (e.g. a randomly generated molecule lacking the specifically recognized site(s)), over a monomethylated K180 residue, over a trimethylated K180 residue, or over an H1.0 protein methylated at any other residue.

In some embodiments, the binding efficiency of the H1.0K180me2 antibody is monitored by an ELISA assay. In some embodiments, the antibody is at least 2.7-fold more efficient at binding an H1.0K180me2 peptide than an H1.0K180me1 peptide. In some embodiments, 1 molecule of the antibody recognizes 1 out of 117 molecules of an H1.0K180me2 peptide, but only 1 out of 316 molecules of an H1.0K180me1 peptide.

In certain embodiments, an antibody provided herein has a dissociation constant (Kd) of range of 0.0001 nM to 1 μM. For example, Kd of the antibody may be about 1 μM, about 100 nM, about 50 nM, about 10 nM, about 5 nM, about 1 nM, about 0.5 nM, about 0.1 nM, about 0.05 nM, about 0.01 nM, about 0.005 nM, about 0.001 nM, about 0.0005 nM, or even about 0.0001 nM.

### B. Generation of H1.0K180 Me2 Antibodies

A variety of immunoassay formats may be used to select antibodies specifically immunoreactive with H1.0K180me2. For example, solid-phase ELISA immunoassays may be used to select monoclonal antibodies specific to H1.0K180me2 (see, e.g., Harlow and Lane (1988) Antibodies, A Laboratory Manual, Cold Spring Harbor Publications, New York, for a description of immunoassay formats and conditions that may be used to determine specific immunoreactivity).

Production of the antibodies provided herein may be by any method known to those with skill in the art. For example, in some embodiments, the antibodies are produced by recombinant cells engineered to express the desired VH, VL and constant domains of the desired antibody. In some embodiments the antibodies are produced by hybridomas.

In some embodiments, any peptide comprising the H1.0K180me2 antigen, optionally linked to the immunogenic carrier, is used for immunization using standard protocols. In some embodiments, a peptide comprising a sequence from those presented in Table 3A, optionally linked to the immunogenic carrier, is used for immunization using standard protocols. In an exemplary embodiment, a peptide comprising AKPVKASKPKKAKPVK^{me2}PK (SEQ ID NO:3), optionally linked to the immunogenic carrier, and is used for immunization using standard protocols. The quality and titer of generated antibodies may be assessed using techniques known to those in the art.

The inventive compositions described herein also include nucleic acids encoding the antibodies, vectors comprising any of the nucleic acids encoding the antibodies, and host cells comprising any such vectors.

As those of skill in the art readily appreciate, antibodies can also be prepared by any of a number of commercial services.

### III. Diagnostics

### A. Direct and Indirect Detection of H1.0K180 Me2

The H1.0K180me2 antibodies, H1.0K180me2 proteins, and H1.0K180me2 peptides provided herein are useful for a variety of diagnostic purposes.

Provided herein are assays for both the direct and indirect detection and quantification of the concentration of H1.0K180me2. Such quantification may be useful for detecting replicative senescence, DNA damage, genotoxic stress, radiation exposure, Alzheimer's disease, and biological aging. The quantification may also useful for monitoring therapeutic regimens, drug screening, and stratification of patients as responders or non-responders to drug treatments aimed to restore cell viability, prevent DNA damage, increase cellular metabolism and autophagy, inhibit cellular senescence and block insoluble protein waste accumulation in cellular cytoplasm. Depending on the application, H1.0K180me2 may be detected and quantified in vivo, in vitro, ex vivo, in situ, or in a cell-free system.

Direct detection of H1.0K180me2 involves detecting H1.0K180me2 using an H1.0K180me2 antibody.

Indirect detection of H1.0K180me2 involves using H1.0K180me2 proteins or H1.0K180me2 peptides, which bind autoantibodies generated in response to presence of the H1.0K180me2 antigen. In this context the H1.0K180me2 protein can be referred to as an H1.0K180me2 autoantibody-binding protein and the H1.0K180me2 peptide as an H1.0K180me2 autoantibody-binding peptide.

H1.0K180me2 may be detected by any of number of methods well known to those of skill in the art. The H1.0K180me2 antibodies, H1.0K180me2 proteins and H1.0K180me2 peptides provided herein are readily used in a variety of immunoassays. These immunoassays include, but are not limited to enzyme-linked immunosorbent assay (ELISA), Western blot, radioimmunoassay (RIA), flow cytometry, lateral flow immunoassay, slot blot, magnetic immunoassay, a radioimmunoassay, indirect immunofluorescence assay, direct immunofluorescence assay, surround Optical Fiber Immunoassay (SOFIA), spectrophotometry, radiography, electrophoresis, immunoelectrophoresis, capillary electrophoresis, high performance liquid chromatography (HPLC), thin layer chromatography (TLC), hyperdiffusion chromatography, fluid or gel precipitation reactions, immunodiffusion, spectrometry, mass spectrometry, quantitative mass-spectrometry, any type of multiplex assay, and any type of microfluidic assay.

The H1.0K180me2 antibodies and H1.0K180me2 proteins or peptides provided herein may comprise a label (e.g. conjugated to a label) for example a detectable label, a spin label, a colorimetric label, a radioactive label, an enzymatic label, a fluorescent label, or a magnetic label.

The H1.0K180me2 antibodies and H1.0K180me2 proteins and peptides provided herein may be comprise a detectable label. The detectable group may be any material having a detectable physical or chemical property, for example detectable by spectroscopic, photochemical, biochemical, immunochemical, fluorescent, electrical, optical or chemical methods. Useful labels in the present invention include, but are not limited to, magnetic beads (e.g. DYNABEADS®), fluorescent dyes (e.g., fluorescein isothiocyanate, red, rhodamine, and the like), radiolabels (e.g., ³H, ¹²⁵I, ³⁵S, ¹⁴C, or ³²P), enzymes (e.g., LacZ, CAT, horseradish peroxidase, alkaline phosphatase and others, commonly used as detectable enzymes, either as marker gene products or in an ELISA), biotin, avidin, or streptavidin and colorimetric labels such as colloidal gold colored glass or plastic (e.g. polystyrene, polypropylene, latex, etc.) beads, and nanoparticles. In an exemplary embodiment, biotin is the label.

The labels provided herein may be coupled directly or indirectly to the desired component of the assay according to methods well known in the art. As indicated above, a wide variety of labels may be used, with the choice of label depending on the sensitivity required, ease of conjugation of the compound, stability requirements, available instrumentation, and disposal provisions. Labels are often attached by indirect methods. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands may be used. Where a ligand has a natural anti-ligand, for example, biotin, it may be used in conjunction with the labeled, naturally occurring anti-ligands. Alternatively, any haptenic or antigenic compound may be used in combination with an antibody. Components can also be conjugated directly to signal-generating compounds, e.g., by conjugation with an enzyme or fluorophore. Enzymes of interest as labels include, but are not limited to, hydrolases, phosphatases, esterases, glycosidases, or oxitranscription factoreductases, and peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol.

Methods of detecting labels are well known to those of skill in the art. Thus, for example, where the label is a radioactive label, methods for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels may be detected by providing appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

In an immunoassay detecting autoantibodies specific to H1.0K180me2, specific secondary antibodies can be utilized to differentiate between IgG, IgM, IgA, IgE, and IgD autoantibody types.

Upon detection, the concentration of H1.0K180me2 in a particular fraction may be quantified, for example quantification of H1.0K180me2 in an intra-cellular fraction, in a soluble and chromatin bound fraction, or in a cytoplasmic fraction. For example, an increase in the concentration of H1.0K180me2 in a cytoplasm fraction is indicative of a senescent state of the cells. In some embodiments intact cells, cells in culture, or cells in slice culture are imaged to visualize the localization of an H1.0K180me2. For example, an increase in a non-nuclear sub-localization of H1.0K180me2 is indicative of a senescent state. In some embodiments release of H1.0K180me2 into the cytosol or extracellular matrix is indicative of a senescent state.

Detection may be carried out on any biological sample. Biological samples include, but are not limited to whole blood, plasma, serum, saliva, urine, feces, synovial fluid, cerebrospinal fluid, bronchial lavage, ascites fluid, bone marrow aspirate, pleural effusion, tissue, cells, a biopsy, interstitial fluid, lymphatic fluid, or fractions thereof derived from the individual. In some embodiments, the biological sample comprises cells and the cells are in culture, in a suspension, on a slide, in intact tissue, or in preparation ready for a FACs analysis.

Biological samples are obtained from individuals. As used herein, an individual refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Individuals may be male or female. In one embodiment, the individual is a female. In one embodiment, the individual is a male.

In some embodiments the individual is greater than 50 years old. In some embodiments of the method described herein, the individual is less than 50 years old. In some embodiment, of the method described herein, the individual is at least 50 years old, is at least 55 years old, is at least 60 years old, is at least 65 years old, is at least 70 years old, is at least 75 years old, or is at least 80 years old. In an exemplary embodiment, the individual is at least 60 years old.

Biological samples are obtained according to standard methods well known to those of skill in the art. The sample is optionally pretreated as necessary by dilution in an appropriate buffer solution or concentrated, if desired. Any of a number of standard aqueous buffer solutions, employing one of a variety of buffers, such as phosphate, Tris, or the like, at physiological pH may be used.

The direct detection methods described herein may be used to quantify the concentration of H1.0K180me2 in the biological sample. In some embodiments, an H1.0K180me2 protein or peptide may be used in tandem, for example, as a positive control or as competitor in a competitive immunoassay, and may be labeled or not, depending on the format of the assay to be carried out.

The indirect detection methods described herein may also be used to quantify the concentration of H1.0K180me2 autoantibodies in the biological sample. In some embodiments, an H1.0K180me2 antibody may be used in tandem, for example, as a positive control or as competitor in a competitive immunoassay, and may be labeled or not, depending on the format of the assay to be carried out.

One of skill will appreciate that in some embodiments, it may be necessary to compare the determined concentrations of the H1.0K180me2 antigen or autoantibodies to a control (i.e. reference control). The relative comparison may allow, for example, for the determination of whether an individual has, or is at risk of developing a disease (e.g. Alzheimer's disease) or whether the individual is responsive, or may be responsive to a particular treatment (e.g. an Alzheimer's disease treatment; or a treatment with a rapalogue). Controls may be age-matched controls; sex-matched controls; age- and sex-matched controls; healthy controls; unmanipulated controls; or a reference standard representing a compilation of reference standards. The comparisons can also be made to a sample from the same individual prior to a treatment (e.g. with an Alzheimer's disease treatment or rapalogue treatment) or prior to an exposure to a genotoxin, DNA-damaging agent, or radiation, for example. The comparisons can also be made to a sample from the same individual from an unaffected area, for example from an unaffected tissue.

One of skill will appreciate that it is often desirable to reduce non-specific binding in immunoassays and during analyte detection. Where the assay involves H1.0K180me2 antibodies or H1.0K180me2 proteins and peptides, immobilized on a solid substrate, it may be desirable to minimize the amount of non-specific binding to the substrate. Methods of reducing such non-specific binding are known to those of skill in the art. Typically, this involves coating the substrate with a proteinaceous composition. In some embodiments, protein compositions such as bovine serum albumin (BSA), nonfat powdered milk, and gelatin may be utilized.

The sensitivity, specificity, positive and negative predictive values (PPV and NPV) as well as positive and negative likelihood ratios (PLR and NLR)) can be calculated for each diagnostic test design. The statistical methods help to predict the presence or absence of disease in the patients.

Sensitivity is generally the probability that the test result will be positive when disease is present (true positive rate).

Specificity is generally the probability that a test result will be negative when the disease is not present (true negative rate).

The positive predictive value (PPV) generally is the probability that the disease is present when the test is positive, accounting for the pre-test prevalence of the disease (e.g. pre-test prevalence for Alzheimer's disease is 10%).

The negative predictive value (NPV) is generally the probability that the disease is not present when the test is negative, accounting for the pre-test prevalence of AD as 10% (Prince, M. J., Am J Epidemiol, 1996).

The positive likelihood ratio (LR+ or PLR) is generally the probability of a person who has the disease testing positive divided by the probability of a person who does not have the disease testing positive. Positive likelihood ratios (PLR) generally indicate how much to increase the probability of the disease, if the test is positive. A PLR>1 indicates an increase probability that the target disorder is present, a PLR<1 indicates a decreased probability that the target disorder is present, and a PLR=1 means that test does not change the probability of the disease.

The negative likelihood ratio (LR- or NLR) is generally the probability of a person who has the disease testing negative divided by the probability of a person who does not have the disease testing negative. Negative likelihood ratios (NLR) generally indicate how much to decrease the probability of the disease, if the test is negative.

In some embodiments, the comparison will be made to a threshold level established by a Receiver Operating Characteristic curve analysis for optimal specificity and sensitivity. The ROC curve, thresholds and areas under the curve (AUC) are shown for each of the test's designs provided herein.

In some embodiments, the diagnostic methods provided herein may be used for confirmatory tests, e.g. to definitively confirm that an individual has a disease, e.g. Alzheimer's disease.

In other embodiments, the diagnostic methods provided herein may be used for their predictive value (for testing, screening), e.g. to determine the likelihood that an individual will develop a disease, e.g. Alzheimer's disease. In such embodiments, the diagnostics may involve computation of likelihood ratios.

In other embodiments, the diagnostic methods provided herein may be used as a companion diagnostic. In such embodiments, the diagnostics may involve computation of positive predictive values (PPV) and negative predictive values (NPV).

In some embodiments, the diagnostic methods provided herein may be used to establish a diagnostic odds ratio (OR). In such embodiments, the diagnostic may involved computation of sensitivity and specificity and is a measure of the effectiveness of a diagnostic test.

### B. Diagnostic H1.0K180 Me2 Antibodies—Direct Detection and Quantification of H1.0K180 Me2

Provided herein are antibodies that specifically bind the H1.0K180me2 antigen, useful for diagnostics. The H1.0K180me2 antibodies provided herein require dimethylation of the K180 residue for binding.

In some embodiments, the H1.0K180me2 antibodies are comprise a label (e.g. conjugated to a label), for example a detectable label, a spin label, a colorimetric label, a radioactive label, an enzymatic label, a fluorescent label, or a magnetic label.

In some embodiments, an antibody is conjugated or attached to a solid surface, for example a bead (e.g. a magnetic, glass or plastic bead), column, resin or a microplate. In some embodiments, an antibody is coated onto the microplate.

H1.0K180me2 antibodies are discussed in more detail in the preceding Section II.

Direct detection is schematically illustrated in FIG. 5A.

### C. Diagnostic H1.0K180 Me2 Proteins and Peptides—Indirect Detection and Quantification of H1.0K180 Me2

Provided herein are H1.0k180me2 proteins and H1.0k180me2 peptides for the detection of naturally occurring H1.0k180me2-autoantibodies in samples.

It is appreciated that production of the H1.0K180me2 antigen in an organism in response to certain stimuli may give rise to the generation of naturally occurring autoantibodies, specific to the antigen. Thus, in some embodiments of the invention, it may be desirable to assay for these naturally occurring autoantibodies against H1.0K180me2. Detection and measurement of the autoantibodies offers a surrogate measurement for the production of H1.0K180me2.

Provided herein are methods and compositions for the detection and measurement of naturally occurring autoantibodies specific to H1.0K180me2 antigen. As used herein, autoantibodies specific to an H1.0K180me2 protein or fragment thereof may be interchangeably referred to as H1.0K180me2 autoantibodies.

The autoantibodies measured may be of any immunoglobulin type such as IgG, IgM, IgE, IgD, or IgA. In some embodiments, IgG autoantibodies against H1.0K180me2 are measured. In some embodiments, IgM autoantibodies against H1.0K180me2 are measured. In some embodiments, more than one type of autoantibody against H1.0K180me2 is measured; for example, in some embodiments, IgG and IgM autoantibodies against H1.0K180me2 are measured. In some embodiments, more than one type of autoantibody against H1.0K180me2 is measured and a ratio is calculated; for example, in some embodiments, IgG and IgM autoantibodies against H1.0K180me2 are measured, and the ratio of IgG autoantibodies against H1.0K180me2: IgM autoantibodies against H1.0K180me2 is calculated. In other embodiments, the ratio of H1.0K180me2 IgM autoantibodies: total IgM (e.g. serum IgM) is measured. In other embodiments, the ratio of H1.0K180me2 IgM autoantibodies: total IgM (e.g. serum IgM) is measured and related to the ratio of H1.0K180me2 IgG autoantibodies: total IgG (e.g. serum IgG) as a function. In some embodiments, more than one type of autoantibody against H1.0K180me2 is measured and compared to transferrin, ferritin or serum albumin content. In other embodiments, more than one type of autoantibody against H1.0K180me2 is measured and normalized to the total amount of protein in the sample or normalized to the volume of the sample.

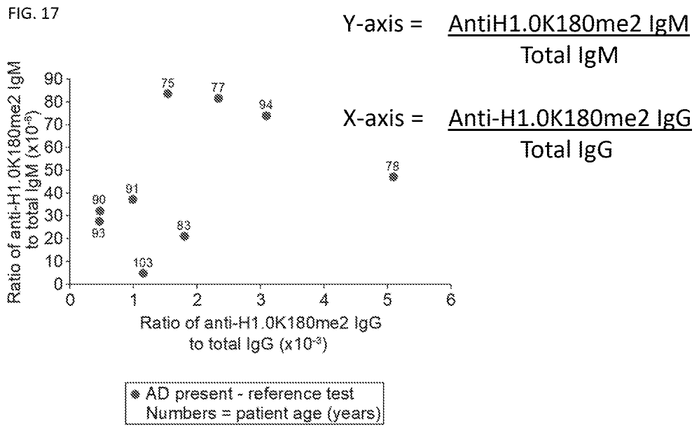

In an exemplary embodiment, the screening test for Alzheimer's disease comprises measuring the ratio of H1.0K180me2 IgM autoantibodies: total IgM (FIG. 17). In another exemplary embodiment, the screening test for Alzheimer's disease comprises measuring the ratio of H1.0K180me2 IgG autoantibodies: total IgG. (FIG. 17). In some embodiments, these are compared to each other to stratify patient populations. (FIG. 17).

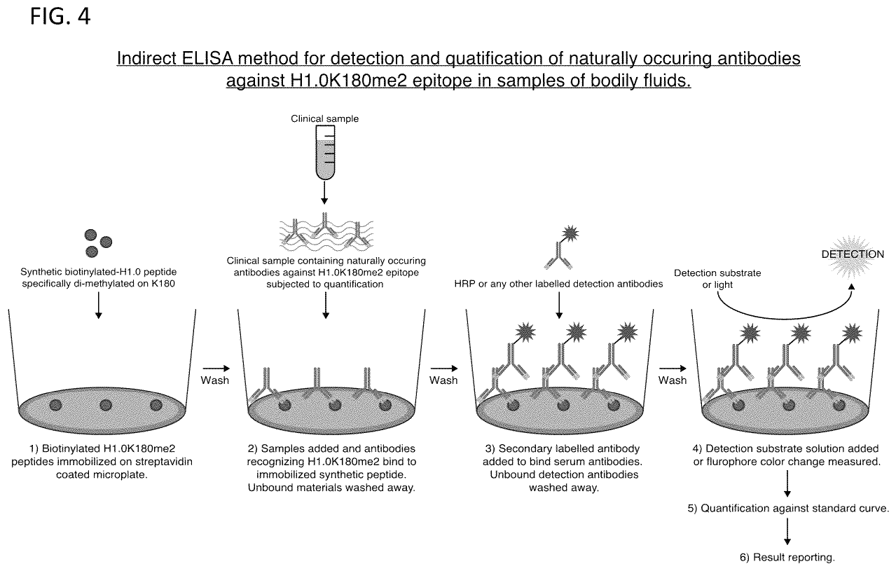

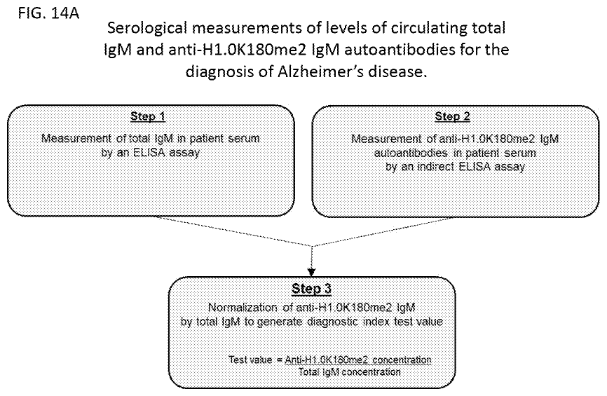

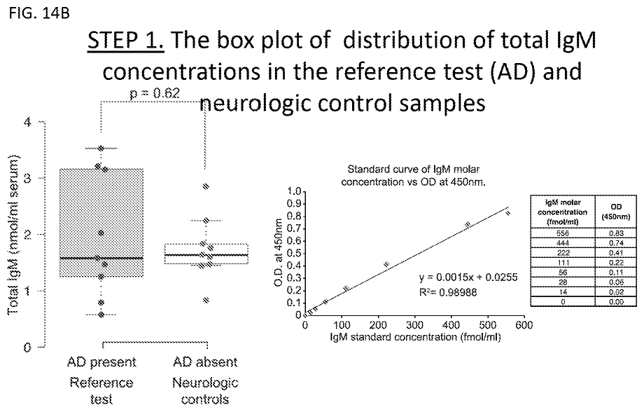

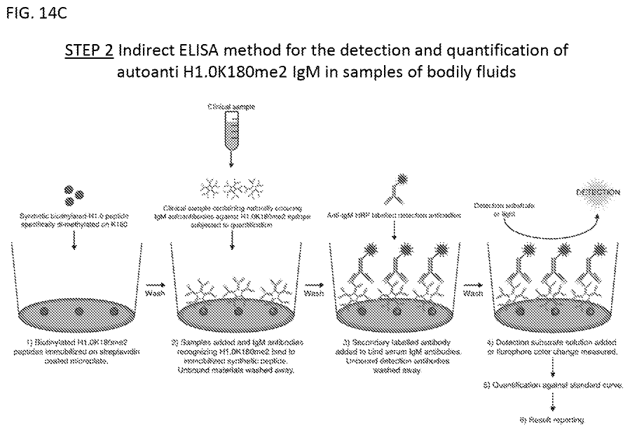

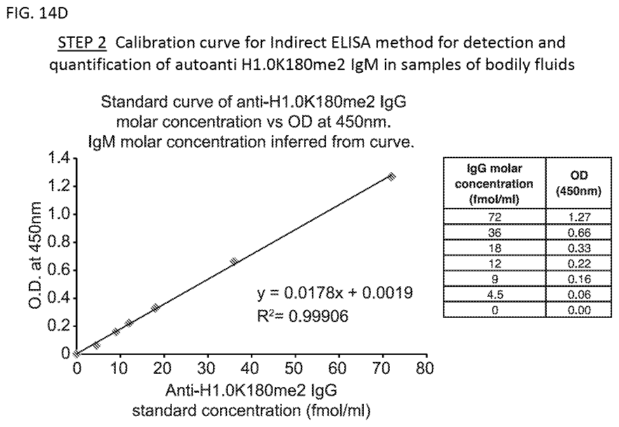

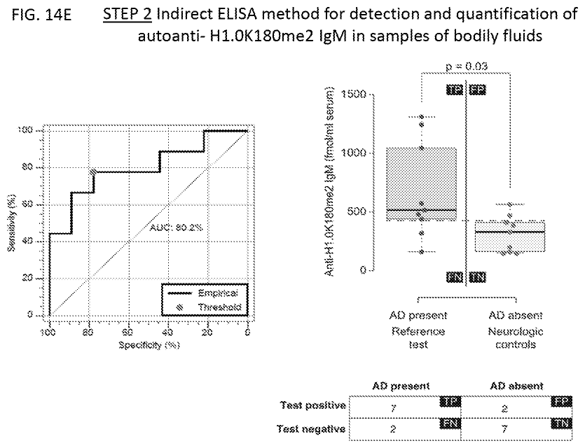

Turning to a schematic presentation, FIGS. 4 and 14C show a method of indirectly measuring H1.0K180me2 levels. As FIG. 4 and FIG. 14C exemplify, labeled H1.0K180me2 peptides are contacted with samples; autoantibodies in the sample generated in response to H1.0K180me2 bind to the labeled peptides; secondary labeled antibody is added to bind the autoantibodies in the sample, followed by detection, and quantification. In FIG. 4, the autoantibodies in the sample can be of any type (IgG, IgM, IgE, IgD, or IgA). In this assay, the secondary antibody used to bind the autoantibodies can distinguish between IgG, IgM, IgE, IgD, and IgA antibodies, such that each type can be independently quantified if so desired. In some embodiments, the secondary antibody is an anti-IgG antibody, and the assay is used to quantify IgG autoantibodies against H1.0K180me2. In some embodiments, the secondary antibody is an anti-IgM antibody, and the assay is used to quantify IgM autoantibodies against H1.0K180me2 (FIG. 14C). In some embodiments, two types of secondary antibodies are utilized to quantify more than one type of autoantibody; for example in some embodiments, both anti-IgG and anti-IgM secondary antibodies are utilized, and the assay is used to quantify IgG and IgM autoantibodies against H1.0K180me2.

In some embodiments, the H1.0K180me2 protein comprises an amino acid sequence of SEQ ID NO:1 or SEQ ID NO:2. In some embodiments, the H1.0K180me2 peptide comprises an amino acid sequence selected from those provided in Table 3A (SEQ ID NOs:3-35). In some embodiments, the H1.0K180me2 peptide comprises the amino acid sequence AKPVKASKPKKAKPVK^{(me2)}PK (SEQ ID NO:3). In some embodiments, the amino acid sequence further comprises an additional terminal residue, for example, for the purpose of conjugation. In some embodiments, the amino acid sequence further comprises a terminal C residue. In such embodiments, the H1.0K180me2 peptide may comprise the amino acid sequence CAKPVKASKPKKAKPVKPK (SEQ ID NO:36), CAKPVKASKPKKAKPVKPKC(SEQ ID NO:37), AKPVKASKPKKAKPVKPKC (SEQ ID NO:38), CAKPVKASKPKKAKPVK^{(me2)}PK (SEQ ID NO:39), CAKPVKASKPKKAKPVK^{(me2)}PKC(SEQ ID NO:40), or AKPVKASKPKKAKPVK^{(me2)}PKC (SEQ ID NO:41). In some embodiments, the H1.0K180me2 peptide comprises a fragment of any one of the H1.0K180me2 peptides provided herein. In some embodiments, the H1.0K180me2 peptide is conjugated to KLH (keyhole limpet hemocyanin), OVA (ovalbumin), BC (bacterial cellulose) or BSA (bovine serum albumin).

In some embodiments the H1.0K180me2 protein or peptide is synthetic, e.g. the product of in vitro methylation, for example with the use of a G9A methyltransferase enzyme, or a G9A-like protein (GLP) methyltransferase enzyme, under conditions that allow for the specific dimethylation of the K180 residue (described herein).

The H1.0K180me2 proteins and peptides provided herein may be further conjugated for a variety of purposes including, but not limited to, for use in detection, diagnostics, visualization, quantification, sorting, therapeutics, and biological assays. In some embodiments, an H1.0K180me2 protein or peptide comprise a label (e.g. conjugated to a label), for example a detectable label, a spin label, a colorimetric label, a radioactive label, an enzymatic label, a fluorescent label, or a magnetic label. In an exemplary embodiment, an H1.0K180me2 protein or peptide is biotinylated. In some embodiments, an H1.0K180me2 protein or peptide is conjugated or attached to a solid surface, for example a bead (e.g. a magnetic, glass or plastic bead), column, or a microplate. In some embodiments, an H1.0K180me2 protein or peptide is coated onto the microplate. In some embodiments, an H1.0K180me2 protein or peptide is conjugated to or comprises an effector molecule including, but not limited to, a radionuclide, a cytotoxin, a chemotherapeutic agent, a drug, a pro-drug, a toxin, an enzyme, an immunomodulator, a pro-apoptotic agent, a cytokine, a hormone, an oligonucleotide, an antisense molecule, a siRNA, and a second antibody.

In some embodiments an H1.0K180me2 protein or peptide is selective for an autoantibody that is specific for H1.0K180me2. In some embodiments, the protein or peptide binds an autoantibody that is non-specific for H1.0K180me2.

In certain embodiments, an H1.0K180me2 protein or peptide provided herein binds the target autoantibody, and has a dissociation constant (Kd) of range of 0.0001 nM to 1 μM. For example, Kd of an H1.0K180me2 protein or peptide may be about 1 μM, about 100 nM, about 50 nM, about 10 nM, about 5 nM, about 1 nM, about 0.5 nM, about 0.1 nM, about 0.05 nM, about 0.01 nM, about 0.005 nM, about 0.001 nM, about 0.0005 nM, or even about 0.0001 nM.

In some embodiments, an H1.0K180me2 protein or peptide is specific for the human H1.0K180me2 autoantibody. In some embodiments, an H1.0K180me2 protein or peptide is cross reactive with an H1.0K180me2 autoantibody from other species.

In some embodiments, an H1.0K180me2 protein or peptide is selective for the H1.0K180me2 autoantibody and exhibits little or no binding to H1.0K180me1 or H1.0K180me3 autoantibodies. In some embodiments, an H1.0K180me2 protein or peptide binds the H1.0K180me2 autoantibody but also exhibits binding to H1.0K180me1 and/or anti-H1.0K180me3 autoantibodies.

In some embodiments, the binding preference of an H1.0K180me2 protein or peptide (e.g., affinity) for the H1.0K180me2 autoantibody is generally at least about 2-fold, about 5-fold, or at least about 10-, 20-, 50-, 10²-, 10³-, 10⁴, 10⁵, or 10⁶-fold over a non-specific autoantibody (e.g. an autoantibody directed to another target).

It is also possible to evaluate an H1.0K180me2 autoantibody protein or peptide to determine whether it has specificity and/or selectivity for the H1.0K180me2 antibody, using methods familiar to those of skill in the art.

Provided herein are nucleic acids encoding the H1.0K180me2 proteins and peptides described herein. Also provided herein are vectors comprising any of the nucleic acids encoding for the H1.0K180me2 proteins and peptides described herein.

### D. Detection of Alzheimer's Disease

Provided herein are H1.0K180me2 antibodies (to determine H1.0K180me2 levels) and H1.0K180me2 proteins and peptides (to determine H1.0K180me2 autoantibody levels), for use in the screening of individuals for Alzheimer's disease, for use in identifying whether individuals are at risk for developing Alzheimer's disease, for use in estimating the likelihood of whether individuals will develop Alzheimer's disease, for use in the diagnosis of Alzheimer's disease, for use in the early detection of Alzheimer's disease, for use in the prognosis of Alzheimer's disease, for selecting individuals who may respond to an Alzheimer's disease treatment with an Alzheimer's disease drug or regimen for use in treatment selection/determining treatment options for those diagnosed with Alzheimer's disease, for use in monitoring the treatment of those diagnosed with Alzheimer's disease and receiving ongoing treatment with an Alzheimer's disease drug or regimen, or for use in screening for Alzheimer's disease drugs and regimens.

Also provided herein are H1.0K180me2 proteins and peptides (to determine H1.0K180me2 IgG and H1.0K180me2 IgM autoantibody levels, also referred to as anti- or autoanti-H1.0K180me2 IgG and anti-autoanti-H1.0K180me2 IgM), for use in stratifying patients into distinct populations. In one embodiment, those distinct populations may be those who have a higher likelihood of responding to an immunotherapy-based treatment of Alzheimer's disease versus those who have a lower or no likelihood of responding to an immunotherapy-based treatment of Alzheimer's disease.

FIG. 17 demonstrates that the simultaneous measurements of anti-H1.0K180me2 IgG and anti-H1.0K180me2 IgM autoantibody levels allows for the stratification of those with Alzheimer's disease into distinct subpopulations, based on the correlation between anti-H1.0K180me2 IgM normalized by total IgM with anti-H1.0K180me2 IgG normalized by total IgG in patient serum.

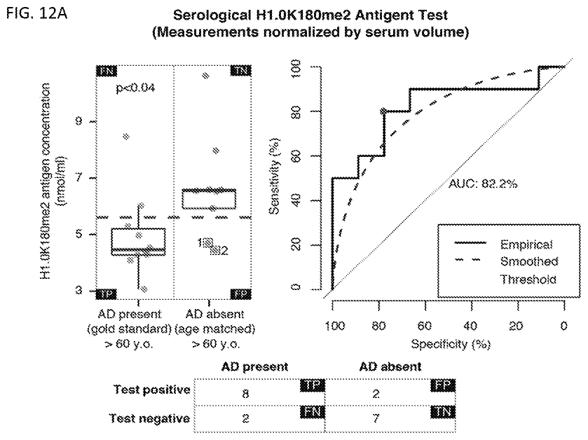

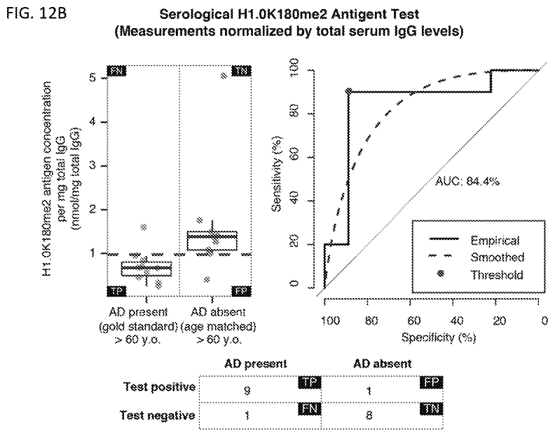

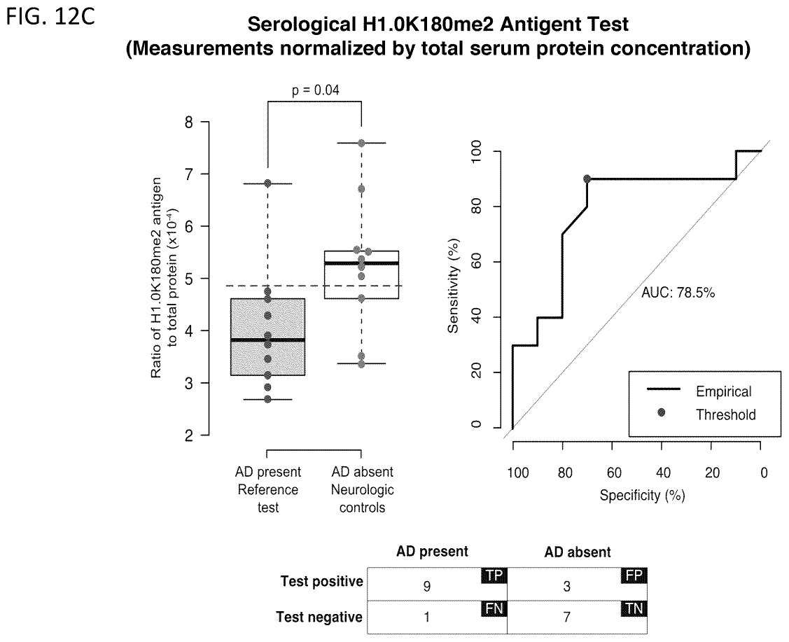

These uses are provided based on the observation that the levels of H1.0K180me2, IgG autoantibodies to H1.0K180me2, and IgM autoantibodies to H1.0K180me2 are altered in patients with Alzheimer's disease. (FIGS. 12A-12C, FIG. 13B, FIGS. 14A-14E, and FIG. 15A-15B).

Alzheimer's disease patients displayed lower concentrations of H1.0K180me2 than healthy age-matched healthy controls, indicating that H1.0K180me2 concentrations may effectively segregate patients with Alzheimer's disease from healthy individuals (FIG. 12A). Although serum concentrations of H1.0K180me2 were sufficient for identification of Alzheimer's disease patients, the use of a serum sample normalization by total IgG (FIG. 12B) or total protein (FIG. 12C) allowed for direct comparisons between individuals regardless of variables which may alter overall serum concentration, such as protocol used to obtain serum, operator variability, hydration state of patient and activity state of patient. For example, it was observed that H1.0K180me2 serum levels were elevated in healthy aged individuals relative to healthy younger individuals, while patients with Alzheimer's disease exhibited significantly lower normalized serum levels of H1.0K180me2 relative to healthy aged, individuals (>60 years) (FIG. 12B, 12C).

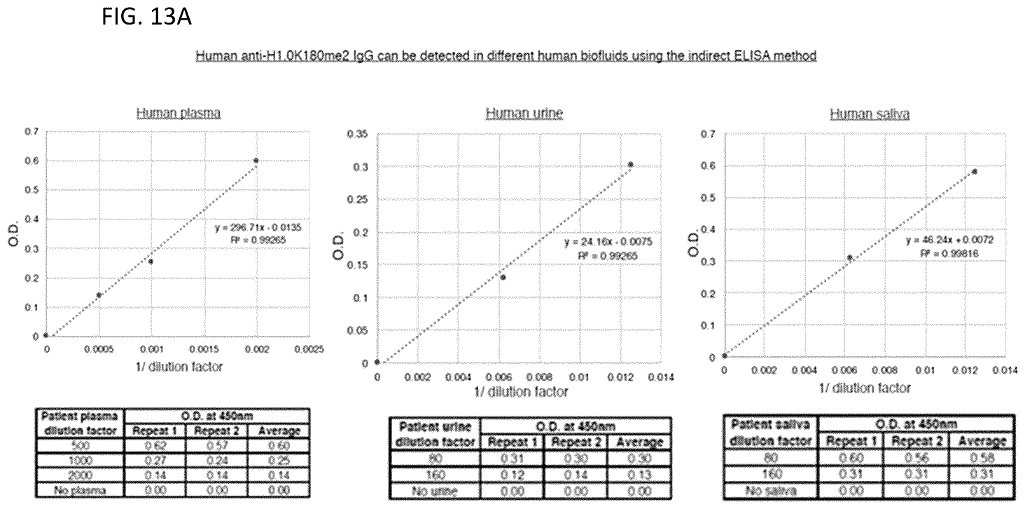

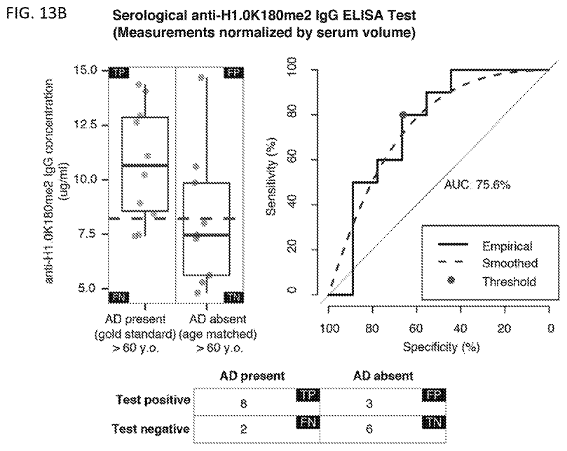

Alzheimer's disease patients also displayed altered levels of H1.0K180me2 autoantibodies, compared to age-matched healthy controls, indicating that anti-H1.0K180me2 IgG and/or IgM serum concentrations (H1.0K180me2 IgG and/or IgM autoantibody concentrations) may effectively segregate patients with Alzheimer's disease from healthy individuals. Alzheimer's disease patients displayed higher concentrations of serum anti-H1.0K180me2 IgG than healthy age-matched controls (FIGS. 13, 14A-E, 15A, 15B). Anti-H1.0K180me2 IgG and IgM levels in human serum were quantified by indirect ELISA analysis using biotinylated-H1.0K180me2 capture peptide (an H1.0K180me2 peptide), followed by the appropriate secondary antibody (either directed to IgG or IgM antibodies). Equal volumes of serum from healthy individuals of 30-40 years (n=7) or >60 years (n=9), and individuals with clinically diagnosed Alzheimer's disease of >60 years (n=10) were analyzed.

FIG. 13B shows the quantification of anti-H1.0K180me2 IgG levels determined by indirect ELISA in Alzheimer's disease patients and age-matched controls. The concentration of anti-H1.0K180me2 IgG in each serum sample was calculated using a standard curve created with serial dilutions of an H1.0K180me2 specific antibody included in the ELISA experiment.

FIG. 14E shows the quantification of raw non-normalized anti-H1.0K180me2 IgM levels determined by indirect ELISA in Alzheimer's disease (AD present) patients and age-matched controls (AD absent; neurologic controls).

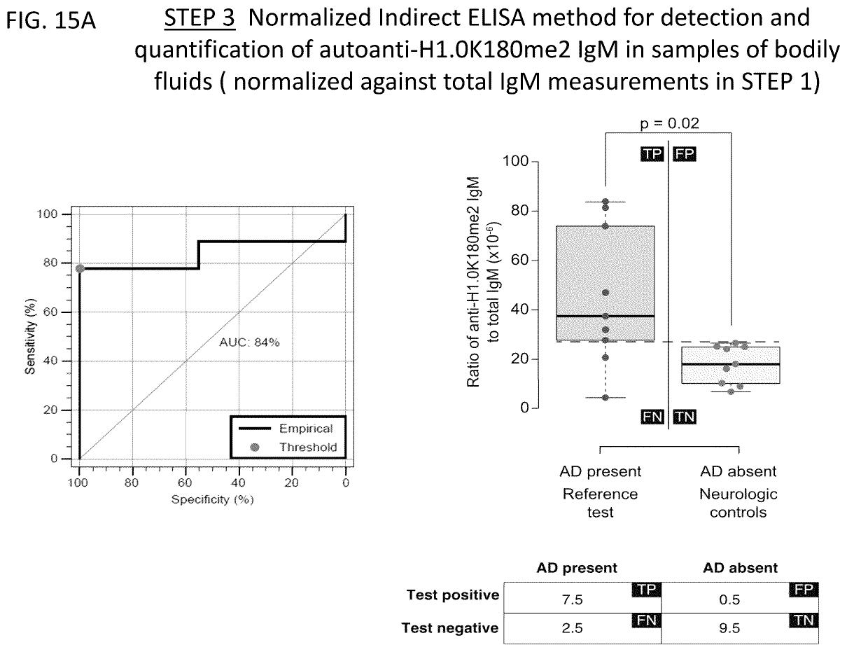

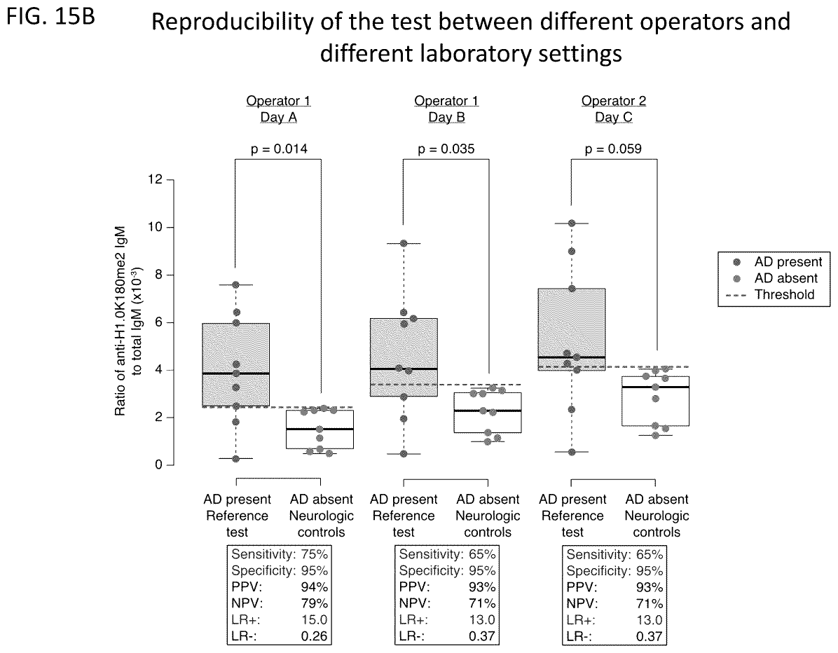

FIGS. 15A and B show the quantification of normalized anti-H1.0K180me2 IgM levels determined by indirect serological ELISA in Alzheimer's disease patients (AD present) and age-matched controls (AD absent; neurologic controls). FIG. 15B demonstrated that diagnostic characteristics do not change regardless of the different test operators and laboratory settings.

Exemplary Methods, H1.0K180me2 Levels: More specifically, in one embodiment, provided herein are methods using an H1.0K180me2 antibody, to determine H1.0K180me2 levels, for screening an individual for Alzheimer's disease, for identifying an individual is at risk for developing Alzheimer's disease, estimating the likelihood of whether an individual will develop Alzheimer's disease, for determining whether an individual has Alzheimer's disease, for detecting the early signs of Alzheimer's disease in an individual, and for use in the prognosis of Alzheimer's disease in an individual, the method comprising: (a) contacting a biological sample from the individual with an H1.0K180me2 antibody; and (b) determining the concentration of H1.0K180me2 in the sample that binds the antibody, wherein a decrease in the concentration relative to a control indicates that the individual has Alzheimer's disease, is at risk of developing Alzheimer's disease, or has a greater likelihood of developing Alzheimer's disease, and wherein an increase or no change in the concentration relative to a control may indicate that the individual does not have Alzheimer's disease, is not at risk of developing Alzheimer's disease, or does not have a greater likelihood of developing Alzheimer's disease. A control includes, but is not limited to, a healthy control (e.g. age-matched, sex-matched), a reference standard representing a compilation of healthy controls, or a control sample from the same individual isolated earlier in time. In some embodiments, the concentration of circulating H1.0K180me2 is determined. In some embodiments, the change in the concentration is relative to a threshold established by a Receiver Operating Characteristic curve analysis for optimal specificity and sensitivity. In some embodiments, the method further comprises treating the individual with an Alzheimer's disease drug or regimen if it is determined that the individual has, or is at risk of developing, Alzheimer's disease.

In one embodiment of practicing the method, a concentration of less than or equal to 5.61 nmol/ml of H1.0K180me2 in the serum indicates the individual has, or may develop Alzheimer's disease. In one embodiment, a concentration of less than or equal to 5.61 nmol/ml of H1.0K180me2 in the serum indicates the individual has, or may develop Alzheimer's disease with a specificity of 78%, a sensitivity 80% and a positive likelihood ratio of 3.6. In one embodiment, this represents a 24% increase in probability (post-test probability) of the Alzheimer's disease compared to pre-test probability. Post-test probability is calculated based on following formula: Pre-test Odds×LR/(1+Pre-test Odds×LR), where pre-test odds are the clinical suspicion of disease presence before testing. The post-test probability is usually calculated from Likelihood Ratio Nomogram or Fagan Nomogram (NEJM 1975; 293:257)

In another embodiment of practicing the method, a ratio of less than or equal to 4.76×10⁻⁴ of H1.0K180me2 to total protein in the serum indicates the individual has, or may develop Alzheimer's disease. In one embodiment, a concentration of less than or equal to 4.76×10⁻⁴ of H1.0K180me2 to total protein in the serum indicates the individual has, or may develop Alzheimer's disease with a specificity of 70%, a sensitivity of 90% and a positive likelihood ratio of 3.00. In one embodiment, this represents a 14.8% increase in probability of the Alzheimer's disease compare to pre-test probability.

Exemplary Methods, H1.0K180me2 Autoantibody Levels: In another embodiment, provided herein are methods using an H1.0K180me2 protein or peptide (to determine H1.0K180me2 autoantibody levels, for example IgG autoantibody levels, or IgM autoantibody levels) for screening an individual for Alzheimer's disease, for identifying an individual is at risk for developing Alzheimer's disease, for estimating the likelihood of whether an individual will develop Alzheimer's disease, for determining whether an individual has Alzheimer's disease, for detecting the early signs of Alzheimer's disease in an individual, and for use in the prognosis of Alzheimer's disease in an individual, the method comprising: (a) contacting a biological sample from the individual with an H1.0K180me2 protein or peptide; and (b) determining the concentration of autoantibodies in the sample that binds the peptide, wherein an increase in the concentration relative to a control indicates that the individual has, or is at risk of developing, Alzheimer's disease, and wherein a decrease, or no change in the concentration relative to a control may indicate that the individual does not have, or is not at risk of developing, Alzheimer's disease. A control can include, but is not limited to, a healthy control (e.g. age-matched, sex-matched), a reference standard representing a compilation of healthy controls, or a control sample from the same individual isolated earlier in time. In some embodiments, the change in the concentration is relative to a threshold established by a Receiver Operating Characteristic curve analysis for optimal specificity and sensitivity. In some embodiments, the method further comprises treating the individual with an Alzheimer's disease drug or regimen if it is determined that the individual has, or is at risk of developing, Alzheimer's disease.

In one embodiment, a concentration of greater than or equal to 9.69 ug/ml (normalized to total IgG levels) of IgG autoantibodies to H1.0K180me2 in the serum indicates the individual has, or may develop Alzheimer's disease. In one embodiment, a concentration of greater than or equal to 9.69 ug/ml of H1.0K180me2 IgG autoantibodies in the serum indicates the individual has, or may develop Alzheimer's disease with a specificity of 89%, a sensitivity of 60% and a positive likelihood ratio of 5.4. In one embodiment, this represents a 30% increase in probability of the Alzheimer's disease compared to pre-test probability.

In some embodiments, the individual has, or is at risk of developing, Alzheimer's disease when the serum concentration of the IgG autoantibodies is greater than or equal to 8.23 ug/ml, normalized to serum volume. In some embodiments, an individual has, or is at risk of developing, Alzheimer's disease when the serum concentration of the IgM autoantibodies is greater than or equal to 409 fMol/ml, normalized to serum volume. In some embodiments, the individual has, or is at risk of developing, Alzheimer's disease when the ratio of the concentration of IgM autoantibodies to total IgM concentration is greater than 26.6×10⁻⁶. In one embodiment, the individual has, or is at risk of developing, Alzheimer's disease when the PLR (LR+) is 2.4 or greater for IgG autoantibodies or the PLR (LR+) is 3.5 or greater for IgM autoantibodies.

In related embodiments, the methods provided herein are used in observational studies. Examples of observational studies include, but are not limited to: (a) Cross-sectional design (single time-point design, or delayed cross-sectional)—testing of one or few specimens/samples per patient that are collected at a single time-point; (b) Longitudinal design—testing of multiple specimens/samples per patient that are collected over an extended period of time (e.g. weeks, months, years); (c) Retrospective design—testing of previously collected specimens for which the analyte status and the patient's clinical status is known (characterized specimens) prior to the commencement of the study; (d) Prospective design—testing of specimens collected before or during the study but for which both the analyte status and the patient's clinical status are established during the study; and (e) Prospective-retrospective design—testing of previously collected specimens for which the clinical status is known but the analyte status is unknown and will be established during the study.

In related embodiments, the methods provided herein are intended for the diagnosis of Alzheimer's disease, wherein the methods may be used to determine, verify or confirm a patient's clinical condition as a sole determinant. In these embodiments, this type of testing also includes sole confirmatory assays (to verify results of previous testing) and sole exclusion assays (to rule out a particular condition).

In related embodiments, the methods provided herein are intended to provide an “aid-to-diagnostic” of Alzheimer's disease, wherein the methods may be used to provide additional information to assist in the determination or verification of a patient's clinical status. The test is not necessarily the sole determinant but may be used to evaluate a patient's current state.

In related embodiments, the methods provided herein are intended for the screening of Alzheimer's disease, wherein the methods may be used to determine the status of a disease, disorder or other physiological state in an asymptomatic individual. Depending on the nature of the condition and the targeted patient population, the screening methods may be used routinely or may be restricted to “at risk” patients. In this context the methods described herein used to evaluate a patient's current state.

In related embodiments, the methods provided herein are intended to determine the predisposition to Alzheimer's disease, wherein the methods described herein may be used to determine the likelihood of disease onset (e.g. assessing the risk of developing the disease in future) in pre-symptomatic patients, where for patients at sufficient risk (as determined by test results), preventive interventions may be taken.

In related embodiments, the methods provided herein are intended for the prognosis of Alzheimer's disease, wherein the methods described herein may be used to measure factors linked to a clinical outcome, irrespective of treatment. The methods described herein may be used to estimate the natural progression of a disease (e.g. outcome in the absence of treatment), or to the methods described herein are designed to evaluate a patient's future state.

In related embodiments, the methods provided herein are intended for determination of physiological status of aging population (“aging clock”) wherein the methods described herein may be used to evaluate the physiological state of an individual for the purpose of identifying a human condition or characteristic of aging or risk of Alzheimer's disease with aging.

In related embodiments, the quantification of H1.0K180me2 antigen and/or quantification of H1.0K180me2 autoantibodies may be used to increase confidence in the Alzheimer's disease screening, diagnosis, or detection.

In related embodiments, the quantification of H1.0K180me2 antigen and/or quantification of H1.0K180me2 autoantibodies may be used in conjunction with other Cerebrospinal Fluids (CSF) tests including but not limited to the measurement of Aß₄₂, T-tau, p-tau, Aß₄₂/T-tau ratio, and Aß₄₂/p-tau.

In related embodiments, the quantification of H1.0K180me2 antigen and/or quantification of H1.0K180me2 autoantibodies may be used in conjunction with assessment of cognitive status tests, for example the MMSE (mini-mental state examination), GDS (global deterioration rate), and CDR (clinical, Dementia rating) tests.

In related embodiments, the quantification of H1.0K180me2 antigen and/or quantification of H1.0K180me2 autoantibodies may be used in conjunction with neuroimaging.

In some embodiments of the methods described herein, the levels of H1.0K180me2 may be normalized against total IgG in the biological sample or normalized against total protein in the biological sample. In some embodiments of the methods described herein, the concentration of H1.0K180me2 may be determined as a relative ratio to non-methylated, labeled, synthetic H1.0 peptide.

In embodiments of the methods described herein, the individual is greater than 50 years old. In some embodiments of the method described herein, the individual is less than 50 years old. In some embodiment, of the method described herein, the individual is at least 50 years old, is at least 55 years old, is at least 60 years old, is at least 65 years old, is at least 70 years old, is at least 75 years old, or is at least 80 years old. In an exemplary embodiment, the individual is at least 60 years old.

### E. Companion Diagnostics for Alzheimer's Disease

Also provided herein are H1.0K180me2 antibodies (to determine H1.0K180me2 levels) and H1.0K180me2 proteins and peptides (to determine H1.0K180me2 autoantibody levels, for example IgG autoantibody levels or IgM autoantibody levels), for use in methods for selecting individuals who may respond to an Alzheimer's disease treatment with an Alzheimer's disease drug or regimen, for use in treatment selection/determining treatment options for those diagnosed with Alzheimer's disease, for use in monitoring the treatment of those diagnosed with Alzheimer's disease and receiving ongoing treatment with an Alzheimer's disease drug or regimen, or for use in screening for Alzheimer's disease drugs and regimens.

In these embodiments, these Alzheimer's disease drugs and regimens/treatments include, but are not limited to, treatment with APP synthesis Inhibitors, beta-secretase inhibitors, gamma-secretase inhibitors and modulators, Aß aggregation inhibitors, Aß immunotherapy, Cholesterol-lowering drugs, Anti-tau drugs, cholinesterase inhibitors, N-methyl D-aspartate (NMDA) antagonists, atypical antipsychotics, blockers of protein S-nitrosylation, glucagon-like peptide-1 receptor agonists, rapamycin, rapalogues, endocannabinoids, cannabinoids, neuroprotectors, molecules controlling calcium influx, antioxidants, anti-inflammatory drugs, drugs controlling control of glutamate homeostasis, autophagy inducers, hormones, hormonal regulators, statins, insulin, insulin carriers, multifunctional nanocarriers, vitamins, nutritional supplements, small RNA molecules, peptides, or ultrasound therapy. More specifically, APP synthesis inhibitors (+phenserine), beta-secretase inhibitors (MK-8931, E2609, LY2811376, LY2886721, PF-05297909, gamma-secretase inhibitors and modulators (semagacestat LY450139, avagacestat BMS-708163, PF-3084014, ELND006, tarenflurbil, CHF5074), Aß aggregation inhibitors (tramiprosate (3APS), clioquinol (PBT1), PBT2, ELND005 (scyllo-inositol), PQ912), Aß immunotherapy (GSK933776, AN1802+QS21, ACC-001, Alzheimer's disease-106, Bapineuzumab, Solanezumab, Gantenerumab (R04909832), Ponezumab (PF-04360365), MABT5102A (crenezumab), BAN2401, Intravenous immunoglobulin, gantenerumab (R1450 or R04909832)), anti-tau drugs (lithium, Tideglusib (NP031112), LMTX (methylene blue)), cholinesterase inhibitors (Razadyne® (galantamine), Exelon® (rivastigmine), and Aricept® (donepezil)), an N-methyl D-aspartate (NMDA) antagonists (Aricept® and Namzaric®, a combination of Namenda® and donepezil), atypical antipsychotics (olanzapine, quetiapine, risperidone), blockers of protein S-nitrosylation, agonist at the glucagon-like peptide-1 receptor, rapamycin and rapalogues, endocannabinoid and cannabinoids, neuroprotectors, molecules controlling calcium influx, antioxidants (Vitamin E, vitamin C, α-lipoic acid, coenzyme Q), anti-inflammatory molecules and drugs, drugs controlling control of glutamate homeostasis, autophagic inducers, hormones and hormonal regulators, statins, insulin and insulin carriers including intranasal insulin, long acting insulin and thalidomide), Ramipril, resveratrol, multifunctional nanocarriers, vitamins and nutritional supplements, small RNA molecules, peptides, and ultrasound therapy. In some embodiments, the Alzheimer's disease drugs and regimens are pending FDA-approval, selected from a list of FDA-registered clinical trials for drug approval, which may be obtained on the world wide web address of the U.S. Food and Drug Administration.

Exemplary Methods, H1.0K180me2 Levels: In one embodiment, provided herein is a method using an H1.0K180me2 antibody for determining whether an individual diagnosed with Alzheimer's disease receiving ongoing treatment, will benefit or continue to benefit from the ongoing treatment, comprising: (a) providing a biological sample from the individual who is receiving ongoing treatment; (b) contacting the biological sample with an H1.0K180me2 antibody; (c) determining the concentration of H1.0K180me2 in the sample that binds the antibody; and (d) selecting an individual who will benefit or continue benefit from treatment, wherein an increase in the concentration relative to a control indicates that the individual will benefit or continue to benefit from treatment, and wherein a decrease or no change in the concentration relative to a control may indicate that the individual will not likely benefit or not continue to benefit from or be responsive to the treatment. A control includes, but is not limited to, samples from individuals with Alzheimer's disease not receiving a treatment, or a control sample from the same individual isolated earlier in time prior to the start of treatment. In some embodiments, the concentration of circulating H1.0K180me2 is determined. In some embodiments, the change in the concentration is relative to a threshold established by a Receiver Operating Characteristic curve analysis for optimal specificity and sensitivity.

Also provided herein is a method using an H1.0K180me2 antibody for use in treatment selection for an individual diagnosed with Alzheimer's disease, for determining treatment options for an individual diagnosed with Alzheimer's disease, and for determining whether who may benefit from a particular treatment. In one embodiment, the method comprises: (a) providing a biological sample from the individual, before the individual is treated for Alzheimer's disease; (b) contacting the biological sample with a candidate treatment; (c) contacting the biological sample with an H1.0K180me2 antibody; (d) determining the concentration and/or subcellular localization of H1.0K180me2 in the sample that binds the antibody; and (e) selecting an individual who may benefit from the treatment, wherein an increase in the concentration relative to a control or a decrease in cytoplasmic subcellular localization indicates that the individual may benefit from a treatment and wherein an decrease or no change in the concentration relative to a control or an increase or no change in cytoplasmic subcellular localization may indicate that the individual will not benefit from a treatment. In another embodiment, the method comprises: (a) administering to the individual the candidate treatment; (b) providing a biological sample from the individual, after administration of the treatment; (c) contacting the biological sample with an H1.0K180me2 antibody; (d) determining the concentration and/or subcellular localization of H1.0K180me2 in the sample that binds the antibody; and (e) selecting an individual who may benefit from the treatment, wherein an increase in the concentration relative to a control or a decrease in cytoplasmic subcellular localization indicates that the individual may benefit from a treatment and wherein an decrease or no change in the concentration relative to a control or an increase or no change in cytoplasmic subcellular localization may indicate that the individual will not benefit from a treatment. A control can include, but is not limited to, samples from healthy individuals, or contacting the biological sample with a placebo treatment. In some embodiments, the concentration of circulating H1.0K180me2 is determined. In some embodiments, the change in the concentration is relative to a threshold established by a Receiver Operating Characteristic curve analysis for optimal specificity and sensitivity.

Exemplary Methods, H1.0K180me2 Autoantibody Levels: In another embodiment, provided herein is a method using an H1.0K180me2 protein or peptide for determining whether an individual diagnosed with Alzheimer's disease receiving ongoing treatment, will benefit or continue to benefit from the ongoing treatment, comprising: (a) providing a biological sample from the individual; (b) contacting the biological sample with an H1.0K180me2 protein or peptide; (c) determining the concentration of autoantibodies in the sample that bind the protein or peptide; and (d) selecting an individual who will benefit or continue to benefit from the treatment, wherein a decrease in the concentration of autoantibodies relative to a control indicates that the individual will benefit or continue to benefit from treatment, wherein no change or an increase in the concentration of autoantibodies relative to a control may indicate that the individual will not benefit or no longer continue to benefit from treatment. A control includes, but is not limited to, samples from individuals with Alzheimer's disease not receiving a treatment, or a control sample from the same individual isolated earlier in time prior to the start of treatment. In some embodiments, the change in the concentration is relative to a threshold established by a Receiver Operating Characteristic curve analysis for optimal specificity and sensitivity. In some embodiments, the concentration of anti-H1.0K180me2 IgG autoantibodies is determined. In some embodiments, the concentration of anti-H1.0K180me2 IgM autoantibodies is determined. In some embodiments, the concentration of anti-H1.0K180me2 IgG and IgM autoantibodies is determined.

Also provided herein is a method using an H1.0K180me2 protein or peptide for use in treatment selection for an individual diagnosed with Alzheimer's disease, for determining treatment options for an individual diagnosed with Alzheimer's disease, and for determining whether the individual may benefit from a particular treatment. In one embodiment, the method may comprise: (a) administering to the individual the candidate treatment; (b) providing a biological sample from the individual after the administration; (c) contacting the sample with an H1.0K180me2 protein or peptide; (d) determining the concentration of autoantibodies in the sample that bind the protein or peptide; and (e) selecting an individual who may benefit from the treatment, wherein a decrease in the concentration of autoantibodies relative to a control indicates that the individual will benefit from the particular treatment, and wherein no change or an increase in the concentration of autoantibodies relative to a control may indicate that the individual will not benefit from the particular treatment. A reference control can include, but is not limited to, samples from healthy individuals, or contacting the biological sample with a placebo treatment. In some embodiments, the change in the concentration is relative to a threshold established by a Receiver Operating Characteristic curve analysis for optimal specificity and sensitivity. In some embodiments, the concentration of anti-H1.0K180me2 IgG autoantibodies is determined. In some embodiments, the concentration of anti-H1.0K180me2 IgM autoantibodies is determined. In some embodiments, the concentration of anti-H1.0K180me2 IgG and IgM autoantibodies is determined.

Also provided herein is a method using an H1.0K180me2 protein or peptide for use stratifying an Alzheimer's disease patients into distinct groups: one group that will likely respond to any type of Alzheimer's disease treatment and one group that will likely not respond to any type of Alzheimer's disease treatment (an may be more restrictive). In one embodiment, the group that will likely respond to any type of Alzheimer's disease treatment, may respond to both immunotherapy and non-immunotherapy-based treatments for Alzheimer's disease. In one embodiment, the group that will likely not respond to just any type of Alzheimer's disease treatment, may respond only to non-immunotherapy-based treatments for Alzheimer's disease. In some embodiments, as provided herein, such stratification may be carried out by calculating the ratio and/or correlating the concentration of anti-H1.0K180me2 IgG antibodies with the concentration of anti-H1.0K180me2 IgM antibodies to fit a statistical model. Such methods may provide viable treatment options for an individual diagnosed with Alzheimer's disease, and for determining whether the individual may benefit from a particular treatment. In one embodiment, the method may comprise: (a) providing a biological sample from the individual; (b) contacting the sample with an H1.0K180me2 protein or peptide; (c) determining the concentration of IgG and IgM autoantibodies in the sample that bind the protein or peptide; and (d) selecting an individual who may benefit from an immunotherapy-based treatment for Alzheimer's disease.

In related embodiments, the methods provided herein are useful for ensuring that the H1.0K180me2 or H1.0K180me2 autoantibody levels remain within physiological levels or within an established therapeutic drug range.

In related embodiments, the methods provided herein are useful for the monitoring of Alzheimer's disease, wherein the methods described may be used to measure the H1.0K180me2 or H1.0K180me2 autoantibody levels for the purpose of adjusting treatments/interventions as required. In related embodiments, the methods provided herein are useful for monitoring the effects of an Alzheimer's disease drug or nutritional regiment or life style adjustment in an individual receiving such treatment.

In related embodiments, the methods provided herein are useful for monitoring the clinical performance in any observational or interventional clinical study for Alzheimer's disease, wherein (a) an observational study refers is a study in which test results obtained during the study are not used for patient management and do not impact treatment decisions; and (b) an interventional study is a study in which test results obtained during the study may influence patient management decisions and may be used to guide treatments.

In related embodiments, the methods provided herein are useful for serial measurement, whereby multiple determinations are taken over time. These types of monitoring methods may be used for the detection/assessment of disease progression/regression, disease recurrence, minimum residual disease, response/resistance to treatment, and/or adverse effects due to treatment. These types of monitoring methods may be designed to evaluate changes in an individual's state.

In related embodiments, the methods provided herein are useful for prediction of Alzheimer's disease treatment response or reaction, wherein the methods described herein may be used to measure factors that determine the likelihood of patient responses or adverse reactions to a specific therapy. Described herein are predictive methods designed specifically for use as companion diagnostics.

In some embodiments of the methods described herein, the levels of H1.0K180me2 may be normalized against total IgG in the biological sample or normalized against total protein in the biological sample. In some embodiments of the methods described herein, the concentration of H1.0K180me2 may be determined as a relative ratio to non-methylated, labeled, synthetic H1.0 peptide.

In embodiments of the methods described herein, the individual is greater than 50 years old. In some embodiments of the method described herein, the individual is less than 50 years old. In some embodiment, of the method described herein, the individual is at least 50 years old, is at least 55 years old, is at least 60 years old, is at least 65 years old, is at least 70 years old, is at least 75 years old, or is at least 80 years old. In an exemplary embodiment, the individual is at least 60 years old.

In related embodiments, the methods may be used for screening for new Alzheimer's disease drugs and regimens.

### F. Detection of Senescence

Provided herein are H1.0K180me2 antibodies and H1.0K180me2 proteins and peptides, for use in detecting senescence. As provided herein, senescence is associated with replicative senescence (REP-SEN), genotoxic stress-induced senescence and radiation-induced senescence.

Generally the method for the detection of senescence involves the direct detection of H1.0K180me2 or indirect detection of H1.0K180me2. For direct detection of H1.0K180me2, the method generally comprises (a) contacting a biological sample from the individual with an H1.0K180me2 antibody; and (b) determining the concentration of the H1.0K180me2 antigen in the sample that binds the antibody, wherein an increase in the concentration relative to a control indicates that the biological sample comprises senescent cells. For indirect detection of H1.0K180me2, the method generally comprises (a) contacting a biological sample from the individual with an H1.0K180me2 protein or peptide; and (b) determining the concentration of the H1.0K180me2 antigen in the sample that binds the peptide, wherein an increase in the concentration relative to a control indicates that the biological sample comprises senescent cells. In some embodiments, the change in the concentration is relative to a threshold established by a Receiver Operating Characteristic curve analysis for optimal specificity and sensitivity.

In some embodiments, the methods may be used for identifying individuals that have undergone senescence induced by a genotoxin. In some embodiments the genotoxic stress-induced senescence is a result of exposure of the individual to a DNA damaging agent, drug or toxin, for example radiation, UV-light, bleomycin and any other genotoxic drugs including but not limited to Trazodone, Etotifen, Cephalexin, Nisoldipme, CGS 15943, Clotrimazole, 5-Nonyltryptamine, Doxepin, Pergolide, Paroxetine, Resveratrol, Quercetin, Honokiol, 7-nitroindazole, Megestrol, Fluvoxamine, Etoposide, Veliparib, Rucaparib, Olaparib, Camptothecin, or Terbinafine and chemotherapeutic drugs in general.

In some embodiments, the methods are useful for identifying senescence in individuals undergoing chemotherapy treatments to ensure that the chemotherapy is effective. The chemotherapeutic agent in these embodiments may be selected from the group consisting of Alemtuzumab (Campath), Alitretinoin (Panretin), Allopurinol (Zyloprim), Altretamine, (Hexalen), Amifostine (Ethyol), Anastrozole (Arimidex), Arsenic Trioxide (Trisenox), Asparaginase (Elspar), BCG Live (TICE BCG), Bexarotene (Targretin), Bleomycin (Blenoxane), Busulfan Intravenous (Busulfex), Busulfan Oral (Myleran), Calusterone (Methosarb), Capecitabine (Xeloda), Streptozocin (Zanosar), Tale (Sclerosol), Tamoxifen (Nolvadex), Temozolomide (Temodar), Teniposide, VM-26 (Vumon), Testolactone (Teslac), Thioguanine, 6-TG (Thioguanine), Thiotepa (Thioplex), and Topotecan (Hycamtin).

### G. Detection of DNA Damage

Provided herein are H1.0K180me2 antibodies and H1.0K180me2 proteins and peptides, for use in detecting DNA damage, for example acute DNA damage.

Generally the method for the detection DNA damage involves the direct detection of H1.0K180me2 or indirect detection of H1.0K180me2. For direct detection of H1.0K180me2, the method generally comprises (a) contacting a biological sample from the individual with an H1.0K180me2 antibody; and (b) determining the concentration of the H1.0K180me2 antigen in the sample that binds the antibody, wherein an increase in the concentration relative to a control indicates that the biological sample has undergone DNA damage. For indirect detection of H1.0K180me2, the method generally comprises (a) contacting a biological sample from the individual with an H1.0K180me2 protein or peptide; and (b) determining the concentration of the H1.0K180me2 antigen in the sample that binds the peptide, wherein an increase in the concentration relative to a control indicates that the biological sample has undergone DNA damage. In some embodiments, the change in the concentration is relative to a threshold established by a Receiver Operating Characteristic curve analysis for optimal specificity and sensitivity.

In some embodiments the DNA damage is a result of exposure of the cells or an individual to a DNA damaging agent, drug or toxin, for example radiation, bleomycin or other DNA-damaging agents (e.g. chemotherapeutic drugs discussed above).

In some embodiments, the method is useful for determining DNA damage within 5 minutes, 10 minutes, 15 minutes, 20 minutes, 25 minutes, 30 minutes, 45 minutes, 60 minutes, 75 minutes, 90 minutes, 2 hours, 3 hours, 4 hours, 5 hours, 24 hours, 48 hours, 3 days, 4 days, or up to 5 days of such exposure to a genotoxin or DNA damaging agent.

In some embodiments, provided herein is a portable unit for the detection of DNA damage. The unit can comprise a sample collection unit, a reader, and an assay module comprising an H1.0K180me2 antibody. The unit can also comprise a sample collection unit, a reader, and an assay module comprising an H1.0K180me2 protein or peptide.

### H. Detection of Radiation Exposure

Provided herein are H1.0K180me2 antibodies and H1.0K180me2 proteins and peptides, for use in detecting radiation exposure.

Generally the method for the detection of radiation exposure involves the direct detection of H1.0K180me2 or indirect detection of H1.0K180me2. For direct detection of H1.0K180me2, the method generally comprises (a) contacting a biological sample from the individual with an H1.0K180me2 antibody; and (b) determining the concentration of the H1.0K180me2 antigen in the sample that binds the antibody, wherein an increase in the concentration relative to a control indicates that radiation exposure has occurred. For indirect detection of H1.0K180me2, the method generally comprises (a) contacting a biological sample from the individual with an H1.0K180me2 protein or peptide; and (b) determining the concentration of the H1.0K180me2 antigen in the sample that binds the peptide, wherein an increase in the concentration relative to a control indicates that radiation exposure has occurred. In some embodiments, the change in the concentration is relative to a threshold established by a Receiver Operating Characteristic curve analysis for optimal specificity and sensitivity. In one exemplary embodiment, the change in the concentration of the H1.0K170me2 antigen after 2 hrs after 7 Gy X-ray exposure is from 12 umol/L to 21 umol/L, and from 26 umol/L to 35 umol/L after 48 hrs.

In some embodiments, the method is useful for determining radiation exposure within 5 minutes, 10 minutes, 15 minutes, 20 minutes, 25 minutes, 30 minutes, 45 minutes, 60 minutes, 75 minutes, 90 minutes, 2 hours, 3 hours, 4 hours, 5 hours, 24 hrs, 48 hrs, 3 days, 4 days, or up to 5 days of such exposure.

In some embodiments, the method is useful determining such exposure in a field situation, for example in a combat zone, with military personnel.

In some embodiments, provided herein is a portable unit for the detection of radiation damage. The unit can comprise a sample collection unit, a reader, and an assay module comprising an H1.0K180me2 antibody. The unit can also comprise a sample collection unit, a reader, and an assay module comprising an H1.0K180me2 protein or peptide.

### I. H1.0K180 Me2 and Rapalogues

The mammalian target of rapamycin (mTOR) has emerged as a promising therapeutic target. Rapamycin and some rapamycin derivatives, rapamycin analogs, and other mTOR inhibitors are FDA-approved drugs for treatment of certain disease states.

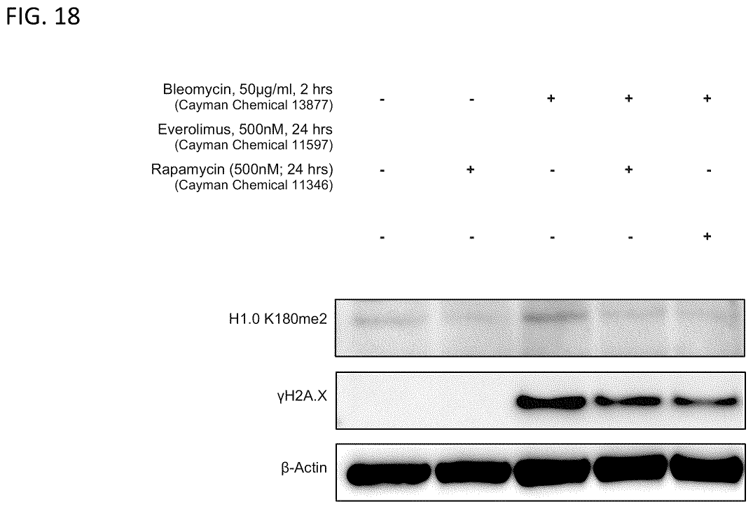

The inventors have found that the detection of H1.0K180me2 may be useful for the screening of an individual's responsiveness to rapamycin, rapamycin derivatives, rapamycin analogs, and other mTOR inhibitors, referred to collectively as “rapalogues,” and may be useful for monitoring rapalogue-based therapeutic regimens. The inventors have also found that the detection of H1.0K180me2 may be useful for drug screening purposes, for the screening of additional rapalogues. Specifically, it is shown here that a rapamycin derivative and immunosuppressant, everolimus, blocks the appearance of H1.0K180me2 upon DNA damage (FIG. 18). It has also been observed that treatment with a rapalogue, prior to, or in parallel with a genotoxic stress exposure, may reduce the effect of the genotoxic stressor, as evidenced by a change in the concentration and/or subcellular localization of H1.0K180me2.

As used herein, rapalogues include FDA-approved rapalogues, and those rapalogues presently undergoing clinical trials. FDA-approved rapalogues include Rapamycin, Sirolimus, Rapamune, Everolimus, RAD001, Afinitor, Zortress, Temsirolimus, CCI-779, Torisel, Ridaforolimus, AP23573, MK-8669, Deforolimus, Zotarolimus, and ABT-578. Other Rapalogues AZD8055, AZD2014, OSI-027, MLN0128, WYE-132, Torin1, PI-103, P7170, PF-04691502, PF-05212384, PKI-587, GNE477, PKI-180, WJD008, XL765, SAR245409, NVP-BEZ235, BGT226, SF1126, GSK2126458, Ku-0063794, WYE-354, NVP-BEZ235, PF-05212384, XL765, Torin 2, WYE-125132, and OSI-027.

Generally provided herein is a method for monitoring the effects of a rapalogue-based treatment ongoing in an individual diagnosed with cancer, an immunodeficiency, diabetes, arthritis, Alzheimer's disease and other neurodegenerative diseases, cardiovascular disease, an autoimmune disease, and other age related pathologies.

Thus, in one embodiment, provided herein is a method of determining whether an individual receiving treatment with a rapalogue is responsive to such treatment, comprising: (a) contacting a biological sample from the individual with an H1.0K180me2 antibody; (b) determining the concentration and/or localization of H1.0K180me2 in the sample that binds the antibody; and (c) determining whether the individual is responsive to treatment, wherein a decrease in the concentration established or a change in the localization relative to a control indicates that the individual is responsive to the rapalogue. In some embodiments, it is determined that the rapalogue treatment is not effective, or needs to be modified if there is an increase in the extracellular concentration of the H1.0K180me2 relative to a control. In some embodiments it is determined that the rapalogue treatment is not effective, or needs to be modified if there is an increase in the cytoplasmic localization of the H1.0K180me2 relative to a control. In some embodiments, the decrease is relative to an age-matched control that is not diagnosed with the relevant disease for which the rapalogue is being administered. In some embodiments it is determined that the treatment is effective, and should be continued if there is a decrease in the extracellular or cytoplasmic concentration of the H1.0K180me2 protein relative to a control. In some embodiments, the method further comprises using the information to modify the treatment type, course, duration, and/or dosage. In some embodiments, the concentration of circulating H1.0K180me2 is determined. In some embodiments, the change in the concentration is relative to a threshold established by a Receiver Operating Characteristic curve analysis for optimal specificity and sensitivity.

Likewise, when the autoantibodies to H1.0K180me2 are used for measurement, the method comprises: (a) contacting a biological sample from the individual with an H1.0K180me2 protein or peptide; (b) determining the concentration of autoantibodies that bind the protein or peptide; and (c) determining whether the individual is responsive to treatment, wherein a change in the concentration of autoantibodies relative to a control indicates that the individual is responsive to the rapalogue. In some embodiments, it is determined that the rapalogue treatment is not effective, or needs to be modified if there is no change in the concentration of the H1.0K180me2 autoantibodies relative to a control. In some embodiments, the decrease is relative to an age-matched control that is not diagnosed with the relevant disease for which the rapalogue is being administered. In some embodiments it is determined that the treatment is effective, and should be continued if there is a change in the concentration of the H1.0K180me2 autoantibodies relative to a control. In some embodiments, the change in the concentration is relative to a threshold established by a Receiver Operating Characteristic curve analysis for optimal specificity and sensitivity.

In some embodiments, provided herein is a method for selecting an individual, diagnosed with or suspected of having cancer, an immunodeficiency, diabetes, Alzheimer's disease or other neurodegenerative diseases, cardiovascular disease, an autoimmune disease, arthritis, and other age related pathologies, who may benefit from rapalogue treatment, or to determine whether an individual may respond to treatment with a rapalogue. In some embodiments, this method comprises providing a biological sample from the individual; treating the sample with a rapalogue in vitro, ex vivo, in slice culture, or in tissue culture; and determining the concentration and/or subcellular localization of H1.0K180me2 in the sample. In some embodiments, the concentration of the H1.0K180me2 is determined, by measuring the concentration of H1.0K180me2 in the sample using an H1.0K180me2 antibody. In some embodiments, the concentration of the H1.0K180me2 is determined, by measuring the concentration of anti-H1.0K180me2 autoantibodies in the sample. In some embodiments it is determined that the rapalogue treatment may not be effective, if there is an increase in the cytoplasmic or extracellular concentration of the H1.0K180me2 relative to a control. In some embodiments it is determined that the rapalogue treatment may not be effective, if there is an increase in the cytoplasmic localization of the H1.0K180me2 relative to a control. In some embodiments, the decrease is relative to an age-matched control that is not diagnosed disease for which the individual may receive a rapalogue treatment. In some embodiments it is determined that the treatment may be effective, if there is a decrease in the cytoplasmic or extracellular concentration of the H1.0K180me2 protein relative to a control. In some embodiments, the change in the concentration is relative to a threshold established by a Receiver Operating Characteristic curve analysis for optimal specificity and sensitivity.

In some embodiments, the methods provided herein further comprises using the information to modify the treatment type, course, duration, and/or dosage.

In other embodiments, provided herein is the use of the detection of H1.0K180me2 to monitor for the ability for new rapalogues to be identified (drug screening applications).

### J. Detection of Biological Aging

Provided herein are H1.0K180me2 antibodies and H1.0K180me2 proteins and peptides, for use in detecting biological aging. As provided herein, biological aging markers or biomarkers of aging are expected to find many uses in biological research since age is a fundamental characteristic of most organisms.

There can be differences between chronological and biological age. Some individuals age more rapidly, while others, due to good habits, genes and/or lack of environmental stressors, age more slowly making them healthier and “younger looking” longer. Being able to track one's biological age may help to modify life style (similar to tracking body mass index) or help to deploy antiaging procedures.

Accurate measures of biological age by aging markers are useful for testing the age-related diseases theories of biological aging, such as (i) diagnosing various age related diseases and for defining cancer subtypes, (ii) predicting/prognosticating the onset of various diseases, and serving as (iii) surrogate markers for evaluating therapeutic interventions including rejuvenation approaches.

Generally the method for the detection of biological aging involves the direct detection of H1.0K180me2 or indirect detection of H1.0K180me2. For direct detection of H1.0K180me2, the method generally comprises (a) contacting a biological sample from the individual with an H1.0K180me2 antibody; and (b) determining the concentration of the H1.0K180me2 antigen in the sample that binds the antibody, wherein an increase in the concentration relative to a control indicates that the biological sample is from an individual that has experienced biological aging. For indirect detection of H1.0K180me2, the method generally comprises (a) contacting a biological sample from the individual with an H1.0K180me2 protein or peptide; and (b) determining the concentration of the H1.0K180me2 antigen in the sample that binds the peptide, wherein an increase in the concentration relative to a control indicates that has experienced biological aging. In some embodiments, the change in the concentration is relative to a threshold established by a Receiver Operating Characteristic curve analysis for optimal specificity and sensitivity.

### K. Diagnostic Kits and Articles of Manufacture

Provided herein are kits useful for the detection of H1.0K180me2. In some embodiments, the kit comprises one or more H1.0K180me2 antibodies or H1.0K180me2 peptides as described herein. In certain embodiments, the antibodies or peptides are labeled. In addition, the kits can optionally include instructional materials for carrying out any of the methods described herein. While the instructional materials typically comprise written or printed materials, they are not limited to such. Any medium capable of storing such instructions and communicating them to an end user is contemplated herein. Such media include, but are not limited to, electronic storage media (e.g., magnetic discs, tapes, cartridges, chips), optical media (e.g., CD ROM), and the like. Such media can include addresses to internet sites that provide such instructional materials.

The kits may also include additional components to facilitate the particular application for which the kit is designed. Thus, for example, where a kit contains an anti-H1.0K180me2 that is labeled, the kit may additionally contain reagents for detecting the label (e.g., enzyme substrates for enzymatic labels, filter sets to detect fluorescent labels, appropriate secondary labels, and the like). The kits may additionally include buffers and other reagents routinely used for the practice of a particular method.

An exemplary kit useful in an immunoassay to detect H1.0K180me2, in addition to an H1.0K180me2 antibody, may include an H1.0K180me2 protein or peptide. This peptide may be employed, for example, as a positive control or as competitor in a competitive immunoassay, and may be labeled or not, depending on the format of the assay to be carried out.

Another exemplary kit useful in an immunoassay to detect H1.0K180me2, in addition to an anti-H1.0K180me2 peptide, will include an H1.0K180me2 antibody. This antibody may be employed, for example, as a positive control or as competitor in a competitive immunoassay, and may be labeled or not, depending on the format of the assay to be carried out.

Also provided herein are transdermal patches for measuring a concentration of a hypodermal target H1.0K180me2 proteins and peptides, comprising: a substrate comprising an H1.0K180me2 antibody; and a plurality of microneedles. In another embodiment, provided herein are transdermal patches for measuring a concentration of H1.0K180me2 autoantibodies, comprising a substrate, and an H1.0K180me2 binding protein or peptide specific for detection of H1.0K180me2 autoantibodies. In some embodiments, the patches are transdermal microneedle array patches. In some embodiments, the substrate of the patches is elastically stretchable. In some embodiments, provided herein are kits comprising patches which comprise the antibodies or peptides provided herein, and optionally, instructions for use. In some embodiments, the patch is useful for detecting and measuring the concentration of H1.0K180me2, or useful for detecting and measuring the concentration of H1.0K180me2 antibodies in a biological sample, for the purpose of detecting replicative senescence, DNA damage, genotoxic stress, radiation exposure, and Alzheimer's disease, useful for monitoring therapeutic regimens, and useful for drug screening.

Also provided herein is a portable unit, for detection, for example for the detection of DNA damage or radiation exposure. The unit may comprise a sample collection unit, a reader, and an assay module comprising an H1.0K180me2 antibody. The unit may also comprise a sample collection unit, a reader, and an assay module comprising an H1.0K180me2 protein or peptide.

Also provided herein are lateral flow strips or test strips suitable for a lateral flow assay of an analyte, comprising a sample receiving zone, wherein the sample receiving zone comprises either an H1.0K180me2 antibody or an anti-H1.0K180me2 binding peptide. In some embodiments, antibody or the peptide comprises a label.

### IV. Therapeutics

### A. Treatment of Methylated H1.0-Related Diseases and Conditions

Provided herein are therapeutic H1.0K180me2 antibodies, therapeutic H1.0K180me2 proteins, and therapeutic H1.0K180me2 peptides for the treatment of a methylated H1.0-related disease or condition.

As used herein, a “methylated H1.0-related disease or condition” is one where there is an increase in the levels of H1.0K180me2, an increase in the endogenous dimethylation of a H1.0 protein/peptide substrate at K180, an increase in the release of H1.0K180me2 from the chromatin, an increase in the release of H1.0K180me2 from the nucleus into the cytoplasm, an increase in the cytoplasmic deposition of H1.0K180me2, an increase in the levels of H1.0K180me2 in the extracellular space, an increase in the circulating levels of H1.0K180me2 in a bodily fluid (e.g. serum, urine, saliva, cerebrospinal fluid, etc.), and/or an increase in the level of autoantibodies specific for H1.0K180me2.

Methylated H1.0-related diseases and conditions include, but are not limited to, age-related pathologies associated with increase of senescent cells, Alzheimer's disease, radiation exposure, exposure to a genotoxic stressor, conditions that comprise the accumulation of senescent cells associated with external and internal stressors, and autoimmune group of diseases and conditions associated with high level of autoantibodies to H1.0K180me2.

Provided herein are methods of treating a methylated H1.0-related disease or condition in an individual by binding and clearing H1.0K180me2 comprising administering to the individual a therapeutically effective amount of a therapeutic H1.0K180me2 antibody.

Also provided herein are methods of treating a methylated H1.0-related disease or condition in an individual to bind and clear H1.0K180me2 autoantibodies comprising administering to the individual a therapeutically effective amount of a therapeutic H1.0K180me2 protein or therapeutic H1.0K180me2 peptide.

As used herein, an individual refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Individuals may be male or female.

In some embodiments of the methods described herein, the individual is greater than 50 years old. In some embodiments of the method described herein, the individual is less than 50 years old. In some embodiment, of the method described herein, the individual is at least 50 years old, is at least 55 years old, is at least 60 years old, is at least 65 years old, is at least 70 years old, is at least 75 years old, or is at least 80 years old. In an exemplary embodiment, the individual is at least 60 years old.

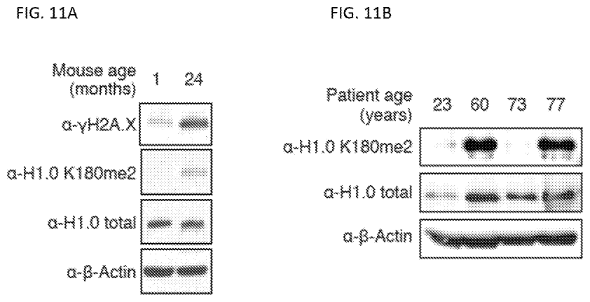

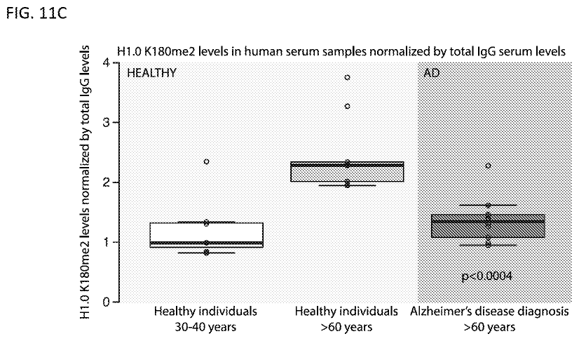

Turning to the methylated H1.0-related diseases and conditions more specifically, during aging, there is increased accumulation of cytoplasmic H1.0K180me2 in the brain tissue of humans FIG. 11B. These observations are correlated with an age-related increase of circulating H1.0K180me2 antigen observed in serological tests (FIG. 11C).

However in individuals diagnosed with Alzheimer's disease, there is a drop in the circulating levels of the H1.0K180me2 antigen (FIG. 11C) observed serological ELISA tests when comparing a control population (no Alzheimer's disease pathology) with a reference control (diagnosed Alzheimer's disease patients) (FIGS. 12A-12C).

This drop in the circulating levels of H1.0K180me2 antigen in the Alzheimer's disease group is associated with an increased level of IgG and IgM autoantibodies to the H1.0K180me2 epitope, when compared to age-matched individuals with no pathology (FIGS. 13B, 14B, 14D, 14E, 15A, 16A, 16B)

Thus in the context of Alzheimer's disease, treatment with a therapeutically effective amount of an H1.0K180me2 antibody (e.g. a cell penetrating antibody or a cell clearing antibody) is provided.

Additionally, naturally occurring serum IgG and IgM autoantibodies generated in response to H1.0K180me2 are elevated in individuals with Alzheimer's disease, when compared to age-matched controls (FIGS. 13B, 14B, 14D, 14E, 15A, 16A, 16B), an may be harmful in attacking and destroying the cells expressing H1.0K180me2. Thus, in this context, treatment with a therapeutically effective amount of an H1.0K180me2 antibody binding (neutralizing) protein or peptide is provided.

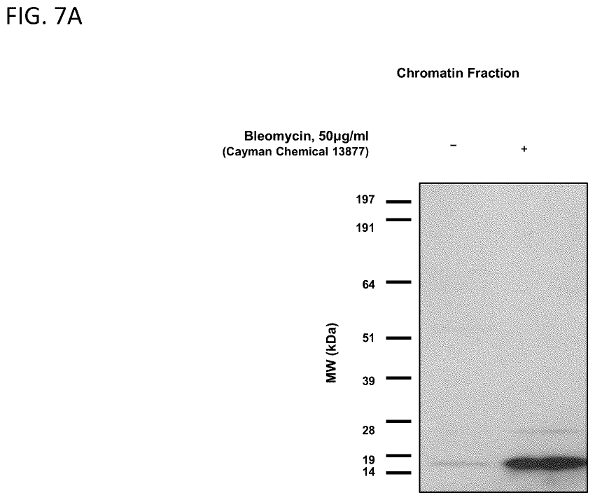

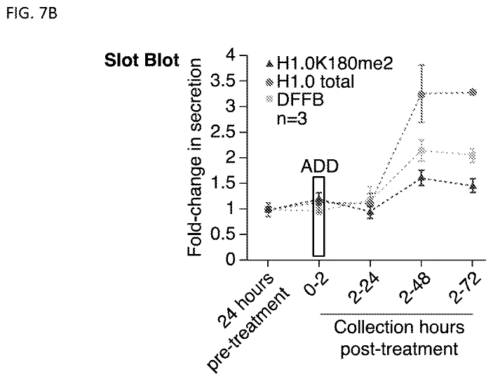

In the context of DNA damaging agents, dimethylation of H1.0K180 (H1.0K180me2) is observed on chromatin following acute DNA damage with bleomycin, a chemotherapeutic agent (FIG. 7A). Thus in this context, treatment with a therapeutically effective amount of an H1.0K180me2 antibody (e.g. a cell penetrating antibody, or a cell-clearing antibody) is provided. Additionally, naturally occurring autoantibodies generated in response to H1.0K180me2 increase in dimethylation of the H1.0 protein provide a target for treatment with a therapeutically effective amount of an H1.0K180me2 antibody binding (neutralizing) protein or peptide.

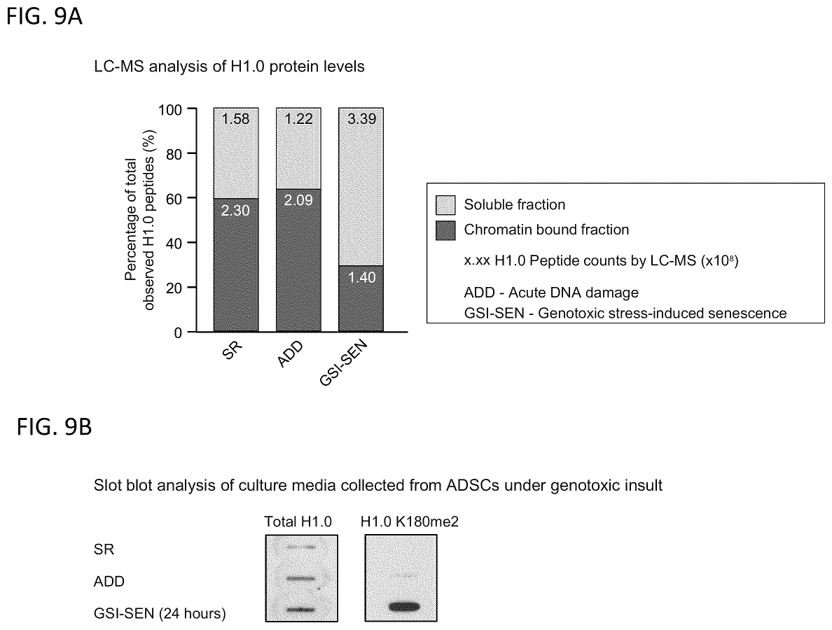

In the context of genotoxic stress, H1.0K180me2 is released from the chromatin upon genotoxic stress induced senescence (days after treatment with a DNA damaging agent) (FIG. 9A) and is secreted out of the cells into the extracellular space (FIG. 9B). Thus in this context, treatment with a therapeutically effective amount of an H1.0K180me2 antibody is provided. Additionally, naturally occurring autoantibodies generated in response to the increase in H1.0K180me2 provide a target for treatment with a therapeutically effective amount of an H1.0K180me2 antibody binding (neutralizing) protein or peptide.

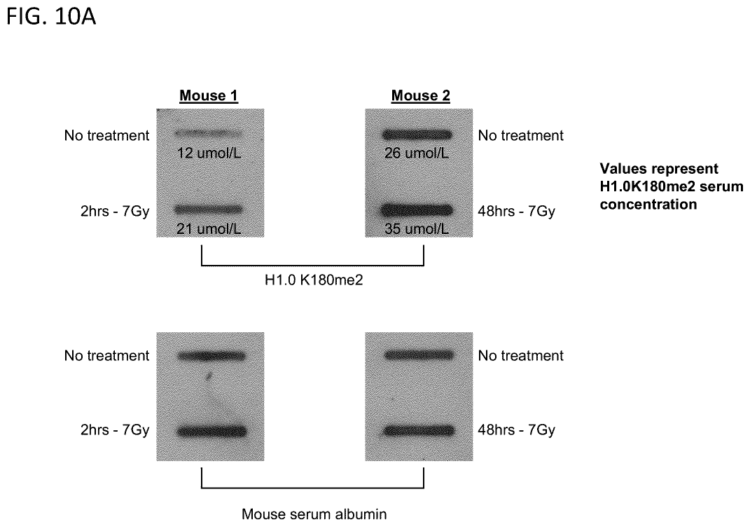

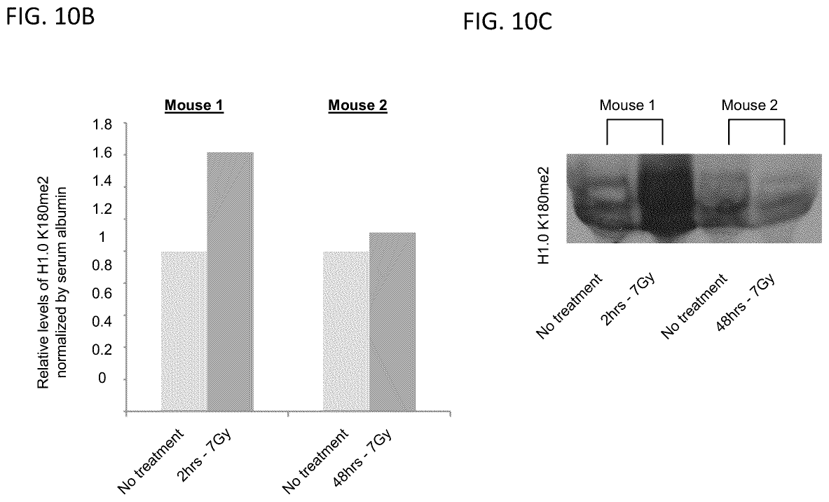

In the context of radiation exposure, exposure to ionizing radiation induces increased levels of circulating H1.0K180me2 in serum (FIG. 10A and FIG. 10B). Thus in this context, treatment with a therapeutically effective amount of an H1.0K180me2 antibody is provided. Additionally, naturally occurring autoantibodies generated in response to the increase in H1.0K180me2 provide a target for treatment with a therapeutically effective amount of an H1.0K180me2 antibody binding (neutralizing) protein or peptide.

In some embodiments, diagnostic methods to determine whether the individual to be treated is indeed afflicted with a methylated H1.0-related disease or condition is carried out. Assays to test for the presence, localization, chromatin fraction, cytoplasmic accumulation, serum levels, autoantibody levels of H1.0K180me2 or H1.0K180me2 antibodies, indicative of a methylated H1.0-related disease or condition, may be carried out in advance of the treatment.

### B. Therapeutic H1.0K180 Me2 Antibodies

As discussed in the above Section (II)(A), provided herein are antibodies that recognize and specifically bind to the H1.0K180me2 epitope, and may be used for therapeutics.

In additional to the H1.0K180me2 antibodies that recognize the dimethylated K180 antigen, in some embodiments, the therapeutic antibody is an anti-idiotypic antibody. Such idiotypic antibodies recognize disease-associated IgG-autoantibodies or disease-associated IgM-autoantibodies in patients with Alzheimer's disease, or other methylated H1.0-related diseases or conditions. In some embodiments, in vitro IgMs (IVIgMs) are generated from the plasma of Alzheimer's disease patient donors and may be used as a preventive therapeutic or vaccination for Alzheimer's disease. Such IVIgMs may contain anti-idiotypic antibodies that recognize disease-associated-autoantibodies in patients. Thus, in some embodiments, the IVIgMs can have idiotypic antibodies where the idiotypic antibody and may inactivate the IgG and/or IgM autoantibodies against H1.0K180me2.

The therapeutic antibodies provided herein may be of any immunoglobulin type such as IgG, IgA, IgE, IgD, or IgM, or anti-idiotypic antibody against IgG, IgA, IgE, IgD, or IgM. In some embodiments, the antibody is of the IgG subtype and may be an IgG1 antibody, an IgG2 antibody, an IgG3 antibody, or a IgG4 antibody. In some embodiments, the antibody is of the IgM subtype.

The antibodies provided herein may be further conjugated for a variety of purposes including, but not limited to, for use in detection, visualization, quantification, sorting, therapeutics, and for use in biological assays, that relate to their therapeutic use.

In some embodiments, the therapeutic antibody is a neutralizing antibody, and the antibody neutralizes one or more biological activities of H1.0K180me2. For example, the antibody may bind extracellular H1.0K180me2 and neutralize any binding or signaling activity it may possess. In some embodiments, the antibody may clear or block H1.0K180me2 in a cell, or a sample. In some embodiments, the antibody may clear the cells comprising H1.0K180me2.

In some embodiments, the therapeutic antibody may clear senescent cells. In some embodiments, the antibody may clear cells/tissue or protein products that give rise to the symptom of Alzheimer's disease. In some embodiments, the antibody may clear cells damaged by radiation damage, DNA damaging agents, and other genotoxins. In some embodiments, the antibody is a cell-penetrating antibody. In other embodiments, the affected cells' membranes are comprised and allow for entry of the therapeutic H1.0K180me2 antibodies provided herein.

In some embodiments, the therapeutic antibody provided herein has antibody-dependent cellular cytotoxicity (ADCC) activity. Effector cells bearing Fc gamma receptors (FcγR or FCGR) on their cell surface, including cytotoxic T-cells, natural killer (NK) cells, macrophages, neutrophils, eosinophils, dendritic cells, or monocytes, recognize and bind the Fc region of antibodies bound to the target-cells. Such binding may trigger the activation of intracellular signaling pathways leading to cell death.

In some embodiments, the therapeutic antibody has complement-dependent cytotoxicity (CDC) activity. Antibody-induced CDC is mediated through the proteins of the classical complement cascade and is triggered by binding of the complement protein Clq to the antibody. Antibody Fc region binding to Clq may induce activation of the complement cascade.

In some embodiments, the therapeutic antibody has antibody-dependent cellular phagocytosis (ADCP) activity. Phagocytic cells bearing Fc receptors on their cell surface, including monocytes and macrophages, recognize and bind the Fc region of antibodies bound to target-cells. Upon binding of the Fc receptor to the antibody-bound target cell, phagocytosis of the target cell may be initiated.

In some embodiments, the therapeutic antibodies may form an immune complex. For example, an immune complex may be a cell expressing or extruding H1.0K180me2 antigen, covered by antibodies.

### C. Therapeutic H1.0K180 Me2 Proteins and H1.0K180 Me2 Peptides

Production of the H1.0K180me2 antigen in an individual afflicted with a methylated H1.0 related-disease or condition gives rise to the generation of naturally occurring autoantibodies specific for the H1.0K180me2 antigen. Thus, the therapeutic approach of binding and clearing these naturally occurring autoantibodies is provided herein. For this purpose, herein are provided therapeutic H1.0K180me2 proteins and therapeutic H1.0K180me2 peptides retaining the dimethylated lysine corresponding to K180, which may bind, and in some embodiments additionally neutralize, the naturally occurring autoantibodies generated in response to the H1.0K180me2 epitope in cells or in circulation.

In some embodiments, the therapeutic H1.0K180me2 proteins/peptides may block the biological actions of H1.0K180me2 autoantibodies, in the serum of an individual. Such blockade in autoantibody levels may lessen the immune response, and may ameliorate the adverse symptoms of the methylated H1.0 disease or condition.

In one embodiment, an therapeutic H1.0K180me2 protein is the H1.0K180me2 full length protein (SEQ ID NO:1). The therapeutic H1.0K180me2 peptides provided herein may be any fragment of a full length H1.0K180me2 protein, retaining the dimethylated K180 reside. In some embodiments the therapeutic H1.0K180me2 peptides range from 5-193 amino acids (aa) long. In some embodiments, the length of an therapeutic H1.0K180me2 peptide is 5aa, 6aa, 7aa, 8aa, 9aa, 10aa, 11aa, 12aa, 13aa, 14aa, 15aa, 16aa, 17aa, 18aa, 19aa, 20aa, 21aa, 22aa, 23aa, 24aa, 25aa, 26aa, 27aa, 28aa, 29aa, or even 30aa. In certain exemplary embodiments, the length of the therapeutic H1.0K180me2 peptide is 15aa, 16aa, 17aa, 18aa, 19aa, or 20aa. Table 3A provides exemplary sequences of the therapeutic H1.0K180me2 peptides provided herein. Thus, in some embodiments, the therapeutic H1.0K180me2 peptide comprises one of the sequences selected from those presented in Table 3A. In related embodiments, the therapeutic H1.0K180me2 peptide consists of one of the sequences selected from those presented in Table 3A. In an exemplary embodiment, the therapeutic H1.0K180me2 peptide comprises the sequence of SEQ ID NO:3, SEQ ID NO:4, or SEQ ID NO:5. In an exemplary embodiment, the H1.0K180me2 antibody-binding peptide consists of the sequence of SEQ ID NO:3, SEQ ID NO:4, or SEQ ID NO:5.

In some embodiments the therapeutic H1.0K180me2 protein or peptide is synthetic. In some embodiments, the therapeutic H1.0K180me2 protein or peptide is the product of an in vitro methylation reaction, for example with the use of a G9A methyltransferase enzyme, or a G9A-like protein (GLP) methyltransferase enzyme, under conditions that allow for the specific dimethylation of the K180 residue, described in greater detail herein.

As provided for in Section I, therapeutic H1.0K180me2 proteins/peptides provided herein may be further conjugated for a variety of purposes including, but not limited to, for use in therapeutics, and for use in biological assays related to their therapeutic use.

In some embodiments, therapeutic H1.0K180me2 proteins/peptides are comprises a label (e.g. are conjugated to a label), for example a detectable label, a spin label, a colorimetric label, a radioactive label, an enzymatic label, a fluorescent label, or a magnetic label. In an exemplary embodiment, therapeutic H1.0K180me2 proteins/peptides are biotinylated. In some embodiments, therapeutic H1.0K180me2 proteins/peptides are conjugated or attached to a solid surface, for example a bead (e.g. a magnetic, glass or plastic bead), column, or a microplate. In some embodiments, therapeutic H1.0K180me2 proteins/peptides are coated onto the microplate. In some embodiments, therapeutic H1.0K180me2 proteins/peptides are conjugated to or comprises an effector molecule including, but not limited to, a radionuclide, a cytotoxin, a chemotherapeutic agent, a drug, a pro-drug, a toxin, an enzyme, an immunomodulator, a pro-apoptotic agent, a cytokine, a hormone, an oligonucleotide, an antisense molecule, a siRNA, and a second antibody.

In some embodiments therapeutic H1.0K180me2 proteins/peptides are selective for an autoantibody that is specific for H1.0K180me2. In some embodiments, the therapeutic H1.0K180me2 proteins/peptides bind an autoantibody that is non-specific for H1.0K180me2.

In certain embodiments, therapeutic H1.0K180me2 proteins/peptides provided herein have a dissociation constant (Kd) of range of 0.0001 nM to 1 μM. For example, the Kd of an anti-H1.0K180me2 binding peptide may be about 1 μM, about 100 nM, about 50 nM, about 10 nM, about 5 nM, about 1 nM, about 0.5 nM, about 0.1 nM, about 0.05 nM, about 0.01 nM, about 0.005 nM, about 0.001 nM, about 0.0005 nM, or even about 0.0001 nM.

In some embodiments, therapeutic H1.0K180me2 proteins/peptides are specific for the human H1.0K180me2 autoantibody. In some embodiments, therapeutic H1.0K180me2 proteins/peptides are cross reactive with an H1.0K180me2 autoantibody from other species.

In some embodiments, therapeutic H1.0K180me2 proteins/peptides are selective for the H1.0K180me2 autoantibody and exhibit little or no binding to autoantibodies that bind H1.0K180me1 or H1.0K180me3. In some embodiments, therapeutic H1.0K180me2 proteins/peptides bind the H1.0K180me2 autoantibody but also exhibits binding to H1.0K180me1 and/or H1.0K180me3 autoantibodies.

In some embodiments, the binding preference of therapeutic H1.0K180me2 proteins/peptides (e.g., affinity) for the H1.0K180me2 antibody is generally at least about 2-fold, about 5-fold, or at least about 10-, 20-, 50-, 10²-, 10³-, 10⁴, 10⁵, or 10⁶-fold over a non-specific target antibody (e.g. a randomly generated antibody).

It is also possible to evaluate therapeutic H1.0K180me2 proteins/peptides to determine whether they have specificity and/or selectivity for the H1.0K180me2 antibody, using methods familiar to those with skill in the art.

Provided herein are nucleic acids encoding the therapeutic H1.0K180me2 peptides described herein. Also provided herein are vectors comprising any of the nucleic acids encoding for the therapeutic H1.0K180me2 peptides described herein.

### D. Combination Therapies

The administration of any of the therapeutic H1.0K180me2 antibodies and the therapeutic H1.0K180me2 proteins/peptides provided herein may be administered in combination with other known drugs/treatments for the diseases which manifest in H1.0 methylation.

In the context of Alzheimer's disease prevention, any of the therapeutic H1.0K180me2 antibodies may be administered in combination with the administration of an Alzheimer's disease preventive drug or regimen which include, but are not limited to, APP synthesis Inhibitors, beta-secretase inhibitors, gamma-secretase inhibitors and modulators, Aß aggregation inhibitors, Aß immunotherapy, Cholesterol-lowering drugs, Anti-tau drugs, cholinesterase inhibitors, N-methyl D-aspartate (NMDA) antagonists, atypical antipsychotics, blockers of protein S-nitrosylation, glucagon-like peptide-1 receptor agonists, rapamycin, rapalogues, endocannabinoids, cannabinoids, neuroprotectors, molecules controlling calcium influx, antioxidants, anti-inflammatory drugs, drugs controlling control of glutamate homeostasis, autophagy inducers, hormones, hormonal regulators, statins, insulin, insulin carriers, multifunctional nanocarriers, vitamins, nutritional supplements, small RNA molecules, peptides, and ultrasound therapy.

In context of Alzheimer's disease treatment any of the therapeutic an therapeutic H1.0K180me2 proteins/peptides may be administered in combination with the administration of an Alzheimer's disease drug or regimen which include, but are not limited to, APP synthesis Inhibitors, beta-secretase inhibitors, gamma-secretase inhibitors and modulators, Aß aggregation inhibitors, Aß immunotherapy, Cholesterol-lowering drugs, Anti-tau drugs, cholinesterase inhibitors, N-methyl D-aspartate (NMDA) antagonists, atypical antipsychotics, blockers of protein S-nitrosylation, glucagon-like peptide-1 receptor agonists, rapamycin, rapalogues, endocannabinoids, cannabinoids, neuroprotectors, molecules controlling calcium influx, antioxidants, anti-inflammatory drugs, drugs controlling control of glutamate homeostasis, autophagy inducers, hormones, hormonal regulators, statins, insulin, insulin carriers, multifunctional nanocarriers, vitamins, nutritional supplements, small RNA molecules, peptides, and ultrasound therapy.

### E. Administration of the Therapeutic H1.0K180 Me2 Antibodies and Therapeutic Anti-H1.0K180 Me2 Binding Proteins/Peptides

In vivo administration of the therapeutic H1.0K180me2 antibodies and therapeutic H1.0K180me2 proteins/peptides described herein may be carried out intravenously, intramuscularly, subcutaneously, topically, orally, transdermally, intraperitoneally, intraorbitally, by implantation, by inhalation, intrathecally, intraventricularly, or intranasally. An effective amount of the therapeutic may be administered for the treatment of the disease or condition manifesting in H1.0 methylation, or a disease or condition manifesting elevated levels of H1.0K180me2 autoantibodies. The appropriate dosage of the therapeutic may be determined based on the type of disease or disorder to be treated, the type of the therapeutic antibody, protein, or peptide, the severity and course of the disease or condition, the clinical condition of the individual, the individual's clinical history and response to the treatment, and the discretion of the attending physician.

For in vivo administration of the therapeutic H1.0K180me2 antibodies and therapeutic H1.0K180me2 proteins/peptides described herein, normal dosage amounts may vary from about 1 ng/kg up to about 1000 mg/kg of an individual's body weight or more per day, depending upon the route of administration. For repeated administrations over several days or longer, depending on the severity of the methylated H1.0-related disease or condition to be treated, the treatment may be sustained until a desired suppression of symptoms is achieved. Dosage regimens may be useful, depending on the pattern of pharmacokinetic decay that the physician wishes to achieve. For example, dosing an individual from one to twenty-one times a week is provided herein. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is may be monitored by conventional techniques and assays. The dosing regimen may vary over time independently of the dose used.

### F. Pharmaceutical Compositions

The present application provides compositions comprising therapeutic H1.0K180me2 antibodies and therapeutic H1.0K180me2 proteins/peptides, including pharmaceutical compositions comprising any one or more of the therapeutic antibodies, proteins or peptides described herein with one or more pharmaceutically acceptable excipients. In some embodiments the composition is sterile. The pharmaceutical compositions generally comprise an effective amount of the therapeutic antibody, protein, or peptide.

### G. Kits and Articles of Manufacture

The present application provides kits comprising a therapeutic H1.0K180me2 antibody, a therapeutic H1.0K180me2 protein, and therapeutic H1.0K180me2 peptide compositions described herein. In some embodiments, the kits further contain a component selected from any of secondary antibodies, reagents for immunohistochemistry analysis, pharmaceutically acceptable excipient and instruction manual and any combination thereof. In one embodiment, the kit comprises any one or more of the therapeutic compositions described herein, with one or more pharmaceutically acceptable excipients.

The present application also provides articles of manufacture comprising any one of the therapeutic compositions or kits described herein. Examples of an article of manufacture include vials (including sealed vials).

The following examples are included for illustrative purposes and are not intend to limit the scope of the invention.

### EXAMPLES

### Example 1: Materials and Methods

Provided here are materials and methods used in the subsequent examples. In Vitro Methylation of H1.0 Protein and Peptides—Radiolabel In Vitro Methylation Assay

In order to visualize methylation of H1.0 peptide by G9A methyltransferase in vitro, a methylation assay was performed incorporating a radiolabelled methyl donor, which allows for visualization of methylated peptide on a gel after autoradiography exposure. The following methylation reaction was set up in a 1.5 ml tube: 1×HMT reaction buffer (50 mM Tris-HCl, 5 mM MgCl₂, 4 mM dithiothreitol, pH 9.0), 10 U G9A Methyltransferase (NEB, M0235S, Lot #0031201), 3.2 mM Adenosyl-L-Methionine, S-[Methyl-3H] (Perkin Elmer, NET155H, Lot #1664720), 10 μg H1.0 peptide (ThermoFisher Scientific, Biotin-AKPVKASKPKKAKPVKPK (SEQ ID NO:75)), final volume 10 μl. A control reaction as above but without H1.0 peptide was also created. Reactions were incubated in a thermocycler at 37° C. for 1 hour. Reactions were stopped by incubating on ice for 5 minutes, and subsequently resolved on a 16.5% Tricine gel (BioRad, 4563063). Gels were soaked in 30% methanol, 5% glycerol for 30 minutes before vacuum drying at RT for 24 hours. Autoradiography analysis of dried gels with a phosphorimager (Molecular Dynamics) was then performed to assess effective methylation of H1.0 peptide with methyl-3H groups by the G9A methyltransferase.

### Liquid Chromatography and High-Resolution Mass Spectrometry (LC-MS)

In order to identify the precise sites of methylation by G9A and GLP methyltransferases on histone H1.0, in vitro methylation assays were performed and subsequently analyzed by Liquid Chromatography and High-resolution Mass Spectrometry (LC-MS) analysis. Methylation reactions were set up using either G9A methyltransferase (NEB, M0235S, Lot #0031201) or GLP methyltransferase (Cayman Chemical, 10755). The methyl donor in each reaction was unlabeled S-Adenosyl-L-Methionine (NEB, B9003S). The H1.0 substrate in each reaction was either unmodified H1.0 peptide labeled with biotin (ThermoFisher Scientific, Biotin-AKPVKASKPKKAKPVKPK (SEQ ID NO:75)), di-methylated H1.0 peptide (ThermoFisher Scientific, Biotin-AKPVKASKPKKAKPVK^{(me2)}PK (SEQ ID NO:76)), or full-length recombinant human H1.0 (NEB, M2501S). Methylation reactions were set up in triplicate for each condition as follows: 1×HMT reaction buffer (50 mM Tris-HCl, 5 mM MgCl₂, 4 mM dithiothreitol, pH 9.0); 10 U G9A or GLP Methyltransferase; 3.2 mM S-Adenosyl-L-Methionine; 500 ng unmodified or modified H1.0 peptide, or 1 μg full-length protein; final volume 10 μl. As controls, reactions were set up as above but without any methyltransferase present. All reactions were incubated in a thermocycler at 37° C. for 1 hour, and subsequently stopped by incubating on ice for 5 minutes. Samples were frozen at −80° C. prior to LC-MS analysis.

For the Liquid Chromatography and High-resolution Mass Spectrometry (LC-MS) analysis, samples were prepared as described above and about 1 μg o the product was injected onto a Thermo Scientific Easy nLC system configured with a 10 cm×100 um trap column and a 25 cm×100 um ID resolving column. Buffer A was 98% water, 2% methanol, and 0.2% formic acid. Buffer B was 10% water, 10% isopropanol, 80% acetonitrile, and 0.2% formic acid. Samples were loaded at 4 uL/min for 10 min, and a gradient from 0-45% B at 375 nL/min was run over 130 min, for a total run time of 150 min (including regeneration and sample loading). The Thermo Scientific LTQ Orbitrap Velos mass spectrometer was run in a standard Top-10 data-dependent configuration except that a higher trigger-threshold (20K) was used to ensure that the MS2 did not interfere with the full-scan duty cycle. This ensured optimal full-scan data for quantification. MS2 fragmentation and analysis were performed in the ion trap mass analyzer. Samples were run in triplicate.

### LC-MS Data Analysis

Protein identification was performed using Thermo Scientific Proteome Discoverer version 1.4 (including Sequest and Percolator algorithms) using RefSeqHuman sequence database. These searches were performed with the control reactions lacking any methyltransferase enzyme. The Percolator peptide confidence filter was set to “high”. Protein quantification was performed using Pinpoint version 1.4 software. The Pinpoint quantification workflow included importing the Proteome Discoverer .msf files as spectral libraries. Identified peptides were subsequently quantified in MS .raw files using the Pinpoint peak finding, chromatographic alignment and area calculation algorithms.

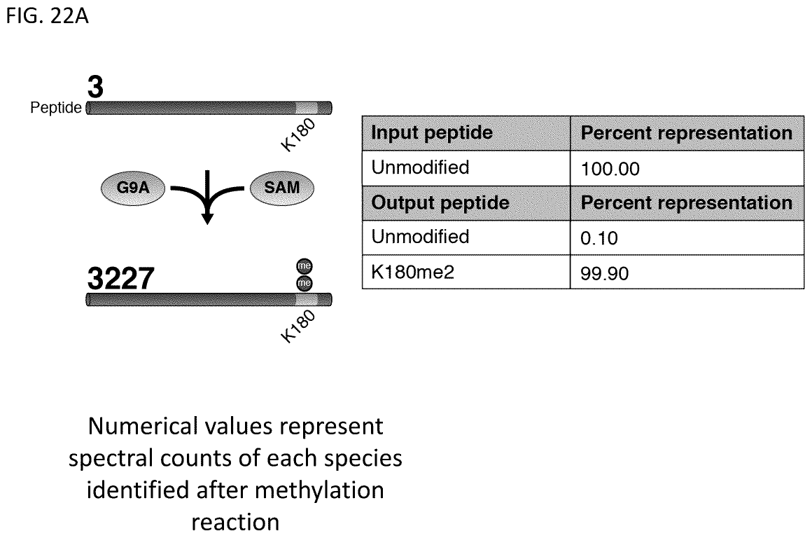

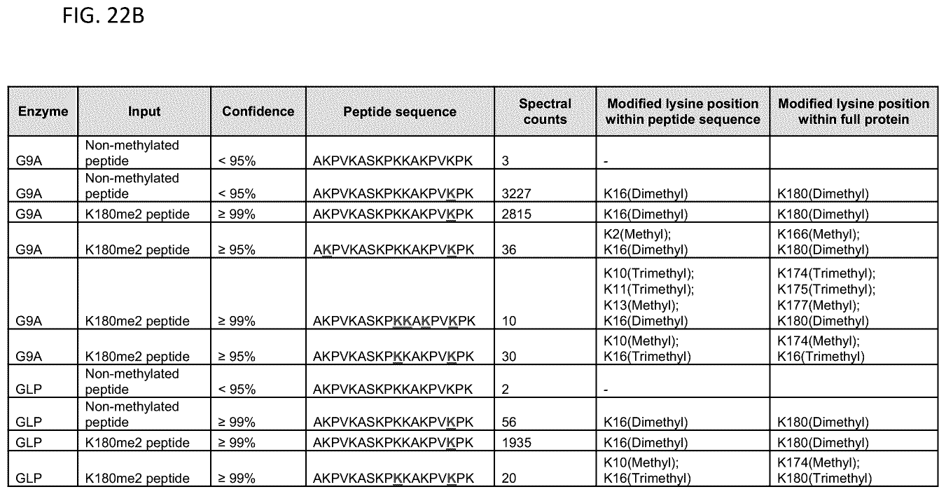

In order to identify the precise locations of G9A methylation on unmodified H1.0 peptide (AKPVKASKPKKAKPVKPK (SEQ ID NO:42)), a methylation reaction was set up and the products identified by LC-MS. The methylation reaction comprised recombinant G9A, an unlabeled methyl donor (S-Adenosyl-L-Methionine), and unmodified H1.0 peptide. The methylation reaction was subsequently analyzed by LC-MS, and each spectral peak (corresponding to a peptide species in the final reaction) was identified and quantified using spectral counts. The number of “me” circles in FIG. 22A represents the methylation state of the lysine residue (mono-, di-, or tri-methylated). FIG. 22A shows that in the presence of unmethylated H1.0 peptide, G9A specifically and abundantly dimethylates H1.0K180 (99.9% of all peptides).

Culture of hADSCs

Human adipose-derived mesenchymal stem cells (hADSCs) were obtained commercially from Life Technologies (R7788-115) and the American Type Culture Collection, ATCC (PCS-500-011). All cell lines were isolated from human adipose tissues obtained from three healthy adult female Caucasian donors aged 38, 45 and 49 undergoing routine liposuction procedures. Flow cytometry analysis and immunostaining analysis confirmed cells were positive for CD29, CD44, CD73, CD90, CD105, and CD166 and negative for CD14, CD31, CD34, and CD45. Cell lines were confirmed capable of adipogenic, chondrogenic and osteogenic differentiation under in vitro conditions.

Isolated adipose-derived stem cell lines were grown in DMEM/F12 medium (Life Technologies, 11330-057), supplemented with 10% (v/v) Fetal Bovine Serum (FBS) and 50 U/ml penicillin/streptomycin at 37° C./5% CO₂. Cumulative population doublings (PD) were calculated as PD=log(N/N0)×3.33 across the multiple passages as a function of the number of days of growth in culture, where N0 is the number of cells plated in the flask and N is the number of cells harvested at this passage. hADSCs PD 4-10 for self-renewing populations (SR) and PD 41-46 for replicatively senescent populations (REP-SEN) were used in all experiments.

### Senescence Induction and Assessment

For replicative senescence, hADSCs were grown in culture until reaching replicative exhaustion (PD 41-46). For acute DNA damage conditions, SR hADSCs (PD 4-10) were treated with 50 μg/ml bleomycin sulfate (Cayman Chemical, 13877) in growth media for 2 hours. For genotoxic stress-induced senescence, SR hADSCs (PD 4-10) were treated with 50 μg/ml bleomycin sulfate (Cayman Chemical, 13877) in growth media for 2 hours then washed with PBS and given fresh growth media without bleomycin. Cells were then grown for 3 days before collection.

To assess cellular senescence, cells were scored for senescence markers, including growth arrest, SA-β-gal activity, and the presence of persistent DNA-damage foci. The assay for monitoring the expression of pH-dependent senescence-associated β-galactosidase activity (SA-β-Gal) was performed as described in manufacturer's kit (BioVision). The cultured hADSCs were fixed with fixative solution for 15 minutes at room temperature, washed with twice with PBS and stained with X-Gal containing staining supplement overnight at 37° C. The cells were washed twice with PBS, and the images were captured using light microscopy (Leica, DMiL). DNA-damage foci were assessed by immunostaining for γH2A.X foci, as described below. γH2A.X foci appearance is indicative of double-strand breaks on DNA, or DNA damage. γH2A.X foci are well accepted molecular markers of double-strand breaks.

### Antibodies

Primary antibodies used: Anti-H1.0K180me2, 1:100 dilution, rabbit polyclonal, Aviva Systems Biology. Anti-H1.0 total, 1:500 dilution, EMD Millipore MABE446. Anti-γH2A.X (Ser139Ph), 1:500-1000 dilution, EMD Millipore 05-636. Anti-beta-actin, 1:2000, Abcam ab6276. Anti-poly-(ADP)-ribose, 1:500, Enzo Life Sciences ALX-804-220-R100. Anti-G9A, 1:500, Bethyl Laboratories A300-933A. Anti-GLP, 1:500, Bethyl Laboratories A301-643A. Anti-H3K9me2, 1:1000, Abcam A301-643A.

Secondary antibodies: Goat-anti-mouse-HRP, 1:4000, Biorad 1706516. Goat-anti-rabbit-HRP, 1:4000, Biorad 1706515. Goat-anti-human-HRP, 1:4000, Biorad 1721050, AlexaFluor-488-donkey anti-mouse, 1:5000, Life Technologies A-21202, AlexaFluor-488-donkey anti-rabbit, 1:5000, Life Technologies A-21206, AlexaFluor-555-donkey anti-mouse, 1:5000, Life Technologies A-31570, AlexaFluor-555-donkey anti-rabbit, 1:5000, Life Technologies A-31572, and anti-IgM HRP (Rabbit Anti-Human IgM (Mu chain) (HRP-Conjugate) (Abcam, ab97210, lot #GR169227-10)).

### ELISA

The following protocols describe a generalized enzyme-linked immunosorbent assays (ELISA) for the direct or indirect detection of serum H1.0K180me2 IgG levels.

FIG. 4 shows the use of sandwich ELISA for the indirect detection of serum H1.0K180me2 levels (for example for the indirect detection of IgG or IgM levels). The ELISA is used for the detection of antibodies to the H1.0K180me2 peptide epitope in samples of bodily fluids. The following protocol describes the generalized enzyme-linked immunosorbent assays (ELISA) using biotinylated peptides to capture specific antibodies in serum samples (for the indirect detection of serum H1.0K180me2 IgG or IgM levels), followed by detection with secondary antibodies conjugated to HRP, and tetramethylbenzidine (TMB) incubation. Wells of streptavidin-coated microplates (ThermoFisher, 15501) were washed 3× with wash buffer (Tris-buffered saline (25 mM Tris, 150 mM NaCl; pH 7.6) plus 0.1% BSA and 0.05% Tween-20). 50 pmols custom biotinylated H1.0K180me2 peptide (ThermoFisher) were diluted in wash buffer and bound to each well for 2 hours at RT with gentle shaking. Unbound peptide was washed away by 3× washes with wash buffer. Serial dilutions of H1.0K180me2 antibody in wash buffer were created as standards (1:25,000, 1:50,000, 1:100,000, 1:200,000, 1:400,000, stock concentration 0.54 mg/ml). Serum samples were diluted in wash buffer (1:1,000). 100 μL of standards and samples were added to wells in duplicate. Wells were incubated for 1 hour at RT with gentle shaking. Unbound standard or sample was washed away by 3× washes with wash buffer. Secondary antibodies (either anti-IgG secondary antibodies, or anti-IgM secondary antibodies) conjugated to HRP were diluted in wash buffer (1:1,000) (for serum samples, goat-anti-human-HRP, Biorad 1721050; for standards, goat-anti-rabbit-HRP, BioRad 1706515). 100 μL of secondary antibody dilutions were added to wells. Wells were incubated for 1 hour at RT with gentle shaking. Unbound secondary antibody was washed away by 3× washes with wash buffer. 100 μL of TMB solution (PeproTech) was added to each well and incubated for 15 minutes at RT with gentle shaking. 100 μL of stop solution (0.18 M H₂SO₄) was added to each well and incubated for 5 minutes at RT with gentle shaking. Upon oxidation, TMB forms a water soluble blue reaction product that may be measured spectrophotometrically at 650 nm. Upon acidification with stop solution, the reaction product becomes yellow with an absorbance peak at 450 nm. Absorbance of each well was measured at 450 nm with a plate reader (Molecular Devices). The concentration of H1.0K180me2 IgG or IgM in each sample was determined by extrapolation from a standard curve and described further below. FIG. 14C shows the use of an ELISA for the indirect detection of autoanti-H1.0K180me2 IgM levels in bodily fluids.

FIG. 5A shows the use of sandwich ELISA for the direct detection of H1.0K180me2 peptide epitope in samples of bodily fluids. Generally, an antibody specific to an H1.0K180me2 epitope is provided, and immobilized (coated) on a microplate. A clinical sample containing an H1.0K180me2 epitope for quantification is provided. The sample is added to the microplate, and the H1.0K180me2 epitope binds to the immobilized antibodies. Unbound materials are washed away. Detection antibodies are then added, for example an HRP or any other labelled antibodies. These detection antibodies bind the capture epitope. Unbound detection antibodies are washed away. A detection substrate solution is added, and a fluorophore or color change is measured. This is then quantified against a standard curve to report levels of H1.0K180me2 epitope in the clinical sample.

FIG. 5B shows the standard curve for H1.0K180me2 antigen sandwich ELISA test. In vitro methylated full-length H1.0K180me2 was used as a standard in a 2× dilution series from 50 ng/ml to 3.125 ng/ml. Standard concentration is plotted against measured O.D at 450 nm. A linear trend line was plotted between the points above the limit of detection, and the equation of the line was calculated (shown on plot). The insert shows the raw data. The ELISA was performed in duplicate and average O.D. values used for curve generation.

### Western Blot

Material for western blot analysis (cultured cells or tissue) was lysed in ice-cold RIPA lysis buffer (Thermo Scientific 89900) and sonicated using a Covaris S2 sonicator (10% duty cycle, Intensity 5, Bursts per minute 100, 120 seconds). Total protein concentration in each sample was quantified using Quick Start Bradford 1× Dye Reagent (BioRad, 5000205) following manufacturer's protocol. Samples were then mixed with NUPAGE LDS sample loading buffer (ThermoFisher, NP0007) and NUPAGE sample reducing buffer (ThermoFisher, NP0004), and heat denatured at 70° C. for 10 minutes. Proteins were separated on 4-12% precast polyacrylamide gels (ThermoFisher, NP0321) by electrophoresis and transferred to 0.45 μm nitrocellulose membrane. The membrane was blocked with 5% non-fat milk in PBS-T for 30 minutes at RT then immunoblotted with the above primary antibodies at 4° C. overnight. Proteins were detected with HRP secondary antibodies listed above for 1 hour at RT followed by ECL Western Blotting Substrate (ThermoFisher, 32106) using manufacturer's instructions. All washes between steps were with PBS-T. Membranes were imaged with Omega LUM-C imaging system (Gel Company).

### Immunofluorescence

For immunofluorescence, cells were cultured and treated in chamber slides, fixed in neutral 10% formalin, and permeabilized with PBS containing 0.5% Triton X-100. After washing, the slides were blocked using PBS containing 1% BSA and 4% donkey serum. After washing, the slides were incubated with primary antibodies listed above, washed again, incubated with AlexaFluor secondary antibodies listed above, and mounted with slow-fade gold (Molecular Probes) containing DAPI (to visualize nuclei). Cells were viewed by fluorescence microscopy and images were acquired for analysis using Spotfire software (Diagnostics Instruments).

### Slot Blot Analysis

0.5 μl of serum sample or known amounts of synthesized peptide were diluted in 200 μl TBS. Samples were then heat denatured at 70° C. for 10 minutes before being transferred to nitrocellulose membrane using a vacuum manifold slot blot apparatus (BioRad, 1706542). The membrane was blocked with 5% non-fat milk in PBS-T for 30 minutes at RT then immunoblotted with the above primary antibodies at 4° C. overnight. Proteins were detected with HRP secondary antibodies listed above for 1 hour at RT followed by ECL Western Blotting Substrate (ThermoFisher, 32106) using manufacturer's instructions. All washes between steps were with PBS-T. Membranes were imaged with Omega LUM-C imaging system (Gel Company). Slot blot bands for each sample were then quantified using ImageJ.

### RNA-Seq Analysis

Total RNA was isolated from self-replicating and replicatively senescent cell culture samples using TRIzol reagent (Invitrogen) according to the manufacturer's protocol. Samples from two different hADSC cell lines were combined together for the relevant conditions and RNA concentrations were measured with the Qubit 2.0 fluorometer using the RNA HS Assay kit (Invitrogen, Life technologies). The ERCC RNA Spike-In Control mix (Ambion, Life Technologies) was added to total RNA for quality control analysis. Subsequently, rRNA depletion was performed with the Low Input Ribominus Eukaryote System v2 (Ambion, Life technologies). cDNA libraries were constructed with Ion total RNA-seq kit v2 (Ambion, Life technologies), and barcoded with Ion Xpress RNA-seq barcode (Ambion, Life technologies). The size distribution and quantification of the libraries was performed with DNA HS bioanalyzer kit on a Bioanalyzer 2100 (Agilent Technologies). Library sequencing was performed on the Ion Proton System with P1 chip (Life Technologies), and each library was sequenced 3 times.

RNA-seq reads from individual Ion Proton System sequencing runs were combined for each library. Sequence reads were mapped to the reference human genome assembly hg19 (GRCh37) using the Torrent Mapping Alignment Program (TMAP, Life technologies). The quality of the RNA-seq runs for each condition was evaluated by comparing the expected counts of ERCC spike-in RNA sequences, obtained from the manufacturer's website, against the observed counts of RNA-seq tags that map to the same sequences. Initial gene expression levels were taken as the sum of exon-mapped reads for individual NCBI RefSeq gene models (c), and lowly expressed genes (read counts per million <1) were removed from subsequent analyses. For each library, individual gene expression levels were normalized using the beta-actin (ACTB) expression levels (cACTB) and the total exon length l of each gene. For library j, the beta-acting normalization factor sj was calculated as:

$${S}_{j} = \frac{\frac{1}{n} {\sum }_{k = 1}^{n} {c}_{ACT B , k}}{{c}_{ACT B , j}}$$

The final normalized expression value for gene i in library j was calculated as:

$${e}_{i , j} = \frac{{c}_{i , j} \times \text{?}}{{l}_{i}} \\ \text{?} \text{indicates text missing or illegible when filed}$$

### Drug Treatments

Bleomycin treatment: Cell growth media was supplemented with 50 g/ml bleomycin (Cayman Chemical 13877) for 2 hours to induce DNA double strand breaks. Cells were either collected immediately post-bleomycin treatment (acute DNA damage), or grown for 3 days post-bleomycin treatment (genotoxic stress induced senescence). PARP-1 Inhibitor: Cell growth media was supplemented with 1 μM of the potent PARP-1 inhibitor AG14361 (Selleckchem S2178) for 24 hours prior to downstream analysis. Rapamycin treatment: Cell growth media was supplemented with 500 nM Rapamycin (Cayman Chemical 11346) for 24 hours prior to downstream analysis or treatment. Everolimus treatment: Cell growth media was supplemented with 500 nM Everolimus (Cayman Chemical 11597) for 24 hours prior to downstream analysis or treatment. Temozolomide treatment: Cell growth media was supplemented with 50 μg/ml Temozolomide (Cayman Chemical 14163) for 2 hours prior to downstream analysis.

Analysis of H1.0K180Me2 in Mice after Irradiation

Blood was collected from two 13-month-old mice via cheek puncture and serum separated using Microtainer Tubes® with SST Serum Separator (BD, 365956). The same mice were then exposed to 7 Gy ionizing radiation (in the form of X-rays). Blood was drawn again from one mouse 2 hours after irradiation, and from the other mouse 48 hours after irradiation. Serum was again separated from blood using a Microtainer Tube®. Serum from each mouse before and after irradiation was then analyzed by slot blot and western blot.

Adipose Derived Stem Cell (ADSC) Isolation from Mice

Mouse adipose derived stem cells were isolated from wild type, mice. Subcutaneous or perineal white adipose tissue was collected and suspended in Hank's Buffered Salt Solution (HBSS), 3.5% Bovine Serum Albumin (BSA), 1% Collagenase, type II (Sigma) in 1:3 w/v ratio and shaken at 37° C. for 50 min. The cells were filtered through a 70 μm mesh cell strainer (BD Falcon #352350), treated with Red Blood Cell Lysis buffer (150 mM NH4Cl, 10 mM KHCO3, 0.1 mM EDTA, pH 7.3), and expanded ex-vivo in DMEM/F12 complete medium (DMEM/F12, 10% FBS, 100 U/ml penicillin, 100 μg/ml streptomycin, 2.5 μg/ml amphotericin B; Invitrogen) in 10% CO₂ at 37° C. and passaged at 80% confluency, changing medium every 72-96 hours. Cells were then used for western blot analysis as described elsewhere.

### Example 2: Discovery of H1.0K180Me2 Peptides in Senescence

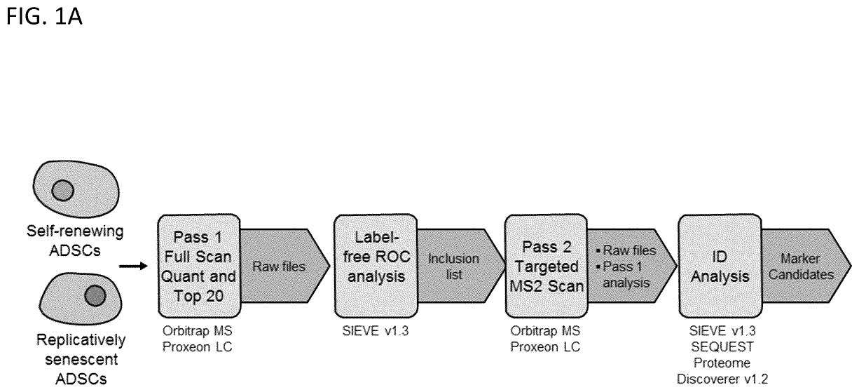

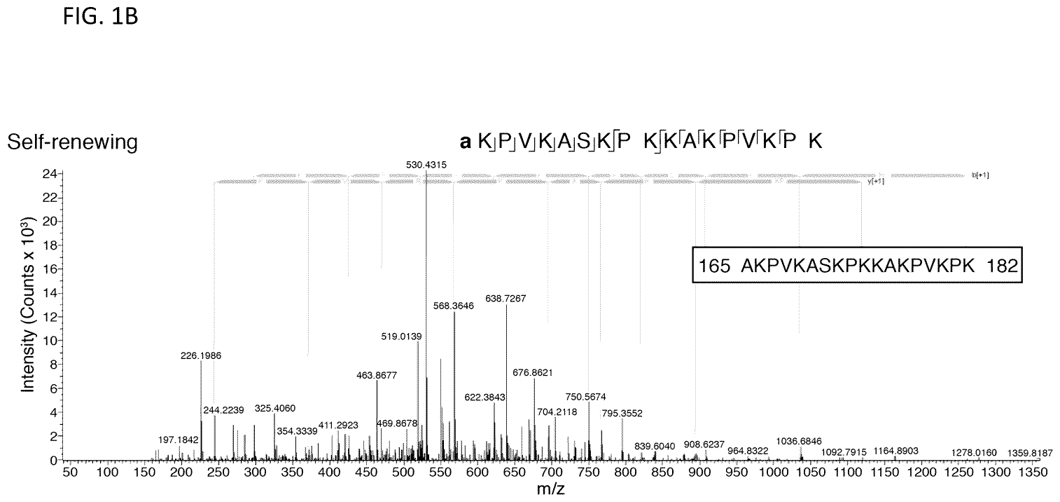

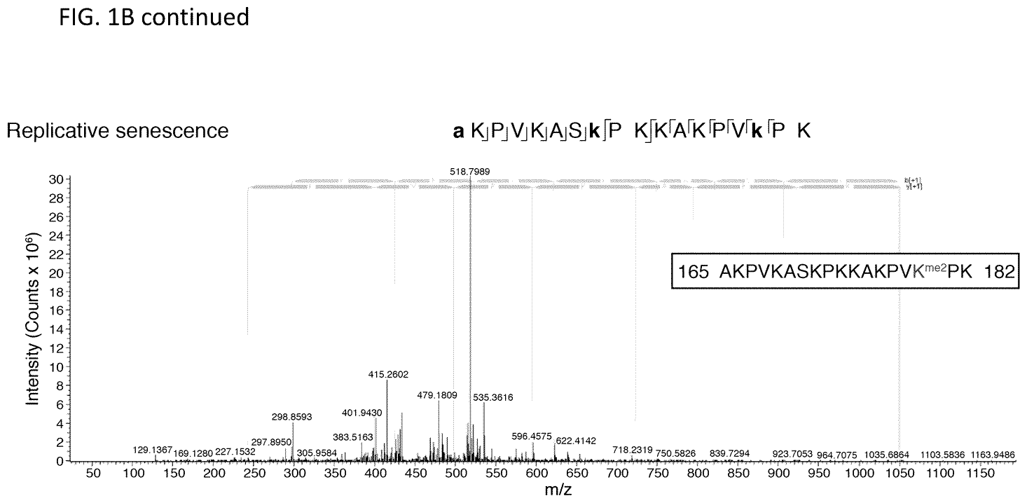

Self-renewing (SR) hADSCs and replicatively senescent (REP-SEN) hADSCs were obtained according to methods described in Example 1. To identify new aging-related post-translational modifications (PTMs) of histones, lysates from SR and REP-SEN hADSCs were assessed by M/Z Pair Tag LC-MS according to the discovery pipeline of FIG. 1A. Five technical replicate injections of self-renewing and replicatively senescent hADSC lysates were processed in a full-scan optimized configuration. It was determined that the chromatography and instrument methods for optimal full scan quantitative measurements conflicted with methods of optimal fragmentation scans. Therefore the mass spectrometer's accurate mass and broad dynamic range capabilities were exploited by incorporating into the analysis two distinct passes of data measurement. The first pass focused upon acquiring un-compromised and optimized full scan (MS) data for highly reproducible quantification. This first full-scan quantitative pass was used to generate an inclusion list of potentially interesting features. The inclusion list was then used for targeted fragmentation scan acquisition during a second pass for a subset of the data samples. As shown in FIG. 1B, the mass spectra revealed unmodified (AKPVKASKPKKAKPVKPK (SEQ ID NO:42)) peptides in SR hADSCs, and dimethylated (AKPVKASKPKKAKPVK^{me2}PK (SEQ ID NO:3)) peptides in REP-SEN hADSCs.

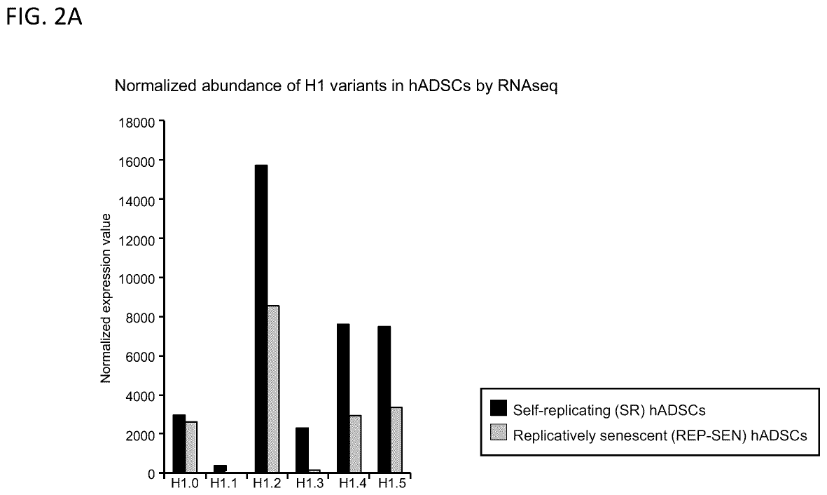

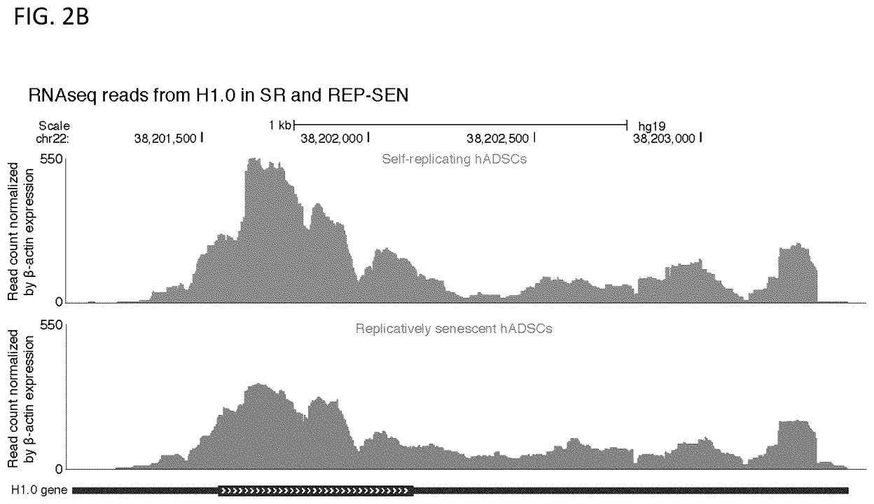

RNA-seq experiments comparing the expression profiles of SR and REP-SEN hADSCs were carried out according to methods described in Example 1. As shown in FIG. 2A, there are 6 distinct variants of histone H1: H1.0, H1.1, H1.2, H1.3, H1.4, and H1.5 in both SR (black bars) and REP-SEN (gray bars) hADSCs. Values on the y-axis represent the sum of exon-mapped reads for each gene, normalized by relative beta-actin expression in each sample, and further normalized by the total exon length for each gene. Histone H1.0 was found to be the 4^{th} most abundant variant of H1 at the gene expression level—H1.0 mRNA represented 8% of total H1 expression in SR hADSCs and 14% of total H1 expression in REP-SEN hADSCs. However unlike other variants, its expression remains constant during replicative senescence. Genome browser view (FIG. 2B) of all reads uniquely mapping to the histone H1.0 gene identified by RNA-seq analysis showed higher H1.0 expression in SR hADSCs (top track) than in REP-SEN hADSCs (bottom track). Read count was normalized by relative beta-actin expression in each sample.

The alignment of various peptide sequences centered around known methylated lysine residues revealed that amino acid sequence surrounding H1.0K180 is unique and not similar to other methylated lysine residues (FIG. 3A).

### Example 3: H1.0K180Me2 Antibody

A polyclonal antibody specific to the H1.0K180me2 antigen was made as follows: Two New Zealand rabbits were immunized for antibody production. Injections were administered subcutaneously (SQ) as emulsions of Keyhole Limpet Hemocyanin (KLH) conjugated to an H1.0K180me2 peptide (Peptide: CAKPVKASKPKKAKPVK(me2)PK (SEQ ID NO:39); Conjugated Peptide: (KLH-CAKPVKASKPKKAKPVK(me2)PK (SEQ ID NO:77)), where the C-terminal C is artificially added to the sequence of a peptide to generate the covalent link to KLH, in Complete Freund's Adjuvant (CFA) or Incomplete Freund's Adjuvant (IFA). Initial immunization was performed with 0.5 mg of antigen in CFA at 10 SQ sites. 6 subsequent booster immunizations were performed at routine intervals over a 7-8 week period. Boosters consisted of 0.25 mg antigen in IFA at 4 SQ sites. Rabbits were bled at week 5 (˜25 ml blood per rabbit) and at week 8 (˜50 ml blood per rabbit). The titer of the immunization was assessed using ELISA by comparing pre- and post-immunization bleeds. The antibody was then purified using antigen affinity chromatography. The antigen used for purification was either biotin- or bovine serum albumin (BSA)-conjugated H1.0K180me2 peptide (biotin-CAKPVKASKPKKAKPVK(me2)PK (SEQ ID NO:78)) or BSA-CAKPVKASKPKKAKPVK(me2)PK (SEQ ID NO:79)). The peptides were dimethylated using the in vitro methylation methods provided herein.

The H1.0K180me2 antibody was tested as follows. To check the specificity of H1.0K180me2 antibody, histone H1.0 peptides methylated on K172, K174, K175, K177, or K180 were transferred to a membrane using a vacuum-manifold slot blot in a dilution series. The membrane was then immunoblotted using the H1.0K180me2 antibody to identify potential cross-reactivity with other methyl-lysine groups. The H1.0K180me2 antibody is highly specific for H1.0K180me2, even in low concentrations (FIG. 3B). To further check the specificity of H1.0K180me2 antibody, histone H3 peptides methylated on K4, K9, K27, K36 or K79 were transferred to a membrane using a vacuum-manifold slot blot in a dilution series. The membrane was then immunoblotted using the H1.0K180me2 antibody to identify potential cross-reactivity with other methyl-lysine groups. This antibody exhibited no cross-reactivity with any methylated H3 peptides analyzed (FIG. 3C). The slot blot analysis protocol was carried out according to methods described in Example 1.

The specificity of the rabbit anti-H1.0K180me2 IgG antibody was determined using the anti-H1.0K180me2 IgG ELISA test. Wells were coated with either H1.0K180me2 peptide, H1.0K180me1 peptide, or unmodified peptide, in a 2× dilution series from 6.25 pmol to 781 fmol. 6.67 fmol of rabbit anti-H1.0K180me2 IgG antibody in 100 ul buffer were added to each well and antibody binding efficiency was monitored by ELISA. Curves for each peptide were generated by plotting peptide amount against measured O.D at 450 nm. Raw ELISA data is given in the table the figure for the two experimental repeats. (FIG. 3D)

The rabbit anti-H1.0K180me2 IgG antibody is 2.7×more efficient at binding H1.0K180me2 peptide than H1.0K180me1 peptide. Within the optimal linear range, 1 molecule of rabbit anti-H1.0K180me2 IgG antibody recognizes 1 out of 117 molecules of H1.0K180me2 peptide, but only 1 out of 316 molecules of H1.0K180me1 peptide. Unmodified peptide is not recognized in this range.

ClustalW2 alignment of histone H1 variant protein sequences revealed that H1.0K180 was embedded in a unique sequence of amino acids not present in other H1 variants (FIG. 3E). The peptide identified in the initial discovery experiment is highlighted in gray. The globular domain region comprising 3 α-helices is also shown.

### Example 4: Indirect Detection of Serum H1.0K180Me2 IgG and H1.0K180Me2 IgM Autoantibody Levels Using H1.0K180Me2 Peptides in an ELISA

### Indirect Detection of Serum H1.0K180Me2 IgG Autoantibodies

The following example describes an enzyme-linked immunosorbent assay (ELISA) using biotinylated therapeutic H1.0K180me2 peptides to capture specific autoantibodies in serum samples, followed by detection with secondary antibodies conjugated to HRP, and tetramethylbenzidine (TMB) incubation. Wells of streptavidin-coated microplates (ThermoFisher, 15501) were washed 3× with wash buffer (Tris-buffered saline (25 mM Tris, 150 mM NaCl; pH 7.6) plus 0.1% BSA and 0.05% Tween-20). 50 pmols custom biotinylated H1.0K180me2 peptide (ThermoFisher) were diluted in wash buffer and bound to each well for 2 hours at RT with gentle shaking. Unbound peptide was washed away by 3× washes with wash buffer. Serial dilutions of the H1.0K180me2 antibody in wash buffer were created as standards (1:25000, 1:50000, 1:100000, 1:200000, 1:400000, stock concentration 0.54 mg/ml). Serum samples were diluted in wash buffer (1:1000). 100 μL of standards and samples were added to wells in duplicate. Wells were incubated for 1 hour at RT with gentle shaking. Unbound standard or sample was washed away by 3× washes with wash buffer. Secondary antibodies conjugated to HRP (secondary labeled antibody, as depicted in FIG. 4, step 3) were diluted in wash buffer (1:1000) (for serum samples, goat-anti-human-HRP, Biorad 1721050; for standards, goat-anti-rabbit-HRP, Biorad 1706515). 100 μL of secondary antibody dilutions were added to wells. Wells were incubated for 1 hour at RT with gentle shaking. Unbound secondary antibody was washed away by 3× washes with wash buffer. 100 μL of TMB solution (PeproTech) was added to each well and incubated for 15 minutes at RT with gentle shaking. 100 μL of stop solution (0.18 M H₂SO₄) was added to each well and incubated for 5 minutes at RT with gentle shaking. Upon oxidation, TMB forms a water soluble blue reaction product that may be measured spectrophotometrically at 650 nm. Upon acidification with stop solution, the reaction product becomes yellow with an absorbance peak at 450 nm. Absorbance of each well was measured at 450 nm with a plate reader (Molecular Devices). The concentration of H1.0K180me2 IgG in each sample was determined by extrapolation from a standard curve.

The indirect detection of serum H1.0K180me2 IgG levels using ELISA is generally depicted in FIG. 4.

### Indirect Detection of Serum H1.0K180me2 IgM Autoantibodies

Detection of serum H1.0K180me2 IgM autoantibodies involved three steps. The test comprises of measurement of total IgM in patient serum using a commercially available sandwich ELISA kit (Step 1), followed by measurement of anti-H1.0K180me2 IgM in the same sample using an indirect ELISA assay (Step 2). As the final step, anti-H1.0K180me2 IgM levels were normalized by total IgM in the sample (Step 3).

Step 1: The total human IgM in the patient serum was measured using a commercially available sandwich ELISA kit purchased from Affymetrix eBioscience (Human IgM ELISA Ready-SET-Go!, part #88-50620, lot #123620111).

Step 2: Measurement of anti-H1.0K180me2 IgM in patient serum using a customized indirect ELISA assay. Biotin-labeled H1.0K180me2 capture peptides were coated onto a 96-well streptavidin-coated microplate (ThermoFisher, 15501, lot #QH218700). Excess free peptide was washed away with wash buffer (Tris-buffered saline (25 mM Tris, 150 mM NaCl; pH 7.6) plus 0.1% BSA and 0.05% Tween-20). Non-specific binding sites on the microplate were blocked using blocking buffer (Thermo Scientific, 37536, lot #QJ222191) containing d-biotin (VWR, 97061-444, lot #1405C080; to block unbound streptavidin on the plate surface), which reduced background noise. Diluted patient serum was added to microplate wells in triplicate. Anti-H1.0K180me2 autoantibodies in the patient sample recognized and bound the capture peptide on the plate surface. In parallel, a dilution series of anti-H1.0K180me2 IgG antibody standard was processed on the same plate. The wells were then washed with wash buffer to remove unbound sample or standard. Bound anti-H1.0K180me2 IgM autoantibodies were then detected using an anti-IgM HRP detection antibody (secondary labeled antibody, as depicted in FIG. 4, step 3; Rabbit Anti-Human IgM (Mu chain) (HRP-Conjugate) (Abcam, ab97210, lot #GR169227-10)) added to each sample well. The standards were detected using an anti-IgG HRP detection antibody (Goat Anti-Rabbit IgG (H+L) (HRP-Conjugate) (BioRad, 1706515, lot #350003080)) added to each standard well. Excess unbound detection antibody was then washed off with wash buffer. The bound HRP activity was then determined by the addition of tetramethylbenzidine (TMB) substrate (KPL, 52-00-01, lot #10164336) to the wells. This reaction was halted with the addition of Stop Solution ((0.18 M H₂SO₄) (KPL, 50-85-04, lot #10164328)) and the absorbance of the contents of the wells was read at 450 nm. The absorbance is directly proportional to the concentration of anti-H1.0K180me2 IgM in the sample. The molar concentration of anti-H1.0K180me2 IgM in each sample was then determined by extrapolation from standard curve and multiplication by initial dilution factor.

The standard curve utilized the anti-H1.0K180me2 rabbit polyclonal IgG antibody. This antibody was confirmed to be highly specific for H1.0K180me2. This antibody does not recognize any other lysine methylation on H1.0 and is specific to degree of methylation, e.g. can discriminate between mono (me1), di (me2) and tri (me3) of lysine K180 (FIG. 3A). It does not cross-react with methylation sites on other common histone proteins (FIG. 3A). The standard curve was generated from a dilution series of this anti-H1.0K180me2 IgG (72, 36, 18, 12, 9, 4.5 fmol/ml) or a dilution series of total IgM antibodies (556, 444, 222, 111, 56, 28, 18 or 0 fmol/ml) with a fixed concentration of biotinylated epitope (synthetic peptide AKPVKASKPKKAKPVK^{me2}PK (SEQ ID NO:3)) or fixed amount secondary anti-IgM antibodies respectively. The molar concentration of anti-H1.0K180me2 IgM in each sample was inferred from the curve of anti-H1.0K180me2 IgG molar concentration.

Step 3: Normalization of anti-H1.0K180me2 IgM serum concentration by total IgM serum concentration. Measurement of total IgM in patient serum was performed in triplicate. The average total IgM concentration in each sample was then calculated from the triplicate repeats. Measurement of anti-H1.0K180me2 IgM in patient serum was performed in triplicate. The average anti-H1.0K180me2 IgM concentration in each sample was then calculated from the triplicate repeats. To obtain the final test value, the average anti-H1.0K180me2 IgM concentration was divided by the average total IgM concentration for each patient sample.

$$Test value = \frac{Anti - H 1. K 180 me 2 concentration}{Total IgM concentration}$$

The indirect detection of serum H1.0K180me2 IgM levels using ELISA is generally depicted in FIG. 4.

### Measurement of Anti-H1.0 (Unmodified H1.0) IgM Levels in Patient Serum

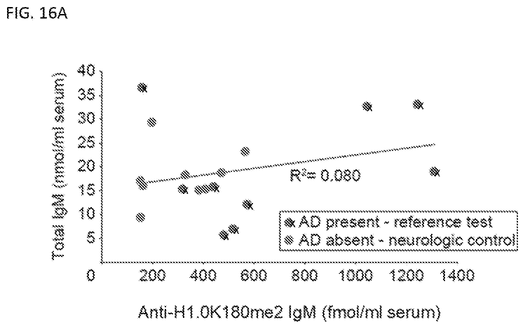

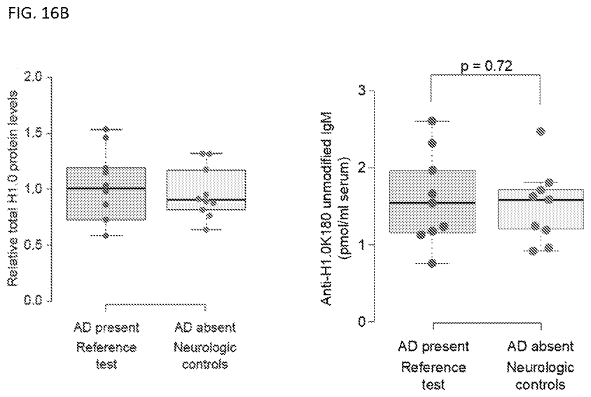

The procedure was identical to the indirect ELISA for anti-H1.0K180me2 IgM, however the capture peptide was different. In this case the peptide was an unmodified peptide lacking K180me2 (unmodified H1.0). Also, the standard used was a rabbit polyclonal IgG raised against unmodified peptide. (FIG. 16B, right panel).

### Example 5: Use of an H1.0K180Me2 Antibody Shows that H1.0K180Me2 is Associated with Acute DNA Damage

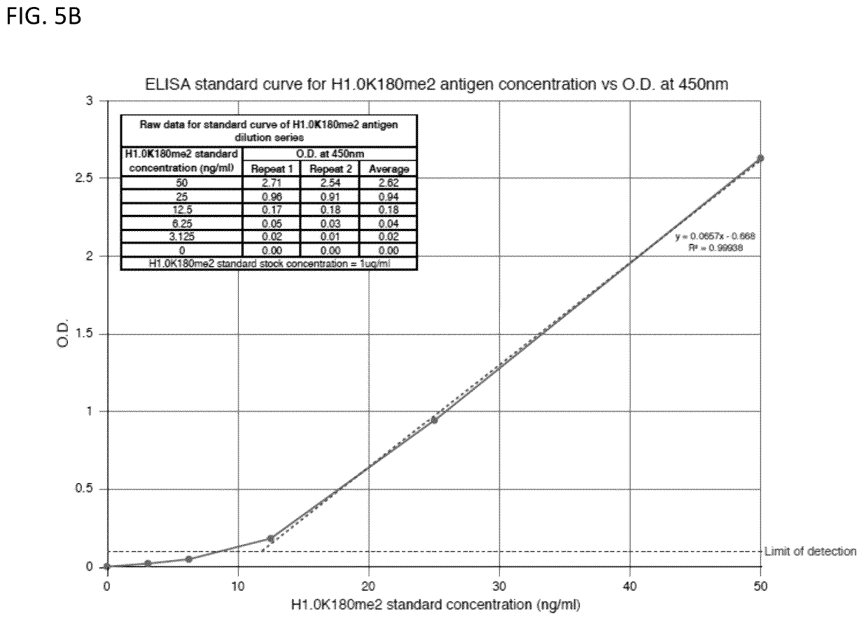

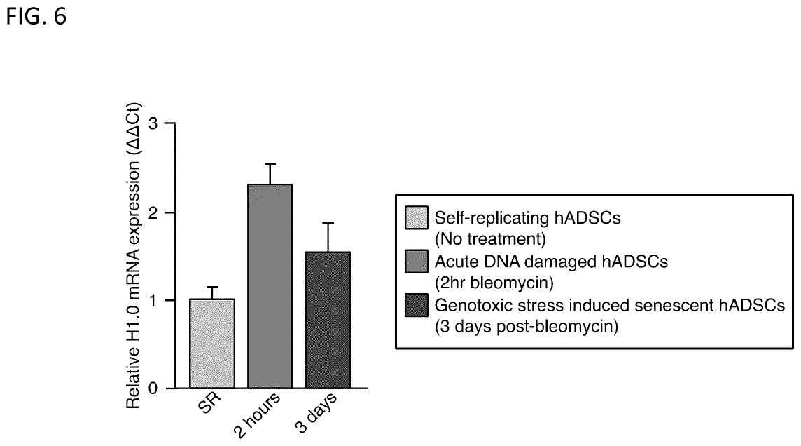

FIG. 6 shows that H1.0 mRNA expression increases upon acute DNA damage and genotoxic stress-induced senescence, according to methods described in Example 1. RT-PCR analysis of histone H1.0 mRNA expression was performed on total RNA derived from self-replicating hADSCs, hADSCs treated with bleomycin for 2 hours (acute DNA damage) and hADSCs treated with bleomycin and allowed to senesce for 3 days (genotoxic stress-induced senescence). H1.0 mRNA expression increases over 2-fold upon acute DNA damage, and remains elevated (1.5-fold over SR) upon genotoxic stress induced senescence.

To assess the relationship between H1.0K180 methylation and DNA damage, SR hADSCs and hADSCs treated with DNA damaging agent bleomycin for 2 hours (according to methods described in Example 1) were lysed and fractionated to obtain the chromatin bound fraction. Western blot analysis with a α-H1.0K180me2 antibody (performed according to methods described in Example 1) shows methylation of H1.0K180 on the chromatin upon DNA damage (FIG. 7A). Upon treatment of SR hADSCs with bleomycin to induce DNA double-strand breaks (according to methods described in Example 1), H1.0K180me2 localized to the cytoplasm.

By slot blot immunoassay, it was found that the presence of H1.0K180me2 was detected in the conditioned medium of hADSCs in a time course dependent manner after genotoxic insult imposed by bleomycin, with maximal H1.0K180me2 secretion detected within 48 hours (FIG. 7B). This signifies secretion out of the cells. An increase in DNA fragmentation factor (DFFB/DFF40/CAD) secretion upon GSI-SEN was seen, in a similar manner to H1.0K180me2 starting 48 hours after treatment (FIG. 7B). None of the protein is secreted in measurable quantities upon onset of acute DNA damage (ADD).

### Example 6: H1.0K180 Methylation is Independent of and Occurs Upstream of DNA Damage Repair Pathway Protein PARP-1 Activity

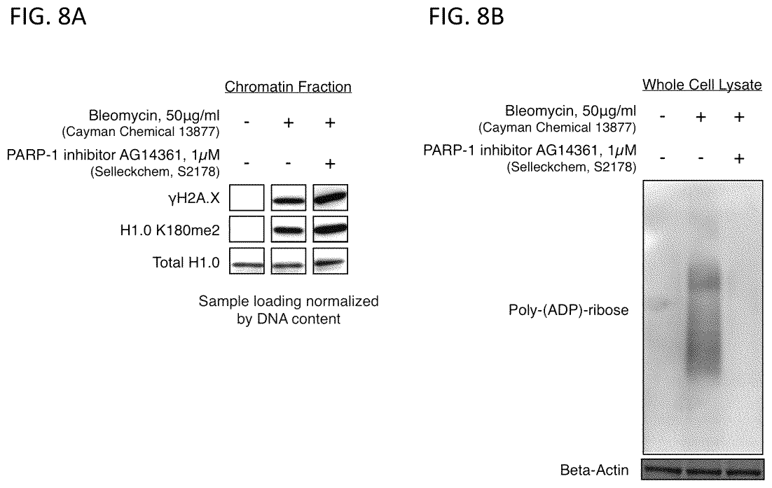

To further assess if H1.0K180 methylation is dependent on the DNA damage repair pathway protein PARP-1 activity, western blot analysis was performed according to methods described in Example 1, to examine chromatin-bound H1.0K180me2 and γH2A.X upon treatment with bleomycin. PARP-1, an early player in the DNA-damage response pathway, was inhibited using the potent inhibitor AG14361 according to methods described in Example 1. As shown in FIG. 8A, H1.0K180me2 appeared on chromatin upon DNA damage, and its appearance was independent of PARP-1 activity. In fact, PARP-1 inhibition led to an increased accumulation of H1.0K180me2 and γH2A.X (FIG. 8A). Western blot analysis of PARP-1 inhibitor activity in whole cell lysates revealed that, upon bleomycin treatment, PARP-1 activity led to increased poly-(ADP)-ribose (PAR) levels within the cell, which was abolished by PARP-1 inhibitor, suggesting efficient PARP-1 inhibition (FIG. 8B). Thus it was determined that H1.0K180 methylation is independent of DNA damage repair pathway protein PARP-1 activity

### Example 7: Use of an H1.0K180Me2 Antibody Shows that H1.0K180Me2 is Released from Chromatin Upon Genotoxic Stress-Induced Senescence and is Secreted into the Extracellular Matrix

SR hADSCs, hADSCs treated with bleomycin for 2 hours (acute DNA damage) and hADSCs treated with bleomycin and allowed to senesce for 3 days (genotoxic stress induced senescence) were lysed and fractionated into soluble and chromatin bound fractions. These fractions were then subjected to LC-MS/MS analysis. Peptide expression levels were taken as the total area under the LC-MS/MS relative intensity curve, and individual peptides were unambiguously assigned to proteins using Pinpoint software, version 1.4 (Thermo Scientific). The areas of all peptides assigned to an individual protein were summed to yield protein expression levels, which were normalized against the total protein library size for each sample. For each cellular fraction, H1.0 peptide abundance is represented as a percentage of total H1.0 peptides observed in each condition. Normalized absolute values of H1.0 peptide levels in each fraction were also shown. As shown in FIG. 9A, Chromatin bound H1.0 decreased from ˜60% of total in SR and acute DNA damage, to ˜30% of total upon genotoxic stress induced senescence. Culture media from SR hADSCs treated with bleomycin was collected for slot blot analysis with α-H1.0 and α-H1.0K180me2 antibodies to assess secretion of H1.0 to the extracellular matrix (ECM). The experiment was carried out according to methods described in Example 1. Secreted H1.0 was detectable in the cell culture media, and after 24 hours of bleomycin treatment, secreted H1.0K180me2 was also readily detected (FIG. 9B). These results confirmed that methylated H1.0K180 is released from chromatin upon genotoxic stress induced senescence and is secreted into the ECM.

### Example 8: Use of an H1.0K180Me2 Antibody Shows that Exposure to Ionizing Radiation Induces Increased Levels of H1.0k180Me2 in Serum

The effect of ionizing radiation on H1.0K180me2 levels in serum was examined. Serum was collected from wild-type mice before and either 2 hours or 48 hours after exposure to 7 Gy of ionizing radiation, according to methods described in Example 1. H1.0K180me2 serum levels 2 hours (mouse 1) or 48 hours (mouse 2) after irradiation were compared to initial levels before treatment using slot blot and immunoblotting with α-H1.0K180me2 antibody (FIG. 10A). The concentration of H1.0K180me2 in each serum sample was calculated using a standard curve of H1.0K180me2 peptide included in each analysis. Mouse serum albumin was used as a loading control. H1.0K180me2 dot blot bands were quantified and normalized by serum albumin. Relative increases in H1.0K180me2 after irradiation are shown (FIG. 10B). Western blot analysis of equal volumes of mouse serum with α-H1.0K180me2 antibody also showed increased H1.0K180me2 after irradiation (FIG. 10C).

### Example 9: Use of an H1.0K180Me2 Antibody and Labeled H1.0K180Me2 Peptides Show Shows that H1.0K180Me2 Level in Brain and Serum is an Indication of Alzheimer's Disease

Whole cell lysates of mouse brain samples from 1 month (young) and 24 month (old) mice were compared by western blot analysis with α-H1.0K180me2, α-H1.0, α-γH2A.X and α-β-Actin antibodies, according to methods described in Example 1. H1.0K180me2 levels increased in the 24 month mouse, correlating with increased levels of γH2A.X (FIG. 11A).

Further studies were carried out on human clinical samples.

Human sera were obtained from the Cooperative Human Tissue Network (CHTN) and Nuclea Biotechnologies (NCB). Sera were derived from three groups: 1) middle-aged healthy donors with no significant medical condition (n=7, age range=32-38 years old) 2) old healthy donors with no significant medical condition (n=9, age range=63-76 years old) 3) old donors with clinically diagnosed Alzheimer's disease (n=10, age range=75-103 years old). Further details of each donor in Table 4.

TABLE 4
Characteristics of Tissue Donors

| Sample Type | Age | Gender | Race | Patient Diagnosis |
|---|---|---|---|---|
| Brain | 23 | Female | White | Normal |
| Brain | 60 | Female | White | Normal |
| Brain | 73 | Female | White | Normal |
| Brain | 77 | Female | White | Normal |
| Serum | 32 | Female | White | Normal |
| Serum | 32 | Female | Black | Normal |
| Serum | 32 | Male | Black | Normal |
| Serum | 36 | Female | White | Normal |
| Serum | 36 | Male | White | Normal |
| Serum | 37 | Male | Black | Normal |
| Serum | 38 | Male | White | Normal |
| Serum | 63 | Female | White | Normal |
| Serum | 66 | Female | White | Normal |
| Serum | 67 | Female | White | Normal |
| Serum | 70 | Female | White | Normal |
| Serum | 70 | Female | White | Normal |
| Serum | 70 | Female | White | Normal |
| Serum | 73 | Female | Black | Normal |
| Serum | 74 | Female | White | Normal |
| Serum | 76 | Female | White | Normal |
| Serum | 83 | Male | Black | Alzheimer's disease |
| Serum | 103 | Male | White | Alzheimer's disease |
| Serum | 94 | Female | White | Alzheimer's disease |
| Serum | 77 | Male | Black | Alzheimer's disease |
| Serum | 78 | Female | Black | Alzheimer's disease |
| Serum | 78 | Male | White | Alzheimer's disease |
| Serum | 75 | Female | White | Alzheimer's disease |
| Serum | 91 | Female | Black | Alzheimer's disease |
| Serum | 93 | Female | Black | Alzheimer's disease |
| Serum | 90 | Female | Black | Alzheimer's disease |

Whole cell lysates of human brain samples from 23 years (young) and or greater than 60 years (old) healthy individuals were also analyzed by western blot with antibodies and methods described above. Table 4 provides characteristics of brain tissue donors. As shown in FIG. 11B, older individuals exhibited increased expression of H1.0K180me2. These results indicated increases of H1.0K180me2 with organismal age.

An objective of this study was to demonstrate utility of measurements of serum H1.0K180me2 and/or measurement of serum antibodies to H1.0K180me2 as biomarkers of Alzheimer's disease predictive performance. To ensure correct predictive capacity of the new diagnostic biomarker, the patient stratification method was developed and tested for its diagnostic accuracy. The performance of the test was evaluated by comparing it to the reference standard on record (for example a combination of several tests recommended by National Institute of Aging—Alzheimer's Association (NIA-AA) for the definitive diagnosis of AD. This study was designed and conducted according to Standards for Reporting of Diagnostic Accuracy (STARD). The statistical analysis included diagnostic accuracy parameters, cross-validation, and Youden Index optimization. According to the patient stratification strategy, and with the predictive power of the analysis, patients may be stratified as a category of patients with likelihood of Alzheimer's disease development. The sensitivity, specificity, positive and negative predictive values (PPV and NPV) as well as positive and negative likelihood ratios (PLR and NLR) are shown for each test design. In FIGS. 12A-12C, FIG. 13B, FIG. 14B, FIG. 14E, FIG. 15A and FIG. 15B, box plot center lines show the medians; box limits indicate the 25th and 75th percentiles as determined by R software; whiskers extend 1.5 times the interquartile range from the 25th and 75th percentiles, outliers are represented by dots; data points are plotted as open circles. The dashed line demonstrates the threshold calculated by ROC curve (Receiver Operating Characteristic Curve) analysis. The sensitivity, specificity, positive and negative predictive values (PPV and NPV) as well as positive and negative likelihood ratios (PLR and NLR)) are shown for each test design. The statistical methods help to predict the presence or absence of disease in the patients. The degree to which a test result modifies pre-test probability of disease is expressed by the “likelihood ratio” based on Bayes Theorem. Positive likelihood ratios (PLR) tells how much to increase the probability of the disease, if the test is positive. Negative likelihood ratio (NLR) tells how much to decrease the probability of the disease, if the test is negative. A PLR>1 Indicates an increase probability that the target disorder is present, a PLR<1 indicates a decreased probability that the target disorder is present, and a PLR=1 means that test does not change the probability of the disease. The ROC curve, thresholds and areas under the curve (AUC) are shown for each of the test's designs. The values of 0 in the 2×2 matrix (TP, FN, FP, TN) can make calculating ratios impossible. The “pseudocounts” of 0.5 was added to control for this. The “pseudocounts” are added to every value so they do not affect the relative values. Box plot center lines in FIGS. 12A-12C, FIG. 13B, FIG. 14B, FIG. 14E, FIG. 15A and FIG. 15B show the medians; box limits indicate the 25th and 75th percentiles as determined by R software; whiskers extend 1.5 times the interquartile range from the 25th and 75th percentiles, outliers are represented by dots; data points are plotted as open circles.

To test if H1.0K180me2 levels can serve as diagnostic indicator of Alzheimer's disease, H1.0K180me2 levels in human serum were quantified by slot blot analysis using α-H1.0K180me2 antibody, according to methods described in Example 1. Equal volumes of serum from healthy individuals of 30-40 years (n=7) or >60 years (n=9), and individuals with clinically diagnosed Alzheimer's disease of >60 years (n=10) were analyzed (Table 4 provides characteristics of serum donors).

### H1.0K180Me2 Levels

FIGS. 12A-C show that detection of the H1.0K180me2 antigen (FIGS. 12A, 12B, 12C), the detection of naturally occurring H1.0K180me2 IgG autoantibodies (FIG. 13B), and the detection of naturally occurring H1.0K180me2 IgM autoantibodies (FIGS. 14A-E; FIGS. 15A-B) in human serum may act as tools for early diagnostics of Alzheimer's disease.

FIG. 12A shows the quantification of H1.0K180me2 levels determined by slot blot analysis in Alzheimer's disease patients and age-matched controls. Quantification of H1.0K180me2 levels was determined by slot blot analysis in Alzheimer's disease patients and age-matched controls. The concentration of H1.0K180me2 in each serum sample was calculated using a standard curve of H1.0K180me2 peptide included in each analysis. As shown in FIG. 12A, Alzheimer's disease patients displayed lower concentrations of serum H1.0K180me2 than healthy age-matched controls, indicating that H1.0K180me2 serum concentrations may effectively segregate patients with Alzheimer's disease from healthy individuals and could act as a diagnostic tool for Alzheimer's disease detection. As shown in Table 5, measurement of H1.0K180me2 antigen concentration in serum at or below 5.61 nmol/ml is indicative of the disease presence with likelihood of 24%, compared to pre-test probability; and the positive likelihood ratio (PLR or LR+) is 3.6. Post-test probability is calculated based on following formula: Pre-test Odds×LR/(1+Pre-test Odds×LR), where pre-test odds are the clinical suspicion of disease present before testing. This is usually calculated from the Likelihood Ratio Nomogram or Fagan Nomogram (NEJM 1975; 293:257).

TABLE 5
H1.0K180me2 antigen concentration normalized by serum volume

| | | 95% |
|---|---|---|
| Metric | Value | confidence interval |
| Threshold | 5.61 | |
| Sensitivity | 80% | [56%, 100%] |
| Specificity | 78% | [40%, 100%] |
| PPV | 80% | [69%, 100%] |
| NPV | 78% | [60%, 100%] |
| LR+ | 3.60 | [2.03, 17.27] |
| LR− | 0.26 | [0.00, 0.60] |
| ln(OR) | 2.64 | [0.43, 4.85] |

FIG. 12B shows H1.0K180me2 levels in human serum normalized by total IgG serum levels. To normalize H1.0K180me2 levels in human serum by total IgG serum levels, total IgG levels were determined by slot blot analysis for each serum sample using goat anti-human IgG secondary antibody. H1.0K180me2 concentrations were normalized by observed IgG levels in each sample. As shown in FIG. 12B, H1.0K180me2 levels elevated in healthy >60 year individuals relative to healthy younger individuals (30-40 years), while patients with Alzheimer's disease exhibit significantly lower normalized levels of H1.0K180me2 relative to healthy aged individuals (>60 years). The left part of box graph are patients without prodromal Alzheimer's disease diagnosis or Mild Cognitive Impairment (MCI). The right part of box graph shows patients with post-mortem confirmation of Alzheimer's disease pathology. Table 6 shows the values from the ROC analysis.

TABLE 6
H1.0K180me2 antigen concentration normalized
by total IgG concentration

| | | 95% |
|---|---|---|
| Metric | Value | confidence interval |
| Threshold | 0.98 | |
| Sensitivity | 90% | [67%, 100%] |
| Specificity | 89% | [70%, 100%] |
| PPV | 90% | [73%, 100%] |
| NPV | 89% | [70%, 100%] |
| LR+ | 8.10 | [2.40, 19.09] |
| LR− | 0.11 | [0.00, 0.39] |
| ln(OR) | 4.28 | [1.35, 7.21] |

FIG. 12C shows H1.0K180me2 levels in human serum normalized by total protein levels. To normalize H1.0K180me2 levels in human serum by total protein levels, total serum protein was measured for each sample using a Qubit (Invitrogen). H1.0K180me2 concentrations were then normalized by measured protein concentration in each sample. As shown in FIG. 12C, H1.0K180me2 levels elevated in healthy >60 year old individuals, while patients with Alzheimer's disease exhibited significantly lower normalized levels of H1.0K180me2 relative to healthy aged individuals (>60 years). FIG. 12C shows the serological quantification of H1.0K180me2 antigen relative to total protein composition of serum. Table 7 shows the ratio of H1.0K180me2 antigen to total protein in serum below 4.76×10⁻⁴ is indicative of the disease presence with likelihood of 24.8%, compared to pre-test probability, and the PLR is 3.00.

TABLE 7
H1.0K180me2 antigen concentration normalized by total protein

| | | 95% |
|---|---|---|
| Metric | Value | confidence interval |
| Threshold | 4.76 × 10⁻⁴ | |
| Sensitivity | 90% | [56%, 100%] |
| Specificity | 70% | [35%, 93%] |
| PPV | 75% | [43%, 93%] |
| NPV | 88% | [47%, 99%] |
| LR+ | 3.00 | [1.14, 7.91] |
| LR− | 0.14 | [0.02, 0.93] |
| ln(OR) | 3.04 | [0.57, 5.51] |

Although serum concentrations of H1.0K180me2 are sufficient for identification of Alzheimer's disease patients, the use of serum sample normalization by total IgG (FIG. 12B) or total protein (FIG. 12C) allows for direct comparisons between individuals regardless of variables which may alter overall serum concentration, such as protocol used to obtain serum, operator variability, hydration state of patient and activity state of patient. IgGs are highly abundant in human serum and represent 11% of serum protein. As such, IgGs may act as indicators of serum concentration and may be used to normalize between patients. Total serum protein gives a direct indication of serum concentration and may be used to normalize concentrations of H1.0K180me2 between patients. In each instance, normalized H1.0K180me2 levels were observed to increase upon healthy aging (>60 years). Alzheimer's disease serum H1.0K180me2 levels were significantly lower than in age-matched controls, indicating that common normalization procedures do not alter the observed trend, whilst allowing for direct comparison of H1.0K180me2 levels between patients despite differences in serum collection and processing procedures, or basal concentration levels of patient sera.

### Anti-H1.0K180Me2 IgG (H1.0K180Me2 IgG Autoantibody) Levels

FIG. 13A shows that human anti-H1.0K180me2 IgG may be detected in different human biofluids using the indirect ELISA method. Human plasma, urine, and saliva were each tested for H1.0K180me2 IgG autoantibodies using the indirect ELISA test and a labeled H1.0K180me2 peptide. A linear relationship between plasma concentration and O.D. measured at 450 nm was detected when plasma was diluted 500×, 1000×, and 2000× in loading buffer. Similarly, a linear relationship between urine and saliva concentration and O.D. measured at 450 nm was detected when these fluids were diluted 80× and 160× in loading buffer. This suggests that the anti-H1.0K180me2 IgG indirect ELISA test has a utility to detect anti-H1.0K180me2 IgG in a wide variety of human biofluids.

In a related experiment anti-H1.0K180me2 IgG levels in human serum were quantified by indirect ELISA analysis using biotinylated-H1.0K180me2 capture peptide (an H1.0K180me2 autoantibody-binding peptide) followed by a secondary antibody specific for IgG antibodies. Equal volumes of serum from healthy individuals of 30-40 years (n=7) or >60 years (n=9), and individuals with clinically diagnosed Alzheimer's disease of >60 years (n=10) were analyzed. (FIG. 13B)

FIG. 13B shows the quantification of autoanti-H1.0K180me2 IgG levels determined by indirect ELISA in Alzheimer's disease patients and age-matched controls. The concentration of anti-H1.0K180me2 IgG (autoantibody IgG levels determined by indirect ELISA) in each serum sample was calculated using a standard curve created with serial dilutions of the H1.0K180me2 specific antibody included in the ELISA experiment shown in FIG. 12E. Alzheimer's disease patients display higher concentrations of serum anti-H1.0K180me2 IgG than healthy age-matched controls. Anti-H1.0K180me2 IgG serum concentrations may effectively segregate patients with Alzheimer's disease from healthy individuals and may act as a diagnostic tool for Alzheimer's disease detection. As shown in Table 8, measurement of auto antiH1.0K180me2 antibodies in concentration equal to, or higher than, 8.23 ug/ml is indicative of the disease presence with likelihood of 30%, compared to pre-test probability, with a PLR of 5.4. FIG. 13B shows standard curve the quantification of H1.0K180me2 autoantibodies in human serum. Total serum protein was measured for each sample using the Bradford assay. Anti-H1.0K180me2 IgG concentrations were normalized by measured protein concentration in each sample. Anti-H1.0K180me2 IgG levels are decreased in healthy >60 year individuals relative to healthy younger individuals (30-40 years). Patients with Alzheimer's disease exhibit increased normalized levels of anti-H1.0K180me2 IgG relative to healthy aged individuals (>60 years).

TABLE 8
Anti-H1.0K180me2 IgG concentration
normalized by serum volume

| | | 95% |
|---|---|---|
| Metric | Value | confidence interval |
| Threshold | 8.23 | |
| Sensitivity | 80% | [33%, 100%] |
| Specificity | 67% | [50%, 100%] |
| PPV | 73% | [63%, 100%] |
| NPV | 75% | [62%, 100%] |
| LR+ | 2.40 | [1.50, 15.45] |
| LR− | 0.30 | [0.00, 0.56] |
| ln(OR) | 2.08 | [0.00, 4.16] |

### Anti-H1.0K180Me2 IgM (H1.0K180Me2 IgM Autoantibody) Levels

In another related experiment, anti-H1.0K180me2 IgM levels in human serum were quantified by indirect ELISA analysis using biotinylated-H1.0K180me2 capture peptide (an H1.0K180me2 autoantibody-binding peptide), followed by a secondary antibody specific for IgM antibodies (normalized by volume). Equal volumes of serum from healthy individuals >60 years (n=9), and individuals with clinically diagnosed Alzheimer's disease of >60 years (n=10) were analyzed. (raw data in FIG. 14E; data normalized to total IgM levels in FIG. 15A).

Table 9 below shows the raw data from the indirect ELISA analysis, and further analyzed in FIGS. 14D-14E. The ELISA was performed in triplicate for each patient sample. The anti-H1.0K180me2 IgM concentration was calculated for each replicate from the standard curve shown in FIG. 14D. The average anti-H1.0K180me2 IgM concentration for each sample was calculated from the three technical replicates. The sample standard deviation between the three technical replicates was also calculated. The coefficient of embodiment between the replicates was calculated as CV %=standard deviation/average×100%.

As an anti-H1.0K180me2 standard curve was not available, the standard curve was created using an anti-H1.0K180me2 IgG antibody (FIG. 14D). The molar concentration of anti-H1.0K180me2 IgG was plotted against OD at 450 nm. The curve was then used to infer the molar concentration of anti-H1.0K180me2 IgM in each sample.

TABLE 9
Raw data from anti-H1.0K180me2 IgM ELISA

| | | | Repeat 1 | | Repeat 2 | | Repeat 3 | | | | |
|---|---|---|---|---|---|---|---|---|---|---|---|
| | | | | Anti-H1.0 | | Anti-H1.0 | | Anti-H1.0 | Average Anti- | | |
| Patient | | Patient | | K180me2 IgM | | K180me2 IgM | | K180me2 IgM | H1.0 K180me2 | Standard | |
| Group | Patient ID | Age | O.D. | (fmol/ml) | O.D. | (fmol/ml) | O.D. | (fmol/ml) | IgM (fmol/ml) | deviation | % CV |
| AD | NC17 | 75 | 0.18 | 498.9 | 0.18 | 510.1 | 0.16 | 431.5 | 480.1 | 42.5 | 8.9 |
| AD | NC14 | 77 | 0.46 | 1288.2 | 0.47 | 1321.9 | 0.47 | 1319.1 | 1309.7 | 18.7 | 1.4 |
| AD | NC15 | 78 | 0.23 | 653.4 | 0.20 | 566.3 | 0.18 | 498.9 | 572.8 | 77.5 | 13.5 |
| AD | NC11 | 83 | 0.13 | 358.4 | 0.10 | 285.4 | 0.11 | 313.5 | 319.1 | 36.8 | 11.5 |
| AD | NC20 | 90 | 0.38 | 1063.5 | 0.37 | 1021.3 | 0.38 | 1049.4 | 1044.8 | 21.5 | 2.1 |
| AD | NC18 | 91 | 0.49 | 1369.7 | 0.48 | 1350.0 | 0.36 | 1010.1 | 1243.3 | 202.1 | 16.3 |
| AD | NC19 | 93 | 0.16 | 434.3 | 0.15 | 428.7 | 0.16 | 456.7 | 439.9 | 14.9 | 3.4 |
| AD | NC13 | 94 | 0.19 | 518.5 | 0.18 | 512.9 | 0.19 | 515.7 | 515.7 | 2.8 | 0.5 |
| AD | NC12 | 103 | 0.06 | 156.2 | 0.05 | 147.8 | 0.06 | 173.0 | 159.0 | 12.9 | 8.1 |
| Control | WD-32339 | 63 | 0.06 | 175.8 | 0.05 | 139.3 | 0.06 | 153.4 | 156.2 | 18.4 | 11.8 |
| Control | WD-32347 | 66 | 0.06 | 164.6 | 0.05 | 142.1 | 0.05 | 147.8 | 151.5 | 11.7 | 7.7 |
| Control | WD-32398 | 70 | 0.20 | 557.9 | 0.20 | 569.1 | 0.20 | 566.3 | 564.4 | 5.8 | 1.0 |
| Control | WD-36002 | 70 | 0.13 | 347.2 | 0.12 | 344.4 | 0.16 | 456.7 | 382.8 | 64.1 | 16.7 |
| Control | WD-32351 | 70 | 0.12 | 344.4 | 0.11 | 313.5 | 0.12 | 330.3 | 329.4 | 15.5 | 4.7 |
| Control | WD-36008 | 73 | 0.07 | 184.3 | 0.07 | 192.7 | 0.08 | 215.2 | 197.4 | 16.0 | 8.1 |
| Control | WD-36010 | 74 | 0.06 | 161.8 | 0.07 | 184.3 | 0.05 | 142.1 | 162.7 | 21.1 | 13.0 |
| Control | WD-32346 | 76 | 0.15 | 420.2 | 0.14 | 389.3 | 0.15 | 417.4 | 409.0 | 17.1 | 4.2 |
| Control | WD-32350 | 66 | 0.15 | 417.4 | 0.16 | 451.1 | 0.20 | 543.8 | 470.8 | 65.5 | 13.9 |

FIG. 14E demonstrates the utility of measurements of IgG autoantibodies to H1.0K180me2 as biomarkers of Alzheimer's disease (figures show raw data). The left panel shows the ROC curve analysis, used to evaluate the overall predictive performance, and to pick an optimal threshold cutoff value, to distinguish between positive and negative test results. The relationship between percent specificity and sensitivity at each possible threshold are plotted as shown to create the empirical ROC curve (solid line). The empirical ROC curve was used to calculate the optimal threshold cutoff value pictured in the right panel. The optimal threshold value is shown as a gray dot on the empirical ROC curve. The right panel of FIG. 14E shows a box plot distribution of anti-H1.0K180me2 IgM concentrations in patients with Alzheimer's disease and those without (neurologic controls). Individual measurement values for all samples are shown as dots, and box-plots are shown for each distribution along with the upper quartile, lower quartile, and distance of the upper and lower quartile to the largest and lowest non-outlying points. Samples above the threshold cutoff (dashed line) are considered positive for the test. Samples below the threshold cutoff are considered negative for the test. The 2×2 matrix at the bottom of FIG. 14E shows the distribution may be divided into four groups: true positives (TP; where the index was positive, and AD was present); false positives (FP; where the index was positive, but AD was not present); true negatives (TN; where the index was negative, and AD was not present); and false negatives (FN; where the index was negative, but AD was present). The numbers of samples that fall within each group were plotted on the 2×2 matrix.

Table 10 below shows the assessment of anti-H1.0K180me2 IgM test performance, for the raw data.

TABLE 10
Assessment of anti-H1.0K180me2 test performance

| | | 95% |
|---|---|---|
| Metric | Value | confidence interval |
| Threshold | 409 | |
| Sensitivity | 78% | [45%, 94%] |
| Specificity | 78% | [45%, 94%] |
| PPV | 78% | [45%, 94%] |
| NPV | 78% | [45%, 94%] |
| LR+ | 3.50 | [0.98, 12.47] |
| LR− | 0.29 | [0.08, 1.02] |
| ln(OR) | 2.51 | [0.28, 4.73] |

Table 11 below shows the normalized data (anti-H1.0K180me2 IgM normalized by total IgM levels) from the indirect ELISA analysis, and further analyzed in FIG. 15A, FIG. 15B. The ELISA was performed in triplicate for each patient sample. The anti-H1.0K180me2 IgM concentration was calculated for each replicate from the standard curve shown in FIG. 14D, and normalized by the average total IgM concentration measured in the sample (FIG. 14B). The average, normalized anti-H1.0K180me2 IgM concentration for each sample was calculated from the three technical replicates. The sample standard deviation between the three technical replicates was also calculated. The coefficient of embodiment between the replicates was calculated as CV %=standard deviation/average×100%.

TABLE 11
Normalized data (by total IgM levels) from anti-H1.0K180me2 IgM ELISA

| | | | Ratio of anti-H1.0K180me2 IgM | | | | | |
|---|---|---|---|---|---|---|---|---|
| Patient | | Patient | to total IgM (×10⁻⁶) | | | | Standard | |
| Group | Patient ID | Age | Repeat 1 | Repeat 2 | Repeat 3 | Average | deviation | % CV |
| AD | NC17 | 75 | 87.0 | 89.0 | 75.3 | 83.7 | 7.4 | 8.9 |
| AD | NC14 | 77 | 80.2 | 82.3 | 82.1 | 81.5 | 1.2 | 1.4 |
| AD | NC15 | 78 | 53.6 | 46.4 | 40.9 | 47.0 | 6.3 | 13.5 |
| AD | NC11 | 83 | 23.3 | 18.5 | 20.4 | 20.7 | 2.4 | 11.5 |
| AD | NC20 | 90 | 32.5 | 31.2 | 32.1 | 31.9 | 0.7 | 2.1 |
| AD | NC18 | 91 | 41.3 | 40.7 | 30.4 | 37.5 | 6.1 | 16.3 |
| AD | NC19 | 93 | 27.4 | 27.0 | 28.8 | 27.7 | 0.9 | 3.4 |
| AD | NC13 | 94 | 74.3 | 73.5 | 73.9 | 73.9 | 0.4 | 0.5 |
| AD | NC12 | 103 | 4.3 | 4.0 | 4.7 | 4.3 | 0.4 | 8.1 |
| Control | WD-32339 | 63 | 18.6 | 14.7 | 16.2 | 16.5 | 1.9 | 11.8 |
| Control | WD-32347 | 66 | 9.6 | 8.3 | 8.6 | 8.8 | 0.7 | 7.7 |
| Control | WD-32350 | 66 | 22.2 | 23.9 | 28.9 | 25.0 | 3.5 | 13.9 |
| Control | WD-32398 | 70 | 23.9 | 24.4 | 24.3 | 24.2 | 0.3 | 1.0 |
| Control | WD-36002 | 70 | 22.9 | 22.7 | 30.1 | 25.3 | 4.2 | 16.7 |
| Control | WD-32351 | 70 | 18.8 | 17.1 | 18.0 | 18.0 | 0.8 | 4.7 |
| Control | WD-36008 | 73 | 6.3 | 6.6 | 7.3 | 6.7 | 0.5 | 8.1 |
| Control | WD-36010 | 74 | 10.0 | 11.4 | 8.8 | 10.1 | 1.3 | 13.0 |
| Control | WD-32346 | 76 | 27.3 | 25.3 | 27.1 | 26.6 | 1.1 | 4.2 |

FIG. 15A demonstrates the utility of measurements of IgM autoantibodies to H1.0K180me2 as biomarkers of Alzheimer's disease (figures show normalized data). The left panel shows the ROC curve analysis, used to evaluate the overall predictive performance, and to pick an optimal threshold cutoff value, to distinguish between positive and negative test results. The relationship between percent specificity and sensitivity at each possible threshold are plotted as shown to create the empirical ROC curve (solid line). The empirical ROC curve was used to calculate the optimal threshold cutoff value pictured in the right panel. The optimal threshold value is shown as a gray dot on the empirical ROC curve. The right panel of FIG. 15A shows a box plot distribution of normalized anti-H1.0K180me2 IgM concentrations in patients with Alzheimer's disease and those without (neurologic controls). Individual measurement values for all samples are shown as dots, and box-plots are shown for each distribution. The box plots show median values along with the upper quartile, lower quartile, and distance of the upper and lower quartile to the largest and lowest non-outlying points. Samples above the threshold cutoff (dashed line) are considered positive for the test. Samples below the threshold cutoff are considered negative for the test. The 2×2 matrix at the bottom of FIG. 15A shows the distribution may be divided into four groups: true positives (TP; where the index was positive, and AD was present); false positives (FP; where the index was positive, but AD was not present); true negatives (TN; where the index was negative, and AD was not present); and false negatives (FN; where the index was negative, but AD was present). The numbers of samples that fall within each group were plotted on the 2×2 matrix. FIG. 15B demonstrates that test characteristics do not fluctuate depending on laboratory setting and different operators.

Table 12 below shows the assessment of anti-H1.0K180me2 IgM/total IgM test performance.

TABLE 12

| | | 95% |
|---|---|---|
| Metric | Value | confidence interval |
| Threshold | 26.6 | |
| Sensitivity | 75% | [44%, 92%] |
| Specificity | 95% | [66%, 99%] |
| PPV | 94% | [53%, 100%] |
| NPV | 79% | [47%, 95%] |
| LR+ | 15.00 | [0.98, 228.9] |
| LR− | 0.26 | [0.09, 0.78] |
| ln(OR) | 4.04 | [0.86, 7.23] |

The results of this test modify the probability of disease from 10% pre-test to 63% post-test. The positive likelihood ratio (LR+) is 15.0 (95% CI: 0.98, 229), and the positive predictive value (PPV) is 94% (95% CI: 53, 100). The negative likelihood ratio (LR−) is 0.26 (95% CI: 0.09, 0.78), and negative predictive value is 79% (95% CI: 47, 95).

### Controls

FIG. 14B shows a standard curve: the molar concentration of a human IgG dilution series was plotted against OD at 450 nm. The curve was then used to extrapolate the molar concertation of total IgM in each sample.

Table 13 below shows the data from a total IgM ELISA. The ELISA was performed in triplicate for each patient sample. The total IgM molar concentration was calculated for each replicate from the standard curve shown in FIG. 14B. The average total IgM molar concentration for each sample was calculated from the three technical replicates. The sample standard deviation between the three technical replicates was also calculated. The coefficient of embodiment between the replicates was calculated as CV %=standard deviation/average×100%.

TABLE 13
Measurement of total IgM levels in patient serum using an ELISA assay

| | | | Repeat 1 | | Repeat 2 | | Repeat 3 | | | | |
|---|---|---|---|---|---|---|---|---|---|---|---|
| Patient | | Patient | | Total IgM | | Total IgM | | Total IgM | Average Total | Standard | |
| Group | Patient ID | Age | O.D. | (nmol/ml) | O.D. | (nmol/ml) | O.D. | (nmol/ml) | IgM (nmol/ml) | deviation | % CV |
| AD | NC17 | 75 | 0.11 | 5.9 | 0.11 | 5.3 | 0.11 | 5.9 | 5.7 | 0.3 | 6.0 |
| AD | NC14 | 77 | 0.22 | 13.2 | 0.31 | 18.7 | 0.27 | 16.3 | 16.1 | 2.7 | 17.1 |
| AD | NC15 | 78 | 0.21 | 12.5 | 0.22 | 12.8 | 0.20 | 11.3 | 12.2 | 0.8 | 6.3 |
| AD | NC11 | 83 | 0.26 | 15.7 | 0.25 | 15.3 | 0.25 | 15.2 | 15.4 | 0.3 | 1.9 |
| AD | NC20 | 90 | 0.51 | 32.6 | 0.52 | 32.7 | 0.52 | 32.8 | 32.7 | 0.1 | 0.3 |
| AD | NC18 | 91 | 0.51 | 32.1 | 0.52 | 33.1 | 0.54 | 34.3 | 33.2 | 1.1 | 3.2 |
| AD | NC19 | 93 | 0.27 | 16.4 | 0.26 | 15.7 | 0.26 | 15.5 | 15.9 | 0.5 | 2.9 |
| AD | NC13 | 94 | 0.12 | 6.4 | 0.15 | 8.1 | 0.12 | 6.5 | 7.0 | 0.9 | 13.5 |
| AD | NC12 | 103 | 0.65 | 41.4 | 0.53 | 33.7 | 0.55 | 35.1 | 36.7 | 4.1 | 11.2 |
| Control | WD-32339 | 63 | 0.17 | 9.8 | 0.17 | 9.4 | 0.16 | 9.2 | 9.5 | 0.3 | 3.2 |
| Control | WD-32347 | 66 | 0.27 | 16.6 | 0.29 | 17.3 | 0.29 | 17.5 | 17.2 | 0.5 | 2.9 |
| Control | WD-32398 | 70 | 0.38 | 23.3 | 0.37 | 23.1 | 0.38 | 23.6 | 23.3 | 0.3 | 1.1 |
| Control | WD-36002 | 70 | 0.25 | 15.1 | 0.25 | 15.2 | 0.25 | 15.1 | 15.2 | 0.0 | 0.3 |
| Control | WD-32351 | 70 | 0.29 | 17.9 | 0.31 | 18.8 | 0.30 | 18.2 | 18.3 | 0.4 | 2.4 |
| Control | WD-36008 | 73 | 0.46 | 29.1 | 0.48 | 30.4 | 0.46 | 28.7 | 29.4 | 0.9 | 3.1 |
| Control | WD-36010 | 74 | 0.27 | 16.5 | 0.26 | 15.9 | 0.27 | 16.1 | 16.2 | 0.3 | 1.9 |
| Control | WD-32346 | 76 | 0.25 | 14.7 | 0.27 | 16.1 | 0.26 | 15.4 | 15.4 | 0.7 | 4.8 |
| Control | WD-32350 | 66 | 0.29 | 17.4 | 0.31 | 18.8 | 0.33 | 20.3 | 18.8 | 1.5 | 7.8 |

FIG. 14B demonstrates that total IgM levels do not discriminate between individuals with and without Alzheimer's disease. Individual measurement values for all samples are shown as dots, and box-plots are shown for each distribution. The box-plots show the median values along with the upper quartile, lower quartile, and distance of the upper and lower quartile to the largest and lowest non-outlying points.

FIG. 16A demonstrates that there is no correlation between total IgM levels and anti-H1.0K180me2 IgM levels in patient samples. All patient samples were distributed on a scatter plot based on their measured anti-H1.0K180me2 IgM concentrations versus their measured total IgM concentration. Anti-H1.0K180me2 IgM levels are not influenced by total IgM levels in patient serum, as evidenced by the low R² value.

FIG. 16B demonstrates that total unmodified H1.0 protein levels (left graph) and total anti-H1.0 IgM levels do not discriminate between individuals with and without Alzheimer's disease. In the left panel, a custom chemiluminescent slot-blot immunoassay was used to measure total H1.0 (unmodified) levels in Alzheimer's disease and control patient serum samples. In the right panel, an indirect ELISA was used to measure IgM autoantibodies against H1.0 unmodified peptide in patient serum. There was no statistically significant difference in the levels of IgM autoantibodies against unmodified H1.0 peptide (p=0.72), indicating diagnostic utility for Alzheimer's is unique to anti-H1.0K180me2 IgM in patient serum. Relative measurement values for both panels, for all samples, are shown as dots, and box-plots are shown for each distribution. The box-plots show the median values along with the upper quartile, lower quartile, and distance of the upper and lower quartile to the largest and lowest non-outlying points.

### Correlation of Anti-H1.0K180me2 IgG and IgM Levels

FIG. 17 demonstrates that measuring H1.0K180me2 IgG and IgM autoantibodies may stratify patients with Alzheimer's disease into distinct populations. Anti-H1.0K180me2 IgG levels were measured in Alzheimer's disease patient samples, and these were normalized by total IgG levels. Normalized anti-H1.0K180me2 IgM levels in Alzheimer's disease patient samples were then correlated with these normalized anti-H1.0K180me2 IgG levels via scatter plot. This analysis allows for stratification of the Alzheimer's disease patients into distinct populations. Patients marked by 75, 77, 94, and 78 in FIG. 17 will likely respond to any Alzheimer's disease treatment. Patients marked by 90, 91, 93, 83, and 103 in FIG. 17 will likely respond to only specific Alzheimer's disease treatments, for example only non-immunomodulatory Alzheimer's disease treatments.

### Example 10: Rapamycin and its Derivatives Block the Accumulation of H1.0K180Me2 in the Cytoplasm, Following DNA Damage

To test if rapamycin derivatives may block H1.0K180me2 appearance upon DNA damage, SR hADSCs were treated with bleomycin for 2 hours with or without pretreatment with rapamycin or everolimus (a derivative of rapamycin) for 24 hours. Cells were then lysed and analyzed be western blot for H1.0K180me2, γH2A.X and β-Actin, according to methods described in Example 1. As shown in FIG. 18, both rapamycin and everolimus reduced the appearance of H1.0K180me2 upon bleomycin treatment, suggesting everolimus may also block H1.0K180me2 appearance upon DNA damage.

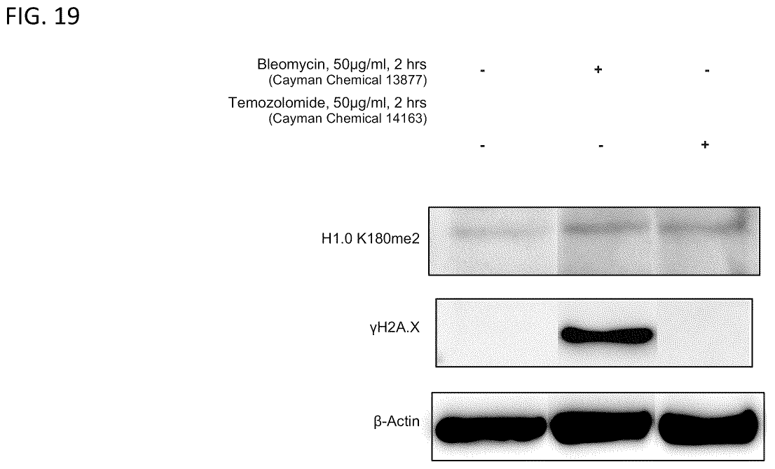

SR hADSCs were treated for 2 hours with bleomycin or temozolomide, a compound that triggers base excision repair pathways. Cells were then lysed and analyzed be western blot for H1.0K180me2, γH2A.X and β-Actin. As shown in FIG. 19, both bleomycin and temozolomide were capable of inducing H1.0K180me2 methylation.

### Example 11: mTOR and PI3K Inhibitors Block the Accumulation of H1.0K180Me2 Following DNA Damage

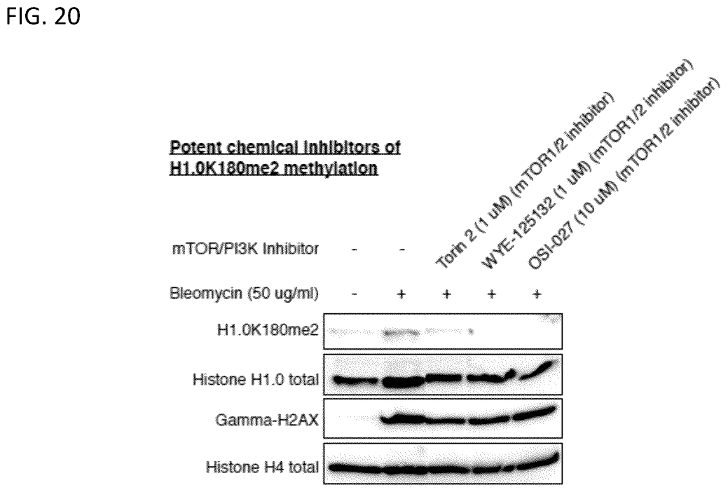

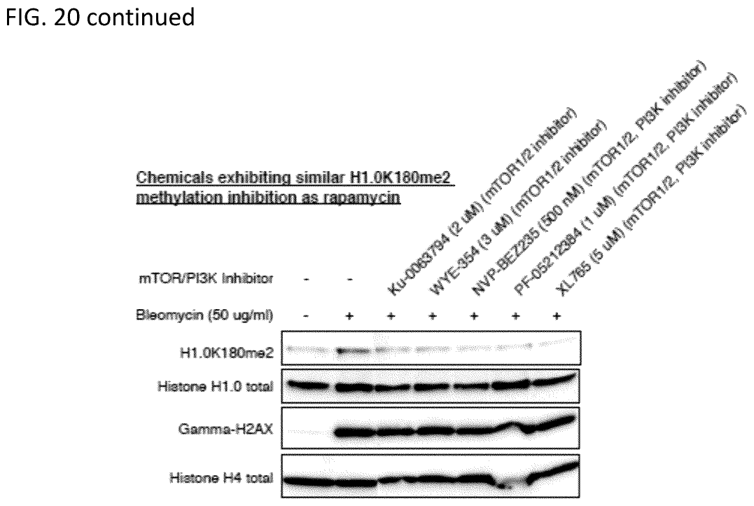

FIG. 20 shows the effect of effect of mTOR and PI3K inhibitors on H1.0K180me2 dynamics. SR hADSCs were treated with bleomycin for 2 hours with or without 24 hours pretreatment with chemical inhibitors of mTOR1, mTOR2 and/or PI3K. Cells were lysed and chromatin extracted for analysis by western blot for H1.0K180me2, H1.0 total, γH2A.X and histone H4 total. Drug concentration used, and the specific inhibitory targets for each drug are given next to drug names. All inhibitors tested were capable of reducing the appearance of H1.0K180me2 upon bleomycin treatment.

### Example 12: In Vitro Methylation with G9A and Analysis of Products

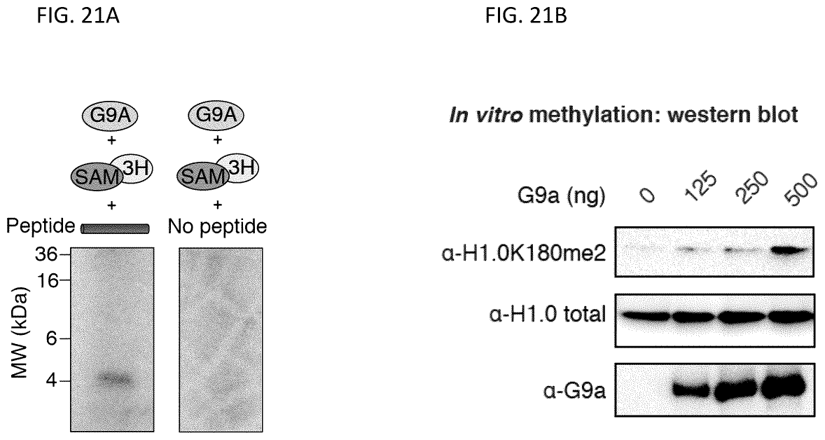

FIGS. 21A-21B show the results of this in vitro G9A methylation assay.

The G9A methyltransferase capable of methylating an H1.0 peptide (FIG. 21A). The methylation reaction was subsequently resolved on a gel and visualized via autoradiography. The appearance of a band at 4 kDa shows that The G9A methyltransferase capable of methylating H1.0 peptide through the transfer of tritium-labeled methyl groups to the peptide, allowing for visualization. A control reaction lacking H1.0 peptide yielded no tritium-labeled products.

The G9A methyltransferase capable of methylating a full-length recombination H1.0 (FIG. 21B). An in vitro methylation assay of recombinant full-length histone H1.0 with increasing amounts of G9A is shown in FIG. 21B. G9A is capable of dimethylating full length H1.0 at K180.

In order to identify the precise locations of G9A methylation on unmodified H1.0 peptide (AKPVKASKPKKAKPVKPK (SEQ ID NO:42)), a methylation reaction was set up and the products identified by LC-MS. The methylation reaction comprised recombinant G9A, an unlabeled methyl donor (S-Adenosyl-L-Methionine), and unmodified H1.0 peptide. The methylation reaction was subsequently analyzed by LC-MS, and each spectral peak (corresponding to a peptide species in the final reaction) was identified and quantified using spectral counts. The number of “me” circles in FIG. 22A represents the methylation state of the lysine residue (mono-, di-, or tri-methylated). FIG. 22A shows that in the presence of unmethylated H1.0 peptide, G9A specifically and abundantly dimethylates H1.0K180 (99.9% of all peptides).

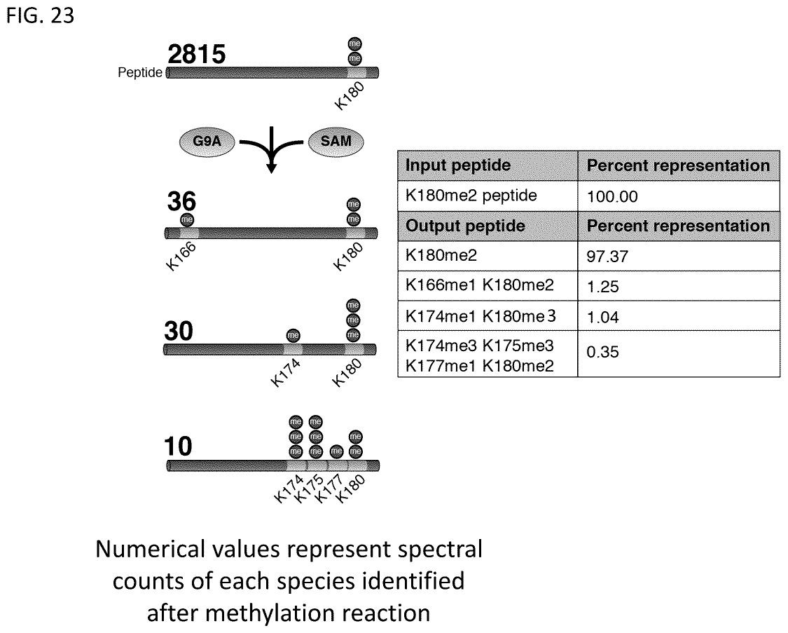

More specifically, the data demonstrate that G9A dimethylates lysine K180 but not other lysines (K166, K174, K175 or K177) present in the same peptide fragment, with an estimated methylation efficiency of about 99% (FIG. 22A and FIG. 22B). To further address the sensitivity and specificity of G9A methylation to lysine 180 of H1.0 peptide, the K180me2 peptide (H1.0 AA 165-182) was used as a substrate in similar in vitro methylation experiments. Only minor quantitates of further methylated peptide were detected: H1.0K166me1K180me2 (1.27% of the total peptide in the reaction), H1.0K174me1K180me3 (1.06% of the total peptide in the reaction) and H1.0K174me3K175me3K177me1K180me2 (0.35% of the total peptide in the reaction) as shown in FIG. 22B, FIG. 23.

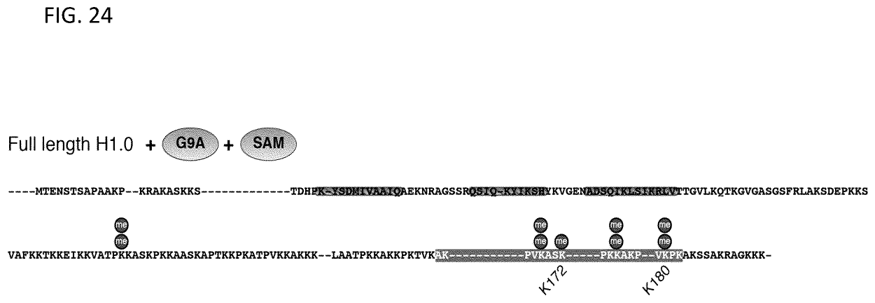

In order to identify the precise locations of G9A methylation on full-length H1.0 protein, a methylation reaction was set up and the products identified by LC-MS. The methylation reaction comprised recombinant G9A, an unlabeled methyl donor (S-Adenosyl-L-Methionine), and recombinant human H1.0 protein. The methylation reaction was subsequently analyzed by LC-MS, and sites of methylation were identified. The number of “me” circles in FIG. 24 represents the methylation state of the lysine residue (mono-, di-, or tri-methylated). FIG. 24 shows that in the presence of recombinant, full-length H1.0, G9A methylates C-terminal lysine residues, including H1.0K180me2.

### Example 13: In Vitro Methylation with GLP and Analysis of Products

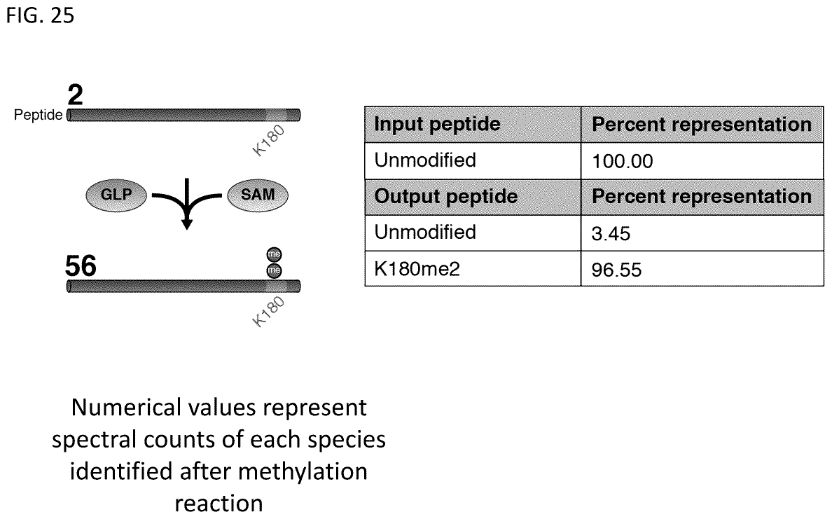

In order to identify the precise locations of GLP methylation on unmodified H1.0 peptide (AKPVKASKPKKAKPVKPK (SEQ ID NO:42)), a methylation reaction was set up and the products identified by LC-MS. The methylation reaction comprised recombinant GLP, an unlabeled methyl donor (S-Adenosyl-L-Methionine), and unmodified H1.0 peptide. The methylation reaction was subsequently analyzed by LC-MS, and each spectral peak (corresponding to a peptide species in the final reaction) was identified and quantified using spectral counts. The number of “me” circles in FIG. 25 represents the methylation state of the lysine residue (mono-, di-, or tri-methylated). FIG. 25 shows that in the presence of unmethylated H1.0 peptide, GLP specifically dimethylates H1.0K180 (96.6% of all peptides).

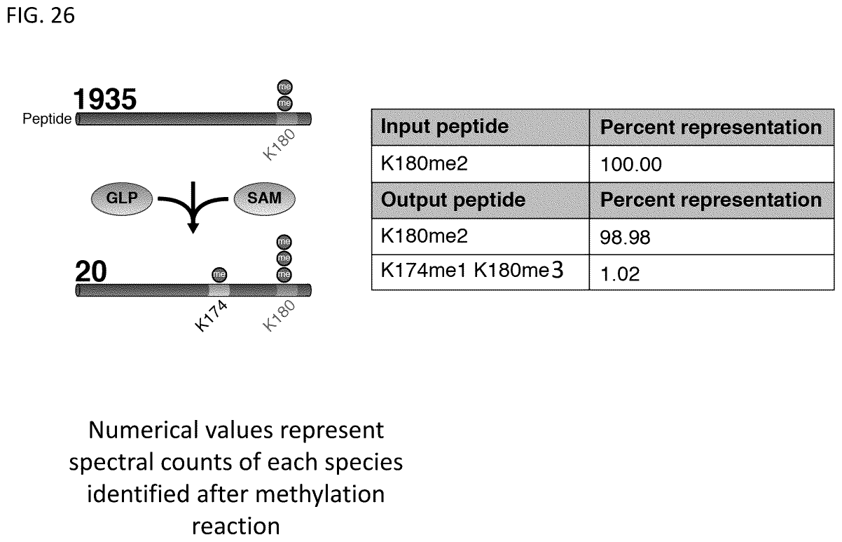

In order to identify the precise locations of GLP methylation on K180 dimethylated H1.0 peptide (AKPVKASKPKKAKPVK^{(me2)}PK (SEQ ID NO:3)), a methylation reaction was set up and the products identified by LC-MS. The methylation reaction comprised recombinant GLP, an unlabeled methyl donor (S-Adenosyl-L-Methionine), and H1.0K180me2 peptide. The methylation reaction was subsequently analyzed by LC-MS, and each spectral peak (corresponding to a peptide species in the final reaction) was identified and quantified using spectral counts. The number of “me” circles in FIG. 26 represents the methylation state of the lysine residue (mono-, di-, or tri-methylated). FIG. 26 shows that in the presence of K180 dimethylated H1.0 peptide, GLP only further methylates H1.0K180 and H1.0K174, and only with a very low efficiency (1.02% of peptides are further methylated).

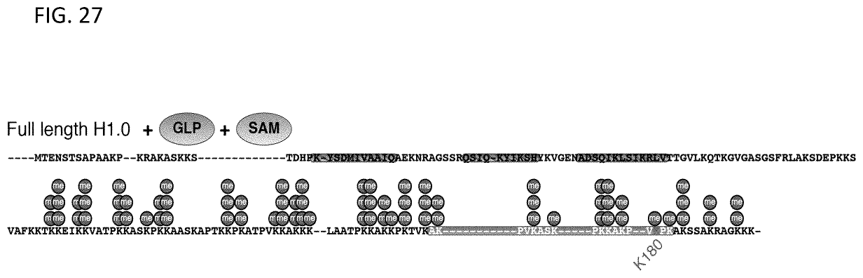

In order to identify the precise locations of GLP methylation on full-length H1.0 protein, a methylation reaction was set up and the products identified by LC-MS. The methylation reaction comprised recombinant GLP, an unlabeled methyl donor (S-Adenosyl-L-Methionine), and recombinant human H1.0 protein. The methylation reaction was subsequently analyzed by LC-MS, and sites of methylation were identified. The number of “me” circles in FIG. 27 represents the methylation state of the lysine residue (mono-, di-, or tri-methylated). FIG. 27 shows that in the presence of recombinant full-length H1.0, under the conditions described herein, GLP does not methylated full length H1.0. GLP methylates a large number of lysine residues on the C-terminal tail of H1.0, lacking the specificity of G9A, under the conditions provided herein.

### Example 14: siRNA Knockdown of G9A in hADSCs

siRNA Transfection

siRNA pools designed to target G9A were obtained from Qiagen (GS10919) and random scrambled siRNA pools were obtained from ThermoFisher Scientific (4390843). siRNA pools were transfected into self-replicating hADSCs using Lipofectamine 3000 (Life Technologies) following manufacturer's protocols. Cells were collected 24 hours after siRNA transfection and analyzed by qPCR or western blot.

qPCR

Cell cultures were homogenized in Trizol (Invitrogen) and total RNA was isolated using the RNeasy kit (Qiagen). The RNA was quantified with Qubit (Invitrogen) and reverse transcribed using SuperScript III following manufacturer's protocol (Invitrogen). Quantitative PCR analyses were performed in triplicate using an Applied Biosystems 7700 sequence detector with ˜5 ng of cDNA, 1 μM designated primer pairs and Fast-SYBR Green PCR master mix following manufacturers protocol (Applied Biosystems). Primer pairs are listed below. The mean cycle threshold (Ct) for each gene was normalized to levels of beta-actin in the same sample (delta Ct). Unpaired two-sample t-tests were used to determine differences in mean delta Ct values between treatment groups. Where appropriate, the fold change was calculated by the delta-delta Ct method (fold=2ΔΔCt).

TABLE 14
Primers

| | |
|---|---|
| GLP-Forward | 5′-AGGGGAGTGCTGACACAGAG-3′ |
| | (SEQ ID NO: 107) |
| GLP-Reverse | 5′-GGGATCTTTACTGGCTGCAT-3′ |
| | (SEQ ID NO: 80) |
| Beta-actin-Forward | 5′-CTCTTCCAGCCTTCCTTCCT-3′ |
| | (SEQ ID NO: 81) |
| Beta-actin-Reverse | 5′-AGCACTGTGTTGGCGTACAG-3′ |
| | (SEQ ID NO: 82) |
| H1.0-Forward | 5′-CTCAAGCAGACCAAAGGGGT-3′ |
| | (SEQ ID NO: 83) |
| H1.0-Reverse | 5′-GGCGTGGCTACCTTCTTGAT-3′ |
| | (SEQ ID NO: 84) |
| H1.1-Forward | 5′-AGGCAACGGGTGCATCTAAA-3′ |
| | (SEQ ID NO: 85) |
| H1.1-Reverse | 5′-GATTTCCTTGTTGCCGCAGG-3′ |
| | (SEQ ID NO: 86) |
| H1.2-Forward | 5′-CAAAGAAGGCCAAGGTTGCG-3′ |
| | (SEQ ID NO: 87) |
| H1.2-Reverse | 5′-CGCCTTCTTAGGCTTGACAAC-3′ |
| | (SEQ ID NO: 88) |
| H1.3-Forward | 5′-AGTGGCCAAGAGTGCGAAAA-3′ |
| | (SEQ ID NO: 89) |
| H1.3-Reverse | 5′-CTTCGGCTTCCCCGACTTAG-3′ |
| | (SEQ ID NO: 90) |
| H1.4-Forward | 5′-ACGCTTGCCTTCAACATGTCC-3′ |
| | (SEQ ID NO: 91) |
| H1.4-Reverse | 5′-AGTAATGAGCTCGGACACCG-3′ |
| | (SEQ ID NO: 92) |
| H1.5-Forward | 5′-CCGGCTAAGAAGAAGGCAAC-3′ |
| | (SEQ ID NO: 93) |
| H1.5-Reverse | 5′-GCTCCTTAGAAGCAGCCACA-3′ |
| | (SEQ ID NO: 94) |
| G9A-Forward | 5′-TGCTGAGGCTGATGTGAGAG-3′ |
| | (SEQ ID NO: 95) |
| G9A-Reverse | 5′-GGTCACACAGGTGGTTGATG-3′ |
| | (SEQ ID NO: 96) |

### Results

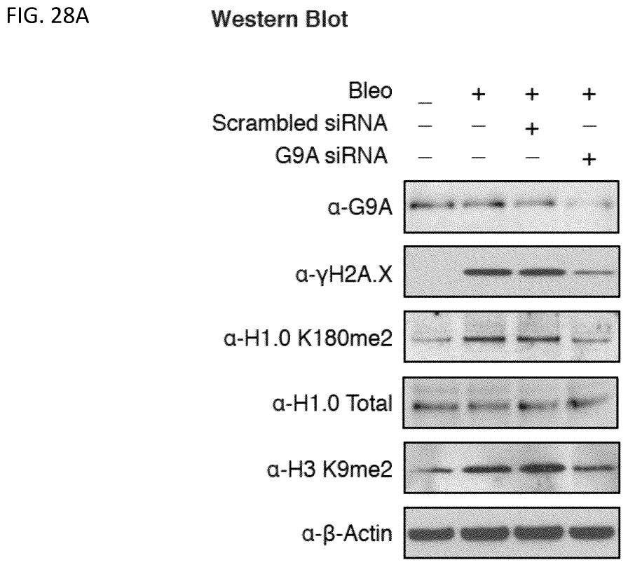

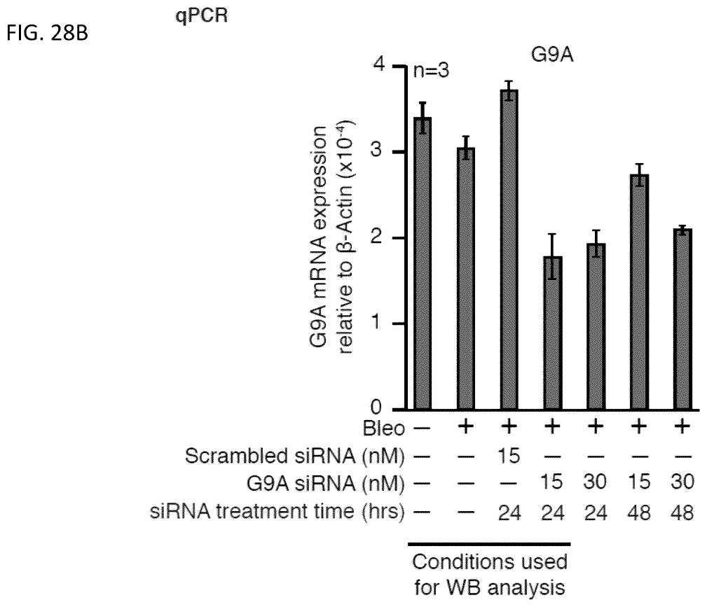

Self renewing (SR) human adipose derived stem cells (hADSCs) were transfected with either scrambled control siRNA or siRNA targeting G9A. The cells were then subjected to 2 hours bleomycin treatment. Western blot analysis indicated knockdown of G9A in vivo results in reduced H1.0K180me2 appearance upon DNA damage. H3K9me2, a known methylation product of G9A, was used to monitor loss of G9A activity upon knockdown. (FIG. 28A)

The siRNA knockdown of G9A in hADSCs led to a significant reduction in H1.0K180me2 levels upon 2 hours bleomycin treatment (ADD) concomitantly with a reduction of H3K9me2, a known PTM provided by G9A (FIG. 28B).

### Example 15: Production of In Vitro Methylated H1.0K180Me2 Proteins or Peptides in Bulk

As observed in FIG. 21B and FIG. 24, G9A is capable of efficiently and specifically dimethylating K180 on full length recombinant human H1.0. This process may be utilized in order to create large quantities of H1.0K180me2 full length protein that could be incorporated into ELISA kits (e.g. sandwich ELISA kits) as a reference standard for the detection of H1.0K180me2 in biological samples. This process could involve setting up a bulk methylation reaction comprising 1×HMT reaction buffer (50 mM Tris-HCl, 5 mM MgCl₂, 4 mM dithiothreitol, pH 9.0), recombinant human or mouse G9A Methyltransferase, 3.2 mM S-Adenosyl-L-Methionine, and recombinant full-length human H1.0 labeled with an N-terminal tag (HA, His, GST, FLAG or other tag suitable for downstream purification). The reaction could be incubated at 37° C. for 1 hour to enable G9A-mediated H1.0K180me2 methylation.

Full-length H1.0 containing K180me2 would then be enriched and purified in a one or two-step process. First, all full-length recombinant H1.0 species in the reaction would be purified away from the reaction mixture through affinity purification utilizing the N-terminal tag. In one instance, H1.0 could be labeled with HA tag and purified with an HA antibody immobilized on a column or resin. After purification, the tag could be removed through proteolytic cleavage if required. This process would enrich for only full-length H1.0K180me2 in the final product, ensuring both capture and detection epitopes were present. The second step would utilize an H1.0K180me2 antibody immobilized on a column or resin, in order to further purify only full length H1.0 species containing H1.0K180me2. This final product could then be concentrated (for example using lyophilization), quantified, and included in ELISA kits as a reference standard, or used as a therapeutic. A one step purification approach utilizing only H1.0K180me2 antibody purification may be sufficient in some instances.

1.-15. (canceled)

16. A synthetic histone H1.0 peptide comprising a dimethylated lysine residue, wherein the dimethylated lysine residue corresponds to K180 of a human histone H1.0.

17. The peptide of claim 16, wherein the peptide comprises a label.

18. The peptide of claim 16, wherein the peptide is biotinylated.

19. The peptide of claim 16, wherein the peptide does not comprise any other lysine residues that are dimethylated.

20.-21. (canceled)

22. The peptide of claim 16, wherein the peptide comprises any one of the sequences of SEQ ID NOS:3-35.

23.-25. (canceled)

26. The peptide of claim 16, wherein the peptide is capable of clearing or blocking H1.0K180me2 autoantibodies.

27.-182. (canceled)

183. The peptide of claim 16, wherein the peptide has a length of between 5 amino acids and 30 amino acids.

184. The peptide of claim 22, wherein the peptide consists of a sequence selected from the group consisting of SEQ ID NOS:3-35.

185. The peptide of claim 16, wherein the peptide is attached to a bead, microplate, column, or resin.

186. The peptide of claim 16, wherein the peptide is conjugated to at least one therapeutic agent selected from the group consisting of a radionuclide, a cytotoxin, a chemotherapeutic agent, a drug, a pro-drug, a toxin, an enzyme, an immunomodulator, a pro-apoptotic agent, a cytokine, a hormone, an oligonucleotide, an antisense molecule, a siRNA, and a second antibody.

187. The peptide of claim 26, wherein the H1.0K180me2 autoantibodies comprise IgG antibodies, IgG antibodies, or a combination thereof.

188. A diagnostic kit comprising the peptide of claim 1.

189. The kit of claim 188, wherein the peptide is provided as a reference standard.

190. The kit of claim 188, wherein the kit further comprises an anti-H1.0K180me2 antibody.

191. A transdermal patch for measuring a concentration of a hypodermal target molecule, comprising:
(a) a substrate comprising the peptide of claim 1; and
(b) a plurality of microneedles.

192. The patch of claim 191, wherein the patch is configured as a transdermal microneedle array patch.

193. The patch of claim 191, wherein the substrate is elastically stretchable.

194. A portable unit for determining whether an individual has been exposed to radiation or a DNA-damaging agent, comprising:
(a) a sample collection unit;
(b) a reader;
(c) an assay module comprising the peptide of claim 1; and
(d) a plurality of microneedles.

195. A test strip suitable for a lateral flow assay of an analyte, comprising a sample receiving zone, wherein the sample receiving zone comprises the peptide of claim 1.

196. A pharmaceutical composition comprising the peptide of claim 1 and a pharmaceutically acceptable excipient.
